SEQUENCE LISTING (66 sequences)

SEQ ID NO: 1 - protein, 15 aa, synthetic construct
KGHKIRGTVVLMRKN

SEQ ID NO: 2 - protein, 19 aa, synthetic construct
VDVLNLDGCTIDEALASGR

SEQ ID NO: 3 - DNA, 321 bp, Pisum sativum
  source 1..321: mol_type = genomic DNA
gatccgaatagcgagaaaccagctacagaaacatatgttccaagagatgaaaattttggtcacttgaagtcctctgactttcttgcatatggaataaaatctgtatctcaatgtgtagtacctgcatttgaatctgcatttgatttgaattttacgcctaacgagtttgatagttttcaagatgtgcgcaacctcttcgaaggtggaattaagcttcctctagatgtaattagcacaattagtcctttacctgtggtcaaagaaatctttcgtaccgatggtgaacaagtcctcaagtttacaccaccacatgtcattcga

SEQ ID NO: 4 - DNA, 24 bp, synthetic construct
  source 1..24: mol_type = other DNA
gtaccgatggtgaacaagtcctca

SEQ ID NO: 5 - DNA, 310 bp, synthetic construct
  source 1..310: mol_type = other DNA
gatccgaatagcgagaaaccagctacagaaacatatgttccaagagatgaaaattttggtcacttgaagtcctctgactttcttgcatatggaataaaatctgtatctcaatgtgtagtacctgcatttgaatctgcatttgatttgaattttacgcctaacgagtttgatagttttcaagatgtgcgcaacctcttcgaaggtggaattaagcttcctctagatgtaattagcacaattagtcctttacctgtggtcaaagaaatctttcgtaccgatggtgaagtttacaccaccacatgtcattcga

SEQ ID NO: 6 - DNA, 313 bp, synthetic construct
  source 1..313: mol_type = other DNA
gatccgaatagcgagaaaccagctacagaaacatatgttccaagagatgaaaattttggtcacttgaagtcctctgactttcttgcatatggaataaaatctgtatctcaatgtgtagtacctgcatttgaatctgcatttgatttgaattttacgcctaacgagtttgatagttttcaagatgtgcgcaacctcttcgaaggtggaattaagcttcctctagatgtaattagcacaattagtcctttacctgtggtcaaagaaatctttcgtaccgatggtgaacaagtttacaccaccacatgtcattcga

SEQ ID NO: 7 - protein, 863 aa, Pisum sativum
MFPNVTGLLNKGHKIRGTVVLMRKNVLDFNTIVSIGGGNVHGVIDSGINIIGSTLDGLTAFLGRSVSLQLISATKSDANGKGKVGKDTFLEGVLASLPTLGAGESAFNIHFEWDHEMGIPGAFYIKNYMQVEFFLKSLTLEDVPNHGTIRFVCNSWVYNSKLYKSPRIFFANKSYLPSETPSPLVKYREEELQTLRGDGTGERKLHERIYDYDVYNDLGNPDHGEHLARPILGGSSTHPYPRRGRTGRYPTRKDPNSEKPATETYVPRDENFGHLKSSDFLAYGIKSVSQCVVPAFESAFDLNFTPNEFDSFQDVRNLFEGGIKLPLDVISTISPLPVVKEIFRTDGEQVLKFTPPHVIRVSKSAWMTDEEFAREMLAGVNPCMIRGLQEFPPKSNLDPAEYGDHTSKISVDVLNLDGCTIDEALASGRLFILDYHDTFIPFLRRINETSAKAYATRTILFLKENGTLKPVAIELSLPHPDGDKSGFVSKVILPADEGVESTIWLLAKAYVVVNDSCYHQLMSHWLNTHAVIEPFVIATNRQLSVVHPINKLLAPHYRDTMNINALARDSLINANGIIERSFLPSKYAVEMSSAVYKYWVFTDQALPNDLIKRNMAVKDSSSPYGLRLLIEDYPYAVDGLEIWTAIKTWVQDYVSLYYATDNDIKNDSELQHWWKEVVEKGHGDLKDKPWWPKLQTFDELVEVCTIIIWTASALHAAVNFGQYPYGGLILNRPTLSRRLLPEEGTAEYDEMVKSSQKAYLRTITPKFQTLIDLSVIEILSRHASDEVYLGQRENPHWTSDSKALQAFQKFGNKLAEIEAKLTNKNNDPSLYHRVGPVQLPYTLLHPSSKEGLTFRGIPNSISI

SEQ ID NO: 8 - protein, 358 aa, synthetic construct
MFPNVTGLLNKGHKIRGTVVLMRKNVLDFNTIVSIGGGNVHGVIDSGINIIGSTLDGLTAFLGRSVSLQLISATKSDANGKGKVGKDTFLEGVLASLPTLGAGESAFNIHFEWDHEMGIPGAFYIKNYMQVEFFLKSLTLEDVPNHGTIRFVCNSWVYNSKLYKSPRIFFANKSYLPSETPSPLVKYREEELQTLRGDGTGERKLHERIYDYDVYNDLGNPDHGEHLARPILGGSSTHPYPRRGRTGRYPTRKDPNSEKPATETYVPRDENFGHLKSSDFLAYGIKSVSQCVVPAFESAFDLNFTPNEFDSFQDVRNLFEGGIKLPLDVISTISPLPVVKEIFRTDGEVYTTTCHSSE

SEQ ID NO: 9 - protein, 359 aa, synthetic construct
MFPNVTGLLNKGHKIRGTVVLMRKNVLDFNTIVSIGGGNVHGVIDSGINIIGSTLDGLTAFLGRSVSLQLISATKSDANGKGKVGKDTFLEGVLASLPTLGAGESAFNIHFEWDHEMGIPGAFYIKNYMQVEFFLKSLTLEDVPNHGTIRFVCNSWVYNSKLYKSPRIFFANKSYLPSETPSPLVKYREEELQTLRGDGTGERKLHERIYDYDVYNDLGNPDHGEHLARPILGGSSTHPYPRRGRTGRYPTRKDPNSEKPATETYVPRDENFGHLKSSDFLAYGIKSVSQCVVPAFESAFDLNFTPNEFDSFQDVRNLFEGGIKLPLDVISTISPLPVVKEIFRTDGEQVYTTTCHSSE

SEQ ID NO: 10 - DNA, 3541 bp, Pisum sativum
  source 1..3541: mol_type = genomic DNA
atgtttccaaatgtgacaggactcctaaacaagggccacaagataagagggacagtggtgttgatgcgtaagaatgtgttggacttcaacacaattgtgagtattggtggtggaaacgtccatggtgtcatcgactccggcatcaatatcattggttcaactcttgatggccttactgccttcttaggccgcagtgtctccctccagctgatcagtgctaccaagtctgatggttagttcattcatttcttcttacttttatatatctagacatgtagcacttttatatctagacatgtatcatcagaatcatcttaagtttttggaactcctgaataggagtttttggaactcctgtataggttagttgcagtttaacttcgacagaatatagaagttctatttaatcacgatttatttgtgataaatattttatgagaccctacttaactgtgacaaattttggacttatgcggtagcctgccttgcacggtcctgatcaggagtatagtcactaaatgaatgaatttaatgatatgtatgtagcaaatggaaaaggaaaagttggaaaggatacatttcttgaaggtgttcttgcttcattaccaaccttgggagctggtgaatctgcattcaatattcattttgaatgggatcatgaaatgggaattccaggtgcattttacatcaaaaactatatgcaagttgagttttttctcaagagtttaactctagaagatgttccaaatcatggaaccattcgcttcgtatgcaactcttgggtttacaactcaaaactctacaaaagtcctcgcattttcttcgccaacaaggtaatttatttacatctatatgttagtgcgtggtttgttagaatcacgatgaatcattatgattttacttgattgttatcatgcagtcatatcttccgagtgaaacaccgtctccacttgtcaagtatagagaagaagaattgcaaactctaagaggagatggaacaggagagcgcaagttacatgaaagaatctacgattatgatgtctataacgatttaggcaatccagatcacggcgaacatcttgctcgacctattctcggagggtctagcacacatccctatcctcgtaggggtaggaccggtcgatatccaacgaggaaaggttagatacaagaacatggtacaaataatgataaaaatgagataaactaaccttaagctattgtgttcttttgaacagatccgaatagcgagaaaccagctacagaaacatatgttccaagagatgaaaattttggtcacttgaagtcctctgactttcttgcatatggaataaaatctgtatctcaatgtgtagtacctgcatttgaatctgcatttgatttgaattttacgcctaacgagtttgatagttttcaagatgtgcgcaacctcttcgaaggtggaattaagcttcctctagatgtaattagcacaattagtcctttacctgtggtcaaagaaatctttcgtaccgatggtgaacaagtcctcaagtttacaccaccacatgtcattcgaggtccgacaaaatacatattataacgtaaattatactacgctttgagtaatttcattttattataagtttctttgtttttcgcagtgagtaagtctgcgtggatgaccgatgaagaatttgcacgagaaatgctcgctggcgtaaatccatgcatgatccgcggtcttcaagtaagttcaagaatttattttaatatttcggagaaatttttgctattcacattaaataattgcctatctaattgtaggagtttcctccgaaaagcaatctagatcccgcagaatacggtgatcacactagtaagatatctgtagatgtcttgaaccttgatggttgcaccatagatgaggtaaacacataataatcataattaaaaaaatatgattttggacttaatttggtgacataaagtgattaagattaattttacatggaacaggcacttgctagtgggagattatttatactagattatcatgacacatttattccatttctgagaaggatcaatgagacttctgcaaaagcttatgctactagaacaatactttttctaaaagaaaatggaactttaaagccagtggccattgaattaagtttgccacatcctgatggtgacaaatcaggttttgttagtaaagttatcttacctgcagatgaaggtgttgagagtactatttggcttctagcaaaagcttatgtggtggttaacgattcttgctatcatcaactcatgagccattggtaaaatatagtaatgtttatattttcacgtatgtgcgctctaaaatatcaggtaggatttgaactctgaatatatgtattacttattgatgatttttatatatttattggaataggttgaatactcatgcggtaattgagccgtttgttatagcaacaaatcggcagcttagtgtggttcatccgattaataaacttttagctcctcattatcgcgacacgatgaacatcaatgcacttgcaagagattctctaattaatgccaatggcataatagagagaagttttttgccttcgaagtatgctgtggaaatgtcttcagcggtttacaagtattgggttttcacagatcaagctctgccgaatgatcttatcaagaggtaatagaatgatcataggttcgaaaaatgaacaatctttttaaagatgattctatatttaaggactgagatagtaacatgtatatgcattttgcttgattcagaaacatggcagttaaagattcgtcgtctccatatggactccgtcttctgatagaggactacccgtacgctgttgacggattagagatatggacggctattaaaacatgggtccaagattatgtctcgctgtattatgcaacggacaatgatatcaaaaatgattccgagcttcaacattggtggaaagaggttgtagagaaaggtcatggtgacttgaaagataagccatggtggcctaagttgcaaacgtttgacgagctcgttgaagtttgcactatcatcatatggacggcttctgctctccacgcagctgttaattttggacaatatccttacggcggtttaattctaaaccgtccaactctgagtagaagattgcttcctgaggaaggaactgcggaatacgatgaaatggtgaagagttctcaaaaggcttacttgaggacaatcacgccgaagtttcagactcttattgatctttcagtgatagaaatattgtcaagacatgcttctgatgaagtctatctcggacaaagggaaaatccacactggacatctgattctaaagctttacaagcatttcaaaagtttggaaataaattggcggaaattgaggccaaacttacgaataagaacaatgatccgagcctgtaccatcgggtcgggccggttcaattgccatacactttgcttcatcccagcagcaaggaagggttaacctttagaggaattcctaatagtatctctatttaa

SEQ ID NO: 11 - DNA, 3530 bp, synthetic construct
  source 1..3530: mol_type = other DNA
atgtttccaaatgtgacaggactcctaaacaagggccacaagataagagggacagtggtgttgatgcgtaagaatgtgttggacttcaacacaattgtgagtattggtggtggaaacgtccatggtgtcatcgactccggcatcaatatcattggttcaactcttgatggccttactgccttcttaggccgcagtgtctccctccagctgatcagtgctaccaagtctgatggttagttcattcatttcttcttacttttatatatctagacatgtagcacttttatatctagacatgtatcatcagaatcatcttaagtttttggaactcctgaataggagtttttggaactcctgtataggttagttgcagtttaacttcgacagaatatagaagttctatttaatcacgatttatttgtgataaatattttatgagaccctacttaactgtgacaaattttggacttatgcggtagcctgccttgcacggtcctgatcaggagtatagtcactaaatgaatgaatttaatgatatgtatgtagcaaatggaaaaggaaaagttggaaaggatacatttcttgaaggtgttcttgcttcattaccaaccttgggagctggtgaatctgcattcaatattcattttgaatgggatcatgaaatgggaattccaggtgcattttacatcaaaaactatatgcaagttgagttttttctcaagagtttaactctagaagatgttccaaatcatggaaccattcgcttcgtatgcaactcttgggtttacaactcaaaactctacaaaagtcctcgcattttcttcgccaacaaggtaatttatttacatctatatgttagtgcgtggtttgttagaatcacgatgaatcattatgattttacttgattgttatcatgcagtcatatcttccgagtgaaacaccgtctccacttgtcaagtatagagaagaagaattgcaaactctaagaggagatggaacaggagagcgcaagttacatgaaagaatctacgattatgatgtctataacgatttaggcaatccagatcacggcgaacatcttgctcgacctattctcggagggtctagcacacatccctatcctcgtaggggtaggaccggtcgatatccaacgaggaaaggttagatacaagaacatggtacaaataatgataaaaatgagataaactaaccttaagctattgtgttcttttgaacagatccgaatagcgagaaaccagctacagaaacatatgttccaagagatgaaaattttggtcacttgaagtcctctgactttcttgcatatggaataaaatctgtatctcaatgtgtagtacctgcatttgaatctgcatttgatttgaattttacgcctaacgagtttgatagttttcaagatgtgcgcaacctcttcgaaggtggaattaagcttcctctagatgtaattagcacaattagtcctttacctgtggtcaaagaaatctttcgtaccgatggtgaagtttacaccaccacatgtcattcgaggtccgacaaaatacatattataacgtaaattatactacgctttgagtaatttcattttattataagtttctttgtttttcgcagtgagtaagtctgcgtggatgaccgatgaagaatttgcacgagaaatgctcgctggcgtaaatccatgcatgatccgcggtcttcaagtaagttcaagaatttattttaatatttcggagaaatttttgctattcacattaaataattgcctatctaattgtaggagtttcctccgaaaagcaatctagatcccgcagaatacggtgatcacactagtaagatatctgtagatgtcttgaaccttgatggttgcaccatagatgaggtaaacacataataatcataattaaaaaaatatgattttggacttaatttggtgacataaagtgattaagattaattttacatggaacaggcacttgctagtgggagattatttatactagattatcatgacacatttattccatttctgagaaggatcaatgagacttctgcaaaagcttatgctactagaacaatactttttctaaaagaaaatggaactttaaagccagtggccattgaattaagtttgccacatcctgatggtgacaaatcaggttttgttagtaaagttatcttacctgcagatgaaggtgttgagagtactatttggcttctagcaaaagcttatgtggtggttaacgattcttgctatcatcaactcatgagccattggtaaaatatagtaatgtttatattttcacgtatgtgcgctctaaaatatcaggtaggatttgaactctgaatatatgtattacttattgatgatttttatatatttattggaataggttgaatactcatgcggtaattgagccgtttgttatagcaacaaatcggcagcttagtgtggttcatccgattaataaacttttagctcctcattatcgcgacacgatgaacatcaatgcacttgcaagagattctctaattaatgccaatggcataatagagagaagttttttgccttcgaagtatgctgtggaaatgtcttcagcggtttacaagtattgggttttcacagatcaagctctgccgaatgatcttatcaagaggtaatagaatgatcataggttcgaaaaatgaacaatctttttaaagatgattctatatttaaggactgagatagtaacatgtatatgcattttgcttgattcagaaacatggcagttaaagattcgtcgtctccatatggactccgtcttctgatagaggactacccgtacgctgttgacggattagagatatggacggctattaaaacatgggtccaagattatgtctcgctgtattatgcaacggacaatgatatcaaaaatgattccgagcttcaacattggtggaaagaggttgtagagaaaggtcatggtgacttgaaagataagccatggtggcctaagttgcaaacgtttgacgagctcgttgaagtttgcactatcatcatatggacggcttctgctctccacgcagctgttaattttggacaatatccttacggcggtttaattctaaaccgtccaactctgagtagaagattgcttcctgaggaaggaactgcggaatacgatgaaatggtgaagagttctcaaaaggcttacttgaggacaatcacgccgaagtttcagactcttattgatctttcagtgatagaaatattgtcaagacatgcttctgatgaagtctatctcggacaaagggaaaatccacactggacatctgattctaaagctttacaagcatttcaaaagtttggaaataaattggcggaaattgaggccaaacttacgaataagaacaatgatccgagcctgtaccatcgggtcgggccggttcaattgccatacactttgcttcatcccagcagcaaggaagggttaacctttagaggaattcctaatagtatctctatttaa

SEQ ID NO: 12 - DNA, 3533 bp, synthetic construct
  source 1..3533: mol_type = other DNA
atgtttccaaatgtgacaggactcctaaacaagggccacaagataagagggacagtggtgttgatgcgtaagaatgtgttggacttcaacacaattgtgagtattggtggtggaaacgtccatggtgtcatcgactccggcatcaatatcattggttcaactcttgatggccttactgccttcttaggccgcagtgtctccctccagctgatcagtgctaccaagtctgatggttagttcattcatttcttcttacttttatatatctagacatgtagcacttttatatctagacatgtatcatcagaatcatcttaagtttttggaactcctgaataggagtttttggaactcctgtataggttagttgcagtttaacttcgacagaatatagaagttctatttaatcacgatttatttgtgataaatattttatgagaccctacttaactgtgacaaattttggacttatgcggtagcctgccttgcacggtcctgatcaggagtatagtcactaaatgaatgaatttaatgatatgtatgtagcaaatggaaaaggaaaagttggaaaggatacatttcttgaaggtgttcttgcttcattaccaaccttgggagctggtgaatctgcattcaatattcattttgaatgggatcatgaaatgggaattccaggtgcattttacatcaaaaactatatgcaagttgagttttttctcaagagtttaactctagaagatgttccaaatcatggaaccattcgcttcgtatgcaactcttgggtttacaactcaaaactctacaaaagtcctcgcattttcttcgccaacaaggtaatttatttacatctatatgttagtgcgtggtttgttagaatcacgatgaatcattatgattttacttgattgttatcatgcagtcatatcttccgagtgaaacaccgtctccacttgtcaagtatagagaagaagaattgcaaactctaagaggagatggaacaggagagcgcaagttacatgaaagaatctacgattatgatgtctataacgatttaggcaatccagatcacggcgaacatcttgctcgacctattctcggagggtctagcacacatccctatcctcgtaggggtaggaccggtcgatatccaacgaggaaaggttagatacaagaacatggtacaaataatgataaaaatgagataaactaaccttaagctattgtgttcttttgaacagatccgaatagcgagaaaccagctacagaaacatatgttccaagagatgaaaattttggtcacttgaagtcctctgactttcttgcatatggaataaaatctgtatctcaatgtgtagtacctgcatttgaatctgcatttgatttgaattttacgcctaacgagtttgatagttttcaagatgtgcgcaacctcttcgaaggtggaattaagcttcctctagatgtaattagcacaattagtcctttacctgtggtcaaagaaatctttcgtaccgatggtgaacaagtttacaccaccacatgtcattcgaggtccgacaaaatacatattataacgtaaattatactacgctttgagtaatttcattttattataagtttctttgtttttcgcagtgagtaagtctgcgtggatgaccgatgaagaatttgcacgagaaatgctcgctggcgtaaatccatgcatgatccgcggtcttcaagtaagttcaagaatttattttaatatttcggagaaatttttgctattcacattaaataattgcctatctaattgtaggagtttcctccgaaaagcaatctagatcccgcagaatacggtgatcacactagtaagatatctgtagatgtcttgaaccttgatggttgcaccatagatgaggtaaacacataataatcataattaaaaaaatatgattttggacttaatttggtgacataaagtgattaagattaattttacatggaacaggcacttgctagtgggagattatttatactagattatcatgacacatttattccatttctgagaaggatcaatgagacttctgcaaaagcttatgctactagaacaatactttttctaaaagaaaatggaactttaaagccagtggccattgaattaagtttgccacatcctgatggtgacaaatcaggttttgttagtaaagttatcttacctgcagatgaaggtgttgagagtactatttggcttctagcaaaagcttatgtggtggttaacgattcttgctatcatcaactcatgagccattggtaaaatatagtaatgtttatattttcacgtatgtgcgctctaaaatatcaggtaggatttgaactctgaatatatgtattacttattgatgatttttatatatttattggaataggttgaatactcatgcggtaattgagccgtttgttatagcaacaaatcggcagcttagtgtggttcatccgattaataaacttttagctcctcattatcgcgacacgatgaacatcaatgcacttgcaagagattctctaattaatgccaatggcataatagagagaagttttttgccttcgaagtatgctgtggaaatgtcttcagcggtttacaagtattgggttttcacagatcaagctctgccgaatgatcttatcaagaggtaatagaatgatcataggttcgaaaaatgaacaatctttttaaagatgattctatatttaaggactgagatagtaacatgtatatgcattttgcttgattcagaaacatggcagttaaagattcgtcgtctccatatggactccgtcttctgatagaggactacccgtacgctgttgacggattagagatatggacggctattaaaacatgggtccaagattatgtctcgctgtattatgcaacggacaatgatatcaaaaatgattccgagcttcaacattggtggaaagaggttgtagagaaaggtcatggtgacttgaaagataagccatggtggcctaagttgcaaacgtttgacgagctcgttgaagtttgcactatcatcatatggacggcttctgctctccacgcagctgttaattttggacaatatccttacggcggtttaattctaaaccgtccaactctgagtagaagattgcttcctgaggaaggaactgcggaatacgatgaaatggtgaagagttctcaaaaggcttacttgaggacaatcacgccgaagtttcagactcttattgatctttcagtgatagaaatattgtcaagacatgcttctgatgaagtctatctcggacaaagggaaaatccacactggacatctgattctaaagctttacaagcatttcaaaagtttggaaataaattggcggaaattgaggccaaacttacgaataagaacaatgatccgagcctgtaccatcgggtcgggccggttcaattgccatacactttgcttcatcccagcagcaaggaagggttaacctttagaggaattcctaatagtatctctatttaa

SEQ ID NO: 13 - DNA, 21 bp, synthetic construct
  source 1..21: mol_type = other DNA
tggtcacttgaagtcctctga

SEQ ID NO: 14 - DNA, 20 bp, synthetic construct
  source 1..20: mol_type = other DNA
ggttcaagacatctacagat

SEQ ID NO: 15 - DNA, 24 bp, synthetic construct
  source 1..24: mol_type = other DNA
gtaccgatggtgaacaagtcctca

SEQ ID NO: 16 - protein, 1264 aa, synthetic construct
MLFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHRNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNR

SEQ ID NO: 17 - DNA, 55 bp, synthetic construct
  source 1..55: mol_type = other DNA
gaaatctttcgtaccgatggtgaacaagtcctcaagtttacaccaccacatgtca

SEQ ID NO: 18 - DNA, 47 bp, synthetic construct
  source 1..47: mol_type = other DNA
gaaatctttcgtaccgatggtgaacaagtttacaccaccacatgtca

SEQ ID NO: 19 - DNA, 47 bp, synthetic construct
  source 1..47: mol_type = other DNA
gaaatctttcgtaccgatggtgatcaagtttacaccaccacatgtca

SEQ ID NO: 20 - DNA, 43 bp, synthetic construct
  source 1..43: mol_type = other DNA
gaaatctttcgtaccgatggtgagtttacaccaccacatgtca

SEQ ID NO: 21 - DNA, 45 bp, synthetic construct
  source 1..45: mol_type = other DNA
gaaatctttcgtaccgatggtgaaagtttacaccaccacatgtca

SEQ ID NO: 22 - DNA, 324 bp, Pisum sativum
  source 1..324: mol_type = genomic DNA
atcctaatagtgagagtagaagtgattatgtgtatcttccaagagatgaagcttttggtcacttgaagtcatcagattttctcacttatggattaaaagctgtgtctcaaaacgtggttcctgcattagaatctgtgttctttgatttgaatttcacgccaaatgagtttgatagctttgatgaagttcatggattgtatgagggtggaattaagctgccaacaaatatacttagccagataagcccgttacccgtgcttaaggagatctttcgaaccgatggtgaaaatacccttaaatatccaccacctaaagtaattcaag

SEQ ID NO: 23 - DNA, 24 bp, synthetic construct
  source 1..24: mol_type = other DNA
tgttctttgatttgaatttcacgc

SEQ ID NO: 24 - DNA, 296 bp, synthetic construct
  source 1..296: mol_type = other DNA
atcctaatagtgagagtagaagtgattatgtgtatcttccaagagatgaagcttttggtcacttgaagtcatcagattttctcacttatggattaaaagctgtgtctcaaaacgtggttcctgcattagaatctgtgagtttgatagctttgatgaagttcatggattgtatgagggtggaattaagctgccaacaaatatacttagccagataagcccgttacccgtgcttaaggagatctttcgaaccgatggtgaaaatacccttaaatatccaccacctaaagtaattcaag

SEQ ID NO: 25 - protein, 861 aa, Pisum sativum
MFSGVTGILNRGHKIKGTVVLMRKNVLDINSLTTVGGVIGQGFDILGSTVDNLTAFLGRSVSLQLISATKPDATGKGKLGKATFLEGIISSLPTLGAGQSAFKIHFEWDDDMGIPGAFYIKNFMQTEFFLVSLTLDDIPNHGSIYFVCNSWIYNAKHHKIDRIFFANQTYLPSETPAPLVHYREEELNNLRGDGTGERKEWERIYDYDVYNDLGNPDSGENHARPVLGGSETYPYPRRGRTGRKPTRKDPNSESRSDYVYLPRDEAFGHLKSSDFLTYGLKAVSQNVVPALESVFFDLNFTPNEFDSFDEVHGLYEGGIKLPTNILSQISPLPVLKEIFRTDGENTLKYPPPKVIQVSRSGWMTDEEFAREMLAGVNPNVICCLQEFPPRSKLDSQIYGDHTSKISKEHLEPNLEGLTVEEAIQNKKLFLLDHHDSIMPYLRRINSTSTKAYATRTILFLNNNQNLKPLAIELSLPHPQGDEHGAVSYVYQPALEGVESNIWLLAKAYVIVNDSCYHQLVSHWLNTHAVVEPFVIATNRHLSCLHPIYKLLYPHYRDTMNINSLARLSLVNDGGIIEKTFLWGRYSMEMSSKVYKNWVFTEQALPADLIKRGMAIEDPSSPCGVKLVVEDYPYAVDGLEIWAIIKTWVQDYVSLYYTSDEKLRQDSELQAWWKELVEVGHGDKKNEPWWPKMQTREDLIEVCSIVIWTASALHAAVNFGQYSYGGLILNRPTLSRRFMPEKGSAEFEELVKSPQKAYLKTITPKFQTLIDLSVIEILSRHASDELYLGERDNPNWTSDKRALEAFKKFGNKLAEIEKKLTQRNNDEKLRNRHGPVEMPYTLLYPSSKEGLTFRGIPNSISI

SEQ ID NO: 26 - protein, 319 aa, synthetic construct
MFSGVTGILNRGHKIKGTVVLMRKNVLDINSLTTVGGVIGQGFDILGSTVDNLTAFLGRSVSLQLISATKPDATGKGKLGKATFLEGIISSLPTLGAGQSAFKIHFEWDDDMGIPGAFYIKNFMQTEFFLVSLTLDDIPNHGSIYFVCNSWIYNAKHHKIDRIFFANQTYLPSETPAPLVHYREEELNNLRGDGTGERKEWERIYDYDVYNDLGNPDSGENHARPVLGGSETYPYPRRGRTGRKPTRKDPNSESRSDYVYLPRDEAFGHLKSSDFLTYGLKAVSQNVVPALESVSLIALMKFMDCMRVELSCQQIYLAR

SEQ ID NO: 27 - DNA, 3354 bp, Pisum sativum
  source 1..3354: mol_type = genomic DNA
atgttttcaggcgtgactggtattctgaatagaggccataaaataaaagggacagtggtgttgatgagaaagaatgtgttggatataaacagcttaaccactgttggtggtgttatcggtcaaggcttcgacattctcggttccacggttgataatctcactgctttcttaggccgttctgtttctcttcagttgatcagtgcaacaaaacctgatggtacagtttctataacattttgagcttttgaaaatgaattttgattctgtatttaattgtttttatttgtattttgttagcgactgggaaagggaagcttggaaaagctacttttctggaaggtataatttcttcattaccaactttgggagctggacaatctgcttttaagattcactttgaatgggatgatgatatgggaattcctggtgcattttatattaaaaactttatgcaaactgagttttttcttgtgagtttgactcttgatgacattccaaatcacggaagtatctactttgtatgcaactcttggatatataatgccaaacaccacaaaatcgatcgcattttctttgcgaatcaggtatatatatgtttatatttgtatatttgttttcaactttaaggcttatgaggaagataatcgaagaattgtgttgcagacgtatcttccgagtgaaactccggctccattagtccattatagagaagaagaattgaataatttaagaggagacggaacaggagagcgcaaagaatgggaaaggatctatgattacgatgtctataatgatttaggaaatccagattcgggtgagaaccatgctcgtccggttcttggagggtctgaaacctatccttatccgcgtagggggagaaccggtcgaaaaccaaccagaaaaggtttaacttctattacattgagtatgaataatgttttatcagttatcactatacaatatcatttcaactattttattgctattaagcagatcctaatagtgagagtagaagtgattatgtgtatcttccaagagatgaagcttttggtcacttgaagtcatcagattttctcacttatggattaaaagctgtgtctcaaaacgtggttcctgcattagaatctgtgttctttgatttgaatttcacgccaaatgagtttgatagctttgatgaagttcatggattgtatgagggtggaattaagctgccaacaaatatacttagccagataagcccgttacccgtgcttaaggagatctttcgaaccgatggtgaaaatacccttaaatatccaccacctaaagtaattcaaggtatttcattttcataccttaaaaatactcatgcctaaaatcgccatgataagcgcttatactataagctgcaaaaagtatttttatgtaaacatgatcttactagtctatattattgattgaacagtaagtaggtctggatggatgactgatgaagaattcgcaagagaaatgcttgctggagtaaatccaaatgtgatatgctgtcttcaggtagacgtagatacaattggccgagcattgttactaaatatcgcgttttttttgtttctttattcttttgaaccggattgttggtctgacagacgacctaatgaaggatcgttgtttgattcttgcaggagtttcctccgcgaagcaagcttgacagtcaaatctatggtgatcacactagtaaaataagcaaagagcacctagagcctaatttagaaggactcacagtagaagaggtacacttttgcttgatgagttctattattttctatgctttgttaagttttattattatgattaacaatgagaattgtatttgattactgctgtaaaggcaattcaaaacaagaaattgttcctactagatcatcatgactcaattatgccatacttaaggagaataaactcaacttccacaaaggcttatgctaccagaacaatccttttcttgaacaataaccaaaatttgaagcccctggctatagagttgagtttgccacaccctcaaggagatgaacacggcgctgttagttatgtttatcaacctgcgctagaaggcgttgaaagtaacatttggttattggctaaagcttatgtgattgtgaatgattcctgctaccatcaacttgttagccattggtatatattttgtcgataaattatataaattataacttttgatatgtatatatatcgatgaattgattcgcgattcttgatgataataggttgaatactcacgcagttgttgagccgtttgtcatagcaactaacaggcatctgagttgtcttcacccgatttataaacttttgtatcctcactatcgcgatacaatgaatataaactctctcgctcgactttccctcgtcaacgacggaggtatcatcgagaaaacttttttgtggggaagatattctatggaaatgtcttctaaagtttacaagaattgggttttcacagagcaagcattgcctgctgatcttataaaaaggtaacatttttacacaaacataagacataaacttaaagaactttctatgttgattataatgtgaaattgttgttttagaggaatggctattgaggatccatcttctccatgtggtgttaagcttgtggtagaggattatccttatgctgttgatggattagagatatgggctataataaagacatgggttcaagactatgtctccttgtactatacctcagatgaaaaacttaggcaagattctgaacttcaagcttggtggaaagagctcgtggaggtcggtcatggtgacaagaaaaacgagccgtggtggcctaagatgcaaactcgagaagacttaattgaagtttgcagtattgtcatatggactgcttcagctcttcacgcagctgttaatttcgggcaatattcctacggaggtttaatcttaaaccgtccaactcttagtagacgattcatgcctgagaaaggttcagctgagttcgaggagctagtgaaaagtcctcaaaaggcttacttgaagacaattacgccgaagtttcagactcttattgatctttctgttatagaaatattgtcgaggcatgcttccgatgagttgtaccttggggagagagacaatccgaattggacatcggataaaagggcattagaggcttttaagaagtttggaaacaagttggctgaaattgagaagaaactcactcagagaaacaatgatgagaagctaagaaatcgtcatggaccggttgagatgccttatactttgctctatccttctagtaaagaaggactaacttttagaggaattcctaatagtatctccatctga

SEQ ID NO: 28 - DNA, 1208 bp, synthetic construct
  source 1..1208: mol_type = other DNA
atgttttcaggcgtgactggtattctgaatagaggccataaaataaaagggacagtggtgttgatgagaaagaatgtgttggatataaacagcttaaccactgttggtggtgttatcggtcaaggcttcgacattctcggttccacggttgataatctcactgctttcttaggccgttctgtttctcttcagttgatcagtgcaacaaaacctgatggtacagtttctataacattttgagcttttgaaaatgaattttgattctgtatttaattgtttttatttgtattttgttagcgactgggaaagggaagcttggaaaagctacttttctggaaggtataatttcttcattaccaactttgggagctggacaatctgcttttaagattcactttgaatgggatgatgatatgggaattcctggtgcattttatattaaaaactttatgcaaactgagttttttcttgtgagtttgactcttgatgacattccaaatcacggaagtatctactttgtatgcaactcttggatatataatgccaaacaccacaaaatcgatcgcattttctttgcgaatcaggtatatatatgtttatatttgtatatttgttttcaactttaaggcttatgaggaagataatcgaagaattgtgttgcagacgtatcttccgagtgaaactccggctccattagtccattatagagaagaagaattgaataatttaagaggagacggaacaggagagcgcaaagaatgggaaaggatctatgattacgatgtctataatgatttaggaaatccagattcgggtgagaaccatgctcgtccggttcttggagggtctgaaacctatccttatccgcgtagggggagaaccggtcgaaaaccaaccagaaaaggtttaacttctattacattgagtatgaataatgttttatcagttatcactatacaatatcatttcaactattttattgctattaagcagatcctaatagtgagagtagaagtgattatgtgtatcttccaagagatgaagcttttggtcacttgaagtcatcagattttctcacttatggattaaaagctgtgtctcaaaacgtggttcctgcattagaatctgtgagtttgatagctttgatgaagttcatggattgtatgagggtggaattaagctgccaacaaatatacttagccagataa

SEQ ID NO: 29 - DNA, 1158 bp, Pisum sativum
  source 1..1158: mol_type = genomic DNA
atgggagcaggtggcagaacctctcttccaccctcgcaaacaaaaccagaacaagatcaacatttgaagcgtgtcccaattaccaaaccacccttcactctcggagaagttaagaaagcaattccaccacattgtttcaaccgttccttaattcgatcattttcctatgtcattgtcgacctcaccatagcctcaatccttgcctacctagccataagctacattcctacccttcccggtaacaactatctttggacatggcccatttactgggccgtccaaggatgtgtccttaccggtgtttgggtcgttgcacacgaatgcgggcatcatgcattcagtaattaccaatgggttgatgacttagttgggctcatccttcattcatgtctcttggtaccatatttctcatggaagtatagtcaccgtcgccaccattcgaacactgggtccctagaacgcgacgaggtttttgtccctaagaaaaaatctcaaattggttggtactataaatacctcgacaacccactaggaagatttctcacactggctgtaacactcactcttggttggccgctttacctagccttcaacgtttccggccgatactacgataggttcgcttcacattatgatccatacggcccaatttactccgatcgcgaaagacttcaaatctatgtttcagacgcaggcgttctcgcagtttcttacgggctttaccgtcttgttttggcacaaggtttgaattgggttattcgagtttacggagttccacttcttatagtaaacggatttctagttatgatcacttacttgcaacacactcatcctgcactgcctcattatgattcaagtgaatgggattggatgagaggtgctttggcaactgtggatagagattatggaattcttaacaaagtttttcataacataactgatacccatgttgttcatcatatgttttcaactatgccacattatcatgctatggaagctacaaaagctgtgaaacctattttgggcgagtattatcattttgatgggactccagtttataaggctatgtggagagaggctaaggagtgtctttatgttgatccagatgaagggagtcagattaaaggtgtttattggtatagaaacaaacttgagtaa

SEQ ID NO: 30 - DNA, 24 bp, synthetic construct
  source 1..24: mol_type = other DNA
acatttgaagcgtgtcccaattac

SEQ ID NO: 31 - DNA, 105 bp, synthetic construct
  source 1..105: mol_type = other DNA
atgggagcaggtggcagaacctctcttccaccctcgcaaacaaaaccagaacaagatcaagcgtgtcccaattaccaaaccacccttcactctcggagaagttaa

SEQ ID NO: 32 - DNA, 111 bp, synthetic construct
  source 1..111: mol_type = other DNA
atgggagcaggtggcagaacctctcttccaccctcgcaaacaaaaccagaacaagatcaatttgaagcgtgtcccaattaccaaaccacccttcactctcggagaagttaa

SEQ ID NO: 33 - protein, 385 aa, Pisum sativum
MGAGGRTSLPPSQTKPEQDQHLKRVPITKPPFTLGEVKKAIPPHCFNRSLIRSFSYVIVDLTIASILAYLAISYIPTLPGNNYLWTWPIYWAVQGCVLTGVWVVAHECGHHAFSNYQWVDDLVGLILHSCLLVPYFSWKYSHRRHHSNTGSLERDEVFVPKKKSQIGWYYKYLDNPLGRFLTLAVTLTLGWPLYLAFNVSGRYYDRFASHYDPYGPIYSDRERLQIYVSDAGVLAVSYGLYRLVLAQGLNWVIRVYGVPLLIVNGFLVMITYLQHTHPALPHYDSSEWDWMRGALATVDRDYGILNKVFHNITDTHVVHHMFSTMPHYHAMEATKAVKPILGEYYHFDGTPVYKAMWREAKECLYVDPDEGSQIKGVYWYRNKLE

SEQ ID NO: 34 - protein, 34 aa, synthetic construct
MGAGGRTSLPPSQTKPEQDQACPNYQTTLHSRRS

SEQ ID NO: 35 - protein, 36 aa, synthetic construct
MGAGGRTSLPPSQTKPEQDQFEACPNYQTTLHSRRS

SEQ ID NO: 36 - DNA, 1158 bp, Pisum sativum; identical to SEQ ID NO: 29
  source 1..1158: mol_type = genomic DNA

SEQ ID NO: 37 - DNA, 105 bp, synthetic construct; identical to SEQ ID NO: 31
  source 1..105: mol_type = other DNA

SEQ ID NO: 38 - DNA, 111 bp, synthetic construct; identical to SEQ ID NO: 32
  source 1..111: mol_type = other DNA

SEQ ID NO: 39 - DNA, 90 bp, Pisum sativum
  source 1..90: mol_type = genomic DNA
tggccatggaagcttttcaaatagtgttaagctgaatagttttgtgggccatatcttgcattcttcgattcttgttccatatcatggatg

SEQ ID NO: 40 - DNA, 24 bp, synthetic construct
  source 1..24: mol_type = other DNA
aaatagtgttaagctgaatagttt

SEQ ID NO: 41 - DNA, 82 bp, synthetic construct
  source 1..82: mol_type = other DNA
tggccatggaagcttttcaaatagtgttaagctttgtgggccatatcttgcattcttcgattcttgttccatatcatggatg

SEQ ID NO: 42 - DNA, 46 bp, synthetic construct
  source 1..46: mol_type = other DNA
tgggccatatcttgcattcttcgattcttgttccatatcatggatg

SEQ ID NO: 43 - protein, 388 aa, Pisum sativum
MVVQEPQNLLHVGNGDVLVDDEKKHHHTSFDPSAPPPFKIAEVRAAIPKHCWVKNPWISLSYLLRDIFIVTALVAAAIHFNNWIFWPIYWISQGTMFWALFVLGHDCGHGSFSNSVKLNSFVGHILHSSILVPYHGWRISHRTHHQNHGHVEKDESWVPLTEKMYKSLDNMTKTMRFTFPFPIFAYPFYLWNRSPGKEGSHFNPYSKLFTPSERKDVLTSTFCWGIMFCGLVYLSLIKGPFFMLKIYGVPYWIFIMWLDFVTYMHHHGYSQKLPWYRGKEWDYLRGGLTTVDRDYGWVNNIHHDIGTHVIHHLFPQIPHYHLIEATEAAKPVLGKYYREPEKSGAVPFHLFKYFLHSMNQDHFVSDSGDIVFYQTDPKLHNNSWTKSE

SEQ ID NO: 44 - protein, 138 aa, synthetic construct
MVVQEPQNLLHVGNGDVLVDDEKKHHHTSFDPSAPPPFKIAEVRAAIPKHCWVKNPWISLSYLLRDIFIVTALVAAAIHFNNWIFWPIYWISQGTMFWALFVLGHDCGHGSFSNSVKLCGPYLAFFDSCSISWMENQP

SEQ ID NO: 45 - protein, 126 aa, synthetic construct
MVVQEPQNLLHVGNGDVLVDDEKKHHHTSFDPSAPPPFKIAEVRAAIPKHCWVKNPWISLSYLLRDIFIVTALVAAAIHFNNWIFWPIYWISQGTMFWALFVLGHDCGPYLAFFDSCSISWMENQP

SEQ ID NO: 46 - DNA, 2904 bp, Pisum sativum
  source 1..2904: mol_type = genomic DNA
atggttgttcaagaacctcaaaatctgctacatgttggtaatggtgatgttcttgttgatgatgaaaaaaaacaccaccatacaagttttgatccaagtgctcctccaccatttaagattgcagaggttcgtgccgcgattccgaaacactgttgggttaagaatccatggatttctcttagttatcttcttagagacattttcattgtcactgcattggtagctgctgcaattcatttcaataattggatcttttggccaatttattggatttctcaaggaactatgttttgggctctttttgttcttggacatgattggtattcttttttttcattcattttttaatttcaataatttttttatgttaatttaactcatagatttgttttttttttggtatctgtgtgtttgatttgaacagtggccatggaagcttttcaaatagtgttaagctgaatagttttgtgggccatatcttgcattcttcgattcttgttccatatcatggatggtttgttcttttttttaatatgattctttttaagatgcttttctgttcattttattctgtttaagattcttttttcttttttttgaatctgatttatttttgcaggagaatcagccatagaactcatcatcaaaatcatggtcatgttgagaaagatgaatcctgggttcctgtatataactttttctttcttttcatttaccatgaaatttacacaattaccgtaacaaaattacgatgattgtgtctatttcacggtataatcttaatttgtctaaaatggtgaaaatctttgttaattgtttatgatatcatgtttttgtgtttgtatgttgaatgtctcagttgacagagaagatgtacaagagtttagacaacatgacaaaaacaatgagatttacttttccttttccaatctttgcataccctttttatttggtgagtgattcccttacaacattgtttgttttgttttgttttgttacattataattataaataataatttctaacaattttttttatttatgtaaaccacatttcagtggaacagaagtcctggtaaagaaggttcacatttcaatccatatagcaaattgttcacaccaagtgagagaaaagatgtgttaacttcaacattttgttggggtatcatgttttgtgggcttgtttatttatccttaataaagggtcctttttttatgctcaaaatctatggagttccttattgggtaatattattattattatttaatatttatttcacattttcttttatgtttaaaattagtgtaaaagatttactttttttttcttctgatatgtagatctttatcatgtggttggactttgtcacatatatgcatcaccatggttactctcagaaactaccttggtatcgtggcaaggtaatataaaaatattattattcaaaaaaatttgtctctgttttcttggctgttattcggtgaaagagaatctctaaaagctaaggaattggactttagtttcagatttctttttctttttaaattcctttattaaatatatatttatattattaaccaagatattttgaaggtgaaataatataattataagcaattctgtgtacatgaaaagtatttaggaatttgttctaagaaaacatgttggatgtaagtttttaaataagggtcgcacattgatataaatagtatagatattatgatgctgattatggtaatgtgaaataaataataatttgttcttttatataaaaacggtgaataatgatatcagtgtcgtaataatattttgataattggacaggaatgggactatctaagaggtggtttaacgaccgtggatcgtgactatggttgggttaacaacattcaccatgacattggcacacatgttattcatcatctttttcctcaaattccacattatcatttgattgaagcggtacattctctttctttcactctttttgtttttttactttctcaataacaaaggaataaaggccccaaacaataaagaaaaaggcccacaattcaacttgcatagttacctttgaagtggagtgtgacaacacaaggactttgtctttaatgttagggttcatacactagacttaactccacttgcatgtttgtttaatttccattgaaagcaaacaaaaaaaccctcaaaatttgtcattgttgcatttttaccagattttacaatatagccaatctcaaccatttattttatatctaactattcagaattaaattcactctcatcatggtaaaatgaatccgacccattatacatatatattttgtctgtattctttacgggataaattaaagtttaagagctgtatcctctctatatcattctcgcaacgttttttctaaagacagattgatattttagactcacattctctaatatgataattttattccacccccattatttttagaaactttgcacgggtttggcacaatgttagttgcatgtttgaaagaaataatgataaaatataagatgcattaccatattacttttattaagggaagaaaaaatgaatgagaaagtgtggggtgtattaccaaaagaggaggtgtgaatagttaccgaatatagaatcagaaacaaacagtgtcattgaatcgtacaataaaacatgtttgtgtttgttttttcctgtgtgatttcagacagaagcagcaaagccagtgctaggaaaatattatcgtgagccagagaaatctggggcagtcccattccatctctttaagtactttctacacagcatgaatcaggaccacttcgtcagtgattctggagacattgttttctaccaaacagaccctaagctccataataattcttggaccaagtctgagtaa

SEQ ID NO: 47 - DNA, 626 bp, synthetic construct
  source 1..626: mol_type = other DNA
atggttgttcaagaacctcaaaatctgctacatgttggtaatggtgatgttcttgttgatgatgaaaaaaaacaccaccatacaagttttgatccaagtgctcctccaccatttaagattgcagaggttcgtgccgcgattccgaaacactgttgggttaagaatccatggatttctcttagttatcttcttagagacattttcattgtcactgcattggtagctgctgcaattcatttcaataattggatcttttggccaatttattggatttctcaaggaactatgttttgggctctttttgttcttggacatgattggtattcttttttttcattcattttttaatttcaataatttttttatgttaatttaactcatagatttgttttttttttggtatctgtgtgtttgatttgaacagtggccatggaagcttttcaaatagtgttaagctttgtgggccatatcttgcattcttcgattcttgttccatatcatggatggtttgttcttttttttaatatgattctttttaagatgcttttctgttcattttattctgtttaagattcttttttcttttttttgaatctgatttatttttgcaggagaatcagccatag

SEQ ID NO: 48 - DNA, 585 bp, synthetic construct
  source 1..585: mol_type = other DNA
atggttgttcaagaacctcaaaatctgctacatgttggtaatggtgatgttcttgttgatgatgaaaaaaaacaccaccatacaagttttgatccaagtgctcctccaccatttaagattgcagaggttcgtgccgcgattccgaaacactgttgggttaagaatccatggatttctcttagttatcttcttagagacattttcattgtcactgcattggtagctgctgcaattcatttcaataattggatcttttggccaatttattggatttctcaaggaactatgttttgggctctttttgttcttggacatgattggtattcttttttttcattcattttttaatttcaataatttttttatgttaatttaactcatagatttgttttttttttggtatctgtgtgtttgatttgtgggccatatcttgcattcttcgattcttgttccatatcatggatggtttgttcttttttttaatatgattctttttaagatgcttttctgttcattttattctgtttaagattcttttttcttttttttgaatctgatttatttttgcaggagaatcagccatag

SEQ ID NO: 49 - DNA, 67 bp, Pisum sativum
  source 1..67: mol_type = genomic DNA
gagaattagccataggacacatcatcaaaaccatggaaatgttgagaaggatgagtcatgggtgccg

SEQ ID NO: 50 - DNA, 24 bp, synthetic construct
  source 1..24: mol_type = other DNA
agtcatgggtgccggtatgtgaat

SEQ ID NO: 51 - DNA, 62 bp, synthetic construct
  source 1..62: mol_type = other DNA
gagaattagccataggacacatcatcaaaaccatggaaatgttgagaaggatgagggtgccg

SEQ ID NO: 52 - DNA, 29 bp, synthetic construct
  source 1..29: mol_type = other DNA
gagaattagccataggacacatcatcaaa

SEQ ID NO: 53 - protein, 387 aa, Pisum sativum
MAVTASIHNHLSQNQQTLGQETMAVSNGGADFDPSAPPPFKIADIRAAIPKHCWVKSTWRSLSYVVRDVLVVAALIVAAIRFDSWFFWPIYWPLQGTMFWAIFVLGHDCGHGSFSDSYFVNSLVGHVLHSSILVPYHGWRISHRTHHQNHGNVEKDESWVPLTEKVYKSLDSMTKFVRFTVPLPMFAYPIYLWRRSPGKSGSHFHPDSNLFSPNERKEVTLSTVCWFLMFSFLLYLSFTTSPILILKLYGVPYWIFVMWLDFVTYLHHHGYTQKLPWYRGQEWSYLRGGLTTVDRDYGWINNIHHDIGTHVVHHLFPQIPHYHLIEATKAAKPVFGKYYREPEKSSPIPFHLIKYLAQSMKHDHFVSDTGEILFYQTDPELNKNHHD

SEQ ID NO: 54 - protein, 205 aa, synthetic construct
MAVTASIHNHLSQNQQTLGQETMAVSNGGADFDPSAPPPFKIADIRAAIPKHCWVKSTWRSLSYVVRDVLVVAALIVAAIRFDSWFFWPIYWPLQGTMFWAIFVLGHDCGHGSFSDSYFVNSLVGHVLHSSILVPYHGWRISHRTHHQNHGNVEKDEGAVDRKGLQEFRQHDKVCEVHCSFTNVCISNLFVEKKSWKIRFSLSSR

SEQ ID NO: 55 - protein, 194 aa, synthetic construct
MAVTASIHNHLSQNQQTLGQETMAVSNGGADFDPSAPPPFKIADIRAAIPKHCWVKSTWRSLSYVVRDVLVVAALIVAAIRFDSWFFWPIYWPLQGTMFWAIFVLGHDCGHGSFSDSYFVNSLVGHVLHSSILVPYHGWRISHRTHHQIDRKGLQEFRQHDKVCEVHCSFTNVCISNLFVEKKSWKIRFSLSSR

SEQ ID NO: 56 - DNA, 2624 bp, Pisum sativum
  source 1..2624: mol_type = genomic DNA
atggctgtaacagcctctattcataatcacctatctcaaaaccaacaaacactaggccaagaaaccatggctgtctccaatggtggtgccgattttgaccctagtgctcctccaccgtttaagattgcagatattagagctgcaattccgaagcattgttgggtcaagagtacttggagatctctgagctatgttgttagagatgtcttagttgttgctgcattgattgttgctgcaattcggtttgatagctggtttttttggcctatttattggcctcttcaagggactatgttttgggcgatttttgttcttggacatgactggtaaaaatttatgttttttttttgtttttgttttgctcatgattatgccttttggttttgttcatgttttgaattgaatattgtgtttttgttttgaatagtggccatggaagcttctcagatagttattttgtgaatagcttggtgggccatgttttgcactcctcaattcttgttccataccatggatggttagtgaatttgttcttagaatctttttttattgttcattggtcaaatctgtaattgtgagtttgtcggatttacgatgatgacagattaaaacgtaatttttatcatttgattttgttaatttatctgttaattcgaacctgcactaaattcatgaagtgtactcaaatcaattttcttttgttcttatggttttttccaggagaattagccataggacacatcatcaaaaccatggaaatgttgagaaggatgagtcatgggtgccggtatgtgaattaaatgcttccatgtgttgtttttaattgatttaacatagtattcagaaccgtctcaaattttttaaggctttgaattgaataatcgagattcaattctgtcaaaattgatactataaattttatttaatcacggtttaattaagagattttttttttgaaatcttgtttaattactcagattttagacttatgcggcagtctgcttacacgccacaaatacgactttgattaaatgatgtaatctttaagaagttttttaaattaattatctgatttgttttttgtgtgtcattatcttagttgacagaaaaggtttacaagagtttagacagcatgacaaagtttgtgaggttcactgttcctttaccaatgtttgcatatccaatttatttggtgagtgattttagtttttaaattattagtactatttgaaaaaaaacataacagttgaagaatctgaattttaatattctgatgtgaaatttcacatttttcagtggagaagaagtcctggaaaatcaggttctcactttcatccagatagcaacttgttctcacccaatgagagaaaagaagtgacactttcaacagtgtgctggtttctcatgttttcttttcttctctatttgtccttcacaactagtccaattttaattctcaagctatatggtgttccctattgggtaaaaatttacccttgcacttttcatatttttatcataacttcttagtcaaattcattttttttttaaaaattgttgagttataaatatcaatgacttgtttacattatataaatgtcatcgtcaaaaattaaattttaaatcaacgatttatattcatttatcacaactatcgtatatctatctgttgaagttattgtatatttgtttttgaactatcgcagatatttgtcatgtggttggattttgttacatacttgcatcaccatggctacacacagaaattaccatggtaccgtggccaggtaaaaccataatcatttaattaactaccttcaattgttcattttcgttttaatttgattactcatgaattcaatttaacaggagtggagttatttaagaggtggccttacaaccgttgatcgtgactatggttggattaataacattcatcatgatattggcacacatgttgttcatcatcttttccctcaaatccctcattatcacttgattgaagcggtacttatttttttctcaaattttcaaaattttatctctttatgattaaattcgtatttaattatgttaacaacacctgcattgacagatatcgatattattattctaaacattttggtcatacacacctatgtattgatgtctattctggcaaatgctgatatcaaaaatcaaataacatagtagtgtgtgatcgagccaggattgcatatacacaattttttaaatatgttgtgtctggtatcagagtcatttaaaatttttgagaaatcttttataagaaattgaagttagttttagtattattgtaaaaaaaaatttaaatgaaaaatcaattgtggatgttgtttttactataggtcgtcttacacactctcaacgacggtcggtcttgtctgttaattaaactgacttaatttttttgttcatcgcagacaaaagcagcaaaaccagtatttggaaaatattaccgagagccagaaaaatcatccccaattccatttcatctaataaaatacctagcacaaagcatgaaacatgaccactttgttagtgacaccggtgaaattctgttttatcaaactgatcctgagttgaacaagaatcaccatgactaa

SEQ ID NO: 57 - DNA, 1338 bp, synthetic construct
  source 1..1338: mol_type = other DNA
atggctgtaacagcctctattcataatcacctatctcaaaaccaacaaacactaggccaagaaaccatggctgtctccaatggtggtgccgattttgaccctagtgctcctccaccgtttaagattgcagatattagagctgcaattccgaagcattgttgggtcaagagtacttggagatctctgagctatgttgttagagatgtcttagttgttgctgcattgattgttgctgcaattcggtttgatagctggtttttttggcctatttattggcctcttcaagggactatgttttgggcgatttttgttcttggacatgactggtaaaaatttatgttttttttttgtttttgttttgctcatgattatgccttttggttttgttcatgttttgaattgaatattgtgtttttgttttgaatagtggccatggaagcttctcagatagttattttgtgaatagcttggtgggccatgttttgcactcctcaattcttgttccataccatggatggttagtgaatttgttcttagaatctttttttattgttcattggtcaaatctgtaattgtgagtttgtcggatttacgatgatgacagattaaaacgtaatttttatcatttgattttgttaatttatctgttaattcgaacctgcactaaattcatgaagtgtactcaaatcaattttcttttgttcttatggttttttccaggagaattagccataggacacatcatcaaaaccatggaaatgttgagaaggatgagggtgccggtatgtgaattaaatgcttccatgtgttgtttttaattgatttaacatagtattcagaaccgtctcaaattttttaaggctttgaattgaataatcgagattcaattctgtcaaaattgatactataaattttatttaatcacggtttaattaagagattttttttttgaaatcttgtttaattactcagattttagacttatgcggcagtctgcttacacgccacaaatacgactttgattaaatgatgtaatctttaagaagttttttaaattaattatctgatttgttttttgtgtgtcattatcttagttgacagaaaaggtttacaagagtttagacagcatgacaaagtttgtgaggttcactgttcctttaccaatgtttgcatatccaatttatttggtgagtgattttagtttttaaattattagtactatttgaaaaaaaacataacagttgaagaatctgaattttaatattctgatgtgaaatttcacatttttcagtggagaagaagtcctggaaaatcaggttctcactttcatccagatag

SEQ ID NO: 58 - DNA, 1236 bp, synthetic construct
  source 1..1236: mol_type = other DNA
atggctgtaacagcctctattcataatcacctatctcaaaaccaacaaacactaggccaagaaaccatggctgtctccaatggtggtgccgattttgaccctagtgctcctccaccgtttaagattgcagatattagagctgcaattccgaagcattgttgggtcaagagtacttggagatctctgagctatgttgttagagatgtcttagttgttgctgcattgattgttgctgcaattcggtttgatagctggtttttttggcctatttattggcctcttcaagggactatgttttgggcgatttttgttcttggacatgactggtaaaaatttatgttttttttttgtttttgttttgctcatgattatgccttttggttttgttcatgttttgaattgaatattgtgtttttgttttgaatagtggccatggaagcttctcagatagttattttgtgaatagcttggtgggccatgttttgcactcctcaattcttgttccataccatggatggttagtgaatttgttcttagaatctttttttattgttcattggtcaaatctgtaattgtgagtttgtcggatttacgatgatgacagattaaaacgtaatttttatcatttgattttgttaatttatctgttaattcgaacctgcactaaattcatgaagtgtactcaaatcaattttcttttgttcttatggttttttccaggagaattagccataggacacatcatcaaattttttaaggctttgaattgaataatcgagattcaattctgtcaaaattgatactataaattttatttaatcacggtttaattaagagattttttttttgaaatcttgtttaattactcagattttagacttatgcggcagtctgcttacacgccacaaatacgactttgattaaatgatgtaatctttaagaagttttttaaattaattatctgatttgttttttgtgtgtcattatcttagttgacagaaaaggtttacaagagtttagacagcatgacaaagtttgtgaggttcactgttcctttaccaatgtttgcatatccaatttatttggtgagtgattttagtttttaaattattagtactatttgaaaaaaaacataacagttgaagaatctgaattttaatattctgatgtgaaatttcacatttttcagtggagaagaagtcctggaaaatcaggttctcactttcatccagatag

SEQ ID NO: 59 - DNA, 21 bp, synthetic construct
  source 1..21: mol_type = other DNA
gatgaagcttttggtcacttg

SEQ ID NO: 60 - DNA, 23 bp, synthetic construct
  source 1..23: mol_type = other DNA
taacgggcttatctggctaagta

SEQ ID NO: 61 - DNA, 24 bp, synthetic construct
  source 1..24: mol_type = other DNA
atactgcaaaaagtttcatattca

SEQ ID NO: 62 - DNA, 19 bp, synthetic construct
  source 1..19: mol_type = other DNA
gctatggtgaggtcgacaa

SEQ ID NO: 63 - DNA, 22 bp, synthetic construct
  source 1..22: mol_type = other DNA
agagacattttcattgtcactg

SEQ ID NO: 64 - DNA, 22 bp, synthetic construct
  source 1..22: mol_type = other DNA
tatatacaggaacccaggattc

SEQ ID NO: 65 - DNA, 22 bp, synthetic construct
  source 1..22: mol_type = other DNA
atctgttaattcgaacctgcac

SEQ ID NO: 66 - DNA, 23 bp, synthetic construct
  source 1..23: mol_type = other DNA
ttcagattcttcaactgttatgt

(19) United States
(12) Patent Application Publication
(10) Pub. No.: US 2023/0117816 A1
(43) Pub. Date: Apr. 20, 2023

(54) COMPOSITIONS AND METHODS COMPRISING PLANTS WITH REDUCED LIPOXYGENASE AND/OR DESATURASE ACTIVITIES

(71) Applicant: Benson Hill, Inc., St. Louis, MO (US)

(72) Inventors: Matthew Begemann, St. Louis, MO (US); Emma January, St. Louis, MO (US); Erin Zess, St. Louis, MO (US)

(73) Assignee: Benson Hill, Inc., St. Louis, MO (US)

(21) Appl. No.: 17/934,095

(22) Filed: Sep. 21, 2022

Related U.S. Application Data
(60) Provisional application No. 63/246,356, filed on Sep. 21, 2021.
(60) Provisional application No. 63/305,131, filed on Jan. 31, 2022.
(60) Provisional application No. 63/327,077, filed on Apr. 4, 2022.

(51) Int. Cl. C12N 15/82; C12N 15/11; C12N 9/22

(52) U.S. Cl. CPC C12N 15/8247; C12N 15/8213; C12N 15/11; C12N 9/22; C12N 15/8205; C12N 2310/20; C12N 2800/80

(57) ABSTRACT

Provided herein are plants and plant parts containing mutation in LOX and/or FAD genes. Also disclosed are plants and plant parts comprising decreased LOX and/or FAD activity. Furthermore, provided herein are plants and plant parts, and products (e.g., protein compositions, oil) produced therefrom having reduced level of hexanal and/or hexanol, hexanol, linolenic acid, increased levels of oleic acid. Such plants, plant parts, and plant products can have improved flavor characteristics relative to the control WT plants. Plant oil having a high oleic acid content and a low linoleic/linolenic acid content is also provided. Also disclosed herein are methods and compositions of producing such plants and plant parts.

### RELATED APPLICATIONS

This application claims priority to U.S. Provisional Application No. 63/246,356, filed on Sep. 21, 2021; U.S. Provisional Application No. 63/305,131, filed on Jan. 31, 2022; and U.S. Provisional Application No. 63/327,077, filed on Apr. 4, 2022. The content of each of the foregoing applications are incorporated herein by reference in its entirety.

### SEQUENCE LISTING

This application contains a Sequence Listing which is submitted herewith in electronically readable format. The Sequence Listing file was created on Sep. 20, 2022, is named “B88552_1300WO_SL.xml” and its size is 91.2 kb. The entire contents of the Sequence Listing in the sequence listing.xml file are incorporated by reference herein.

### FIELD OF THE INVENTION

This disclosure relates generally to plants and plant parts with improved flavor. The disclosure provides methods for improving flavor in plants by editing lipooxygenase and/or desaturase genes. Also provided herein are compositions for use in such methods.

### BACKGROUND OF THE INVENTION

Lipoxygenases (LOX; linoleate: oxygen reductase, E.C. 1.13.11.12) are non-heme iron-containing enzymes that catalyze the addition of molecular oxygen at either the C-9 or C-13 residue of fatty acids with a 1,4-pentadiene structure. Linoleic and linolenic acids are the most abundant fatty acids in the lipid fraction of plant membranes and are the major substrates for LOXs. Lipoxygenases catalyze the formation of Z, E-conjugated hydroperoxides (HPOs) from polyunsaturated fatty acids such as linoleic and linolenic acid (FIG. 1). LOXs are encoded by gene families (LOX) in most, if not all, plant species. The transcription of each gene member is under tight developmental control, and more than one member is often active at a specific developmental stage, accounting for the occurrence of multiple LOX isoforms. These exhibit distinct features for preference of substrate, kinetic parameters, and positional specificity of substrate oxygenation (Feussner and Wasternack, Annu Rev Plant Biol 53: 275-297 (2002)). In pea (Pisum sativum), LOX genes often exhibit tissue specificity and are developmentally regulated (Domoney et al., Planta 181: 35-43 (1990)).

Desaturases are enzymes, which can desaturate substrates in the fatty acid biosynthetic pathways to polyunsaturated fatty acids. A delta-12 desaturase (FAD2) catalyzes the insertion of a double bond into 18:1 (oleic acid), forming linoleic acid (18:2). A delta-15 desaturase (FAD3) catalyzes the insertion of a double bond into 18:2, forming linolenic acid (18:3) (FIG. 9). FAD2 is 1,164 bp long with an open reading frame coding for about 387 amino acids. The FAD2 gene has been classified into four types, namely, FAD2-1, FAD2-2, FAD2-3, and FAD2-4 on the basis of their site and pattern of expression. The four variations of the FAD2 gene show high sequence similarity, but show differences in their expression patterns and functions in fatty acid modification.

Linoleic and linolenic acids are polyunsaturated fatty acids (PUFAs) that are essential for health and nutrition, as these cannot be synthesized in humans and have to be supplied through diet. PUFAs make the edible oil more vulnerable to rancidity, decrease its flavor, and shorten its shelf life (Pandey et al., BMC Genetics 15:133. 10). The oxidative stability and nutritional value of the edible oil are dependent upon the fatty acid content of the oil, especially of oleic and linoleic acids (Cao et al., BMC Plant Biol). Oleic acid was found to have higher oxidative stability than linoleic acid, resulting in the extension of its shelf life (Ge et al., Genet. Mol. Res. 14 17482-17488). Hence, there can be many benefits of targeting biological molecules that modulate the levels of oleic and linolenic acid, in plants.

There is a great need to find economic and environmentally friendly ways to reduce fatty acid breakdown products which are a major source of off-flavors in commercial crops.

### SUMMARY OF THE INVENTION

The present disclosure provides methods and compositions for mutating lipooxygenase (LOX) and/or fatty acid desaturases (FAD) in plants or plant parts. In some aspects of the disclosure, modified plants, including plant parts and plant cells, having a loss of function mutation in LOX and/or FAD genes, are provided. Also provided are modified fruits, vegetables, protein composition, oil, and food/beverage products produced from such modified plants or plant parts, having reduced level of hexanal, hexanol, linolenic acid and/or increased levels of oleic acid.

Accordingly, in a first aspect, provided herein is a plant or plant part comprising decreased activity of a liopoxygenase. The activity of the liopoxygenase gene in the plant or plant part is decreased when compared to a control plant or plant part expressing wild-type activity of the corresponding lipoxygenase gene. The aforementioned plant comprises one or more insertions, substitutions, or deletions in one or more genes selected from the group consisting of LOX-2 and LOX-3. In some embodiments, the amount of hexanal and/or hexanol is reduced relative to a control plant. In some such embodiments, the amount of hexanal and/or hexanol is reduced by 50-90% when compared to a control plant (e.g., without mutation). In some embodiments, the amount of hexanal is reduced by at least 70% as compared to a control plant or plant part. In some embodiments, the amount of 1-hexanol is reduced by at least 80% as compared to a control plant or plant part. In some embodiments, the amount of linolenic acid is reduced by at least 50% when compared to a control plant or plant part. In some embodiments, the amount of linolenic acid plus linoleic acid is reduced by at least 4% as compared to a control plant or plant part. In some embodiments, the amount of oleic acid is increased relative to a control plant not comprising the one or more insertions, substitutions, or deletions in said one or more genes. In some embodiments, the amount of oleic acid is increased by at least 4% as compared to a control plant or plant part. In additional embodiments, the plant or plant part has improved flavor characteristics when compared to a control plant. In further embodiments, yield or total protein content of the plant or plant part is not significantly decreased, e.g., it is at least 80% (e.g., 80%, 85%, 90%, 95%, 99%, 100%, or more) as compared to a control plant or plant part.

In a second aspect, provided herein is a plant or plant part comprising decreased activity of fatty acid desaturase (FAD). The activity of the FAD gene in the plant or plant part is decreased when compared to a control plant or plant part expressing wild-type activity of the corresponding lipoxygenase gene. The aforementioned plant comprises one or more insertions, substitutions, or deletions in one or more genes selected from the group consisting of FAD2 and FAD3. In some embodiments of the aforementioned aspects, the amount of polyunsaturated lipid (e.g., hexanal, hexanol, 1-octen-3-ol, linolenic acid) is reduced relative to a control plant (e.g., not comprising the one or more insertions, substitutions, or deletions in the FAD genes, FAD2 and FAD3). In some such embodiments, the amount of polyunsaturated lipid (e.g., hexanal, hexanol, 1-octen-3-ol, linolenic acid) is reduced by 50-90% when compared to a control plant. In some embodiments, the amount of hexanal is reduced by at least 70% as compared to a control plant or plant part. In some embodiments, the amount of 1-hexanol is reduced by at least 80% as compared to a control plant or plant part. In some embodiments, the amount of linolenic acid is reduced by at least 50% when compared to a control plant or plant part. In some embodiments, the amount of linolenic acid plus linoleic acid is reduced by at least 4% as compared to a control plant or plant part. In some embodiments, the amount of oleic acid is increased relative to a control plant not comprising the one or more insertions, substitutions, or deletions in said one or more genes. In some embodiments, the amount of oleic acid is increased by at least 4% as compared to a control plant or plant part. In additional embodiments, the plant or plant part has improved flavor characteristics when compared to a control plant. In further embodiments, yield or total protein content of the plant or plant part is not significantly decreased, e.g., it is at least 80% (e.g., 80%, 85%, 90%, 95%, 99%, 100%, or more) as compared to a control plant or plant part.

In some embodiments, the one or more insertions, substitutions, or deletions reduces expression of a protein encoded by one or more genes selected from the group consisting of LOX-2, LOX-3, FAD2, and FAD3. The plant or plant part expresses reduced protein relative to a control plant, such as a control plant expressing corresponding protein encoded by wild-type LOX-2, wild-type LOX-3 wild-type FAD2 and wild-type FAD3 genes.

In specific embodiments, LOX-2 activity is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In specific embodiments, LOX-3 activity is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In specific embodiments, FAD2 activity is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In specific embodiments, FAD3 activity is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

In some embodiments of the above aspect, the one or more insertions, substitutions, or deletions reduces expression of the encoded LOX-2 protein relative to a control plant expressing a wild-type LOX-2 gene. In specific embodiments, LOX-2 protein expression is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% when compared to a control plant. In some embodiments of the above aspect, the one or more insertions, substitutions, or deletions reduces expression of the encoded LOX-3 protein relative to a control plant expressing a wild-type LOX-3 gene. In specific embodiments, LOX-3 protein expression is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% when compared to a control plant. In some embodiments of the above aspect, the one or more insertions, substitutions, or deletions reduces expression of the encoded FAD2 protein relative to a control plant expressing a wild-type FAD2 gene. In specific embodiments, FAD2 protein expression is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% when compared to a control plant. In some embodiments of the above aspect, the one or more insertions, substitutions, or deletions reduces expression of the encoded FAD3 protein relative to a control plant expressing a wild-type Fad3 gene. In specific embodiments, FAD3 protein expression is reduced by about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% when compared to a control plant.

In some embodiments, the one or more insertions, substitutions, or deletions is in a region that corresponds to a nucleotide region upstream of exon 7 of the LOX-2 gene (e.g., of Pisum sativum). In some such embodiments, the one or more insertions, substitutions, or deletions corresponds to a deletion in a nucleotide region corresponding to exon 4 of the LOX-2 gene (e.g., of Pisum sativum). In some embodiments, the one or more insertions, substitutions, or deletions, is at least partially in a region that corresponds to a nucleotide region of exon 4 of the LOX-3 gene (e.g., of Pisum sativum). In some embodiments, the one or more insertions, substitutions, or deletions is at least partially in regions that each correspond to (i) a nucleotide region upstream of exon 7 or a nucleotide region of exon 4 of the LOX-2 gene of Pisum sativum and (ii) exon 4 of the LOX-3 gene (e.g., of Pisum sativum). In some embodiments, the one or more insertions, substitutions, or deletions are at least partially in a region that corresponds to a nucleotide region of exon 1 of the FAD2B gene (e.g., of Pisum sativum). In some embodiments, the one or more insertions, substitutions, or deletions are in a region that corresponds to a nucleotide region of exon 2 of the FAD3C gene and/or exon 3 of the FAD3D gene (e.g., of Pisum sativum). In some embodiments, the one or more insertions, substitutions, or deletions are at least partially in a region that corresponds to a nucleotide region of exon 4 of the LOX-3 gene (e.g., of Pisum sativum) and of exon 2 of the FAD3C gene (e.g., of Pisum sativum). In certain embodiments, FAD2 gene is FAD2B. In certain other embodiments, FAD3 gene is FAD3C. In yet other embodiments, FAD3 gene is FAD3D.

In certain embodiments, the one or more insertions, substitutions, or deletions comprise a deletion of about 4-23 nucleotides, such as about 11 nucleotides. In particular embodiments, the deletion comprises nucleotides 1521 through 1531 of SEQ ID NO: 10. In certain embodiments, the deletion comprises about 4-23 nucleotides, such as about 8 nucleotides. In particular embodiments, the deletion comprises nucleotides 1523 through 1530 of SEQ ID NO: 10. In some instances, the deletion is an out-of-frame deletion. In other instances, the deletion is an in-frame, missense, or nonsense deletion.

In some embodiments, the LOX-2 protein comprises: (a) an amino acid sequence having at least 90% sequence identity to the amino acid sequence set forth in SEQ ID NO: 7, wherein said LOX-2 protein retains LOX-2 activity; or (b) the amino acid sequence set forth in SEQ ID NO: 7.

In some embodiments, the gene encoding a LOX-2 protein comprises: (a) a nucleic acid sequence having at least 90% sequence identity to the nucleic acid sequence set forth in SEQ ID NO: 10, wherein said nucleic acid sequence encodes a functional LOX-2 protein; or (b) the nucleic acid sequence set forth in SEQ ID NO: 10.

In some embodiments, the LOX-3 protein comprises: (a) an amino acid sequence having at least 90% sequence identity to the amino acid sequence set forth in SEQ ID NO: 25, wherein said LOX-3 protein retains LOX-3 activity; or (b) the amino acid sequence set forth in SEQ ID NO: 25.

In some embodiments, the gene encoding a LOX-3 protein comprises: (a) a nucleic acid sequence having at least 90% sequence identity to the nucleic acid sequence set forth in SEQ ID NO: 27, wherein said nucleic acid sequence encodes a functional LOX-3 protein; or (b) the nucleic acid sequence set forth in SEQ ID NO: 27.

In some embodiments, the FAD2B protein comprises: (a) an amino acid sequence having at least 90% sequence identity to the amino acid sequence set forth in SEQ ID NO: 33, wherein said FAD2B protein retains FAD2B activity; or (b) the amino acid sequence set forth in SEQ ID NO: 33. In some embodiments, the FAD3C protein comprises: (a) an amino acid sequence having at least 90% sequence identity to the amino acid sequence set forth in SEQ ID NO: 43, wherein said FAD3C protein retains FAD3C activity; or (b) the amino acid sequence set forth in SEQ ID NO: 43. In some embodiments, the FAD3D protein comprises: (a) an amino acid sequence having at least 90% sequence identity to the amino acid sequence set forth in SEQ ID NO: 53, wherein said FAD3D protein retains FAD3D activity; or (b) the amino acid sequence set forth in SEQ ID NO: 53.

In some embodiments, the gene encoding a FAD2B protein comprises: (a) a nucleic acid sequence having at least 90% sequence identity to the nucleic acid sequence set forth in SEQ ID NO: 36, wherein said nucleic acid sequence encodes a functional FAD2B protein; or (b) the nucleic acid sequence set forth in SEQ ID NO: 36. In some embodiments, the gene encoding a FAD3C protein comprises: (a) a nucleic acid sequence having at least 90% sequence identity to the nucleic acid sequence set forth in SEQ ID NO: 46, wherein said nucleic acid sequence encodes a functional FAD3C protein; or (b) the nucleic acid sequence set forth in SEQ ID NO: 46. In some embodiments, the gene encoding a FAD3D protein comprises: (a) a nucleic acid sequence having at least 90% sequence identity to the nucleic acid sequence set forth in SEQ ID NO: 56, wherein said nucleic acid sequence encodes a functional FAD3D protein; or (b) the nucleic acid sequence set forth in SEQ ID NO: 56.

In some embodiments, the plant comprises 2-4 genes encoding a LOX-2 protein. In some such embodiments, the plant comprises at least 2 genes encoding a LOX-2 protein, wherein said genes have less than 99% sequence identity.

In some embodiments, the plant is selected from leguminous plants (e.g., pea (Pisum sativum), bean (Phaseolus spp.), soybean (Glycine max), chickpea (Cicer arietinum), peanut (Arachis hypogaea), lentils (Lens culinaris, Lens esculenta), fava bean (Vicia faba), mung bean (Vigna radiata), lupins (Lupinus spp.), mesquite (Prosopis spp.), carob (Ceratonia siliqua), tamarind (Tamarindus indica), alfalfa (Medicago sativa), and clover (Trifolium spp.)), oilseed plants (e.g., canola (Brassica napus), cotton (Gossypium sp.), camelina (Camelina sativa) and sunflower (Hehanthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa).

In another aspect, the present disclosure provides a protein composition isolated from the plant or plant part described hereinabove, wherein the protein composition has decreased amount of hexanal and/or hexanol when compared to protein composition isolated from a control plant.

In another aspect, the present disclosure provides a method of decreasing the amount of hexanal, hexanol, and/or linolenic acid in a plant or plant part when compared to a control plant or plant part. The method comprises decreasing the activity of one or more genes in the plant selected from the group consisting of LOX-2, LOX-3, FAD2, and FAD3, wherein said control plant or plant part expresses one or more wild-type genes selected from the group consisting of LOX-2, LOX-3, FAD2, and FAD3, and wherein decreasing the activity of one or more genes comprises introducing into said plant or plant part one or more insertions, substitutions, or deletions in one or more genes selected from the group consisting of LOX-2, LOX-3, FAD2, and FAD3.

In some embodiments, decreasing the activity of one or more genes comprises introducing into said plant or plant part one or more insertions, substitutions, or deletions in one or more genes selected from the group consisting of LOX-2, LOX-3, FAD2, and FAD3. In some such embodiments, the one or more insertions, substitutions, or deletions reduces the expression of the encoded proteins LOX-2, LOX-3, FAD2, and/or FAD3, relative to a control plant. In some embodiments, the method further comprising increasing the amount of oleic acid in the plant or plant part when compared to the control plant or plant part.

In some embodiments, decreasing the production of LOX-2 or LOX-2 activity, comprises introducing into the plant or plant part one or more insertions, substitutions, or deletions in a gene encoding a LOX-2 protein. In some such embodiments, the one or more insertions, substitutions, or deletions reduces the expression of the encoded LOX-2 protein relative to a control plant.

In certain embodiments, the one or more insertions, substitutions, or deletions corresponds to a deletion in a nucleotide region corresponding to exon 4 of the LOX-2 gene of Pisum sativum. In some embodiments, the one or more insertions, substitutions, or deletions is at least partially in a region that corresponds to a nucleotide region of exon 4 of the LOX-3 gene of Pisum sativum. In some embodiments, the one or more insertions, substitutions, or deletions, or part thereof is at least partially in regions that each correspond to (i) a nucleotide region upstream of exon 7 or a nucleotide region of exon 4 of the LOX-2 gene of Pisum sativum and (ii) exon 4 of the LOX-3 gene of Pisum sativum. In some embodiments, the one or more insertions, substitutions, or deletions is at least partially in a region that corresponds to a nucleotide region of exon 2 of the FAD3C gene of Pisum sativum. In some embodiments, the one or more insertions, substitutions, or deletions is at least partially in a region that corresponds to a nucleotide region of exon 3 of the FAD3D gene of Pisum sativum. In some embodiments, the one or more insertions, substitutions, or deletions is at least partially in a region that corresponds to a nucleotide region of exon 4 of the LOX-3 gene of Pisum sativum and of exon 2 of the FAD3C gene of Pisum sativum. In certain embodiments, FAD2 gene is FAD2B. In certain other embodiments, FAD3 gene is FAD3C. In yet other embodiments, FAD3 gene is FAD3D.

In some such embodiments, the method comprises introducing into said plant or plant part a deletion comprising about 2-107 nucleotides, such as about 2, 5, 8, 11, 28, 49, or 107 nucleotides. In particular embodiments, the deletion comprises nucleotides 1521 through 1531 of SEQ ID NO: 10. In particular embodiments, the deletion comprises nucleotides 1523 through 1530 of SEQ ID NO: 10. In other embodiments, said plant or plant part comprises SEQ ID NO: 5 or 6. In particular embodiments, the deletion comprises nucleotides 1129 through 1156 of SEQ ID NO: 27 or said plant or plant part comprises SEQ ID NO: 24. In specific embodiments, the deletion comprises nucleotides 59 through 66 of SEQ ID NO: 36 or nucleotides 60 through 61 of SEQ ID NO: 36. In other embodiments, said plant or plant part comprises SEQ ID NO: 31 or 32. In particular embodiments, the deletion comprises nucleotides 457 through 464 of SEQ ID NO: 46 or nucleotides 416 to 464 of SEQ ID NO: 46. In other embodiments, said plant or plant part comprises SEQ ID NO: 41 or 42. In specific embodiments, the deletion comprises nucleotides 775 through 779 of SEQ ID NO: 56 or nucleotides 745 through 851 of SEQ ID NO: 56. In other embodiments, said plant or plant part comprises SEQ ID NO: 51 or 52.

In some instances, the deletion is an out-of-frame deletion. In other instances, the deletion is an in-frame, missense, or nonsense deletion.

In some embodiments of the method described above, decreasing the activity of one or more genes selected from the group consisting of LOX-2, LOX-3, FAD2, and FAD3 comprises introducing into said plant a nucleic acid construct encoding at least one site-directed nuclease that is specific for a target site in the genome of the plant, wherein, upon expression, the nuclease cleaves the plant genome at the target site, resulting in one or more insertions, substitutions, or deletions and the activity of one or more genes selected from the group consisting of LOX-2, LOX-3, FAD2, and FAD3 is decreased. In such embodiments, the nuclease cleaves DNA in the plant to alter the plant's gene expression.

In some embodiments, the one or more insertions, substitutions, or deletions in a gene encoding a LOX protein or a FAD protein, are introduced following cleavage of one or more genes selected from the group consisting of LOX-2, LOX-3, FAD2, and FAD3 by an endonuclease that is part of a Type II or Type V CRISPR system. In such embodiments, the endonuclease that is part of a Type II or Type V CRISPR system is a Cas9 nuclease, a Cpf1(Cas12a) nuclease, or a Cms1 nuclease. In a specific embodiment, the endonuclease is a Cas12a endonuclease. In another specific embodiment, the endonuclease is a Cas12a endonuclease orthologue. In some specific embodiments, the endonuclease comprises an amino acid sequence having at least 80% sequence identity to the amino acid sequence set forth in SEQ ID NO: 16.

In some embodiments, the method described herein above further comprises at least one guide RNA (gRNA) operatively arranged with the endonuclease for genomic editing of one or more genes selected from the group consisting of LOX-2, LOX-3, FAD2, and FAD3, binding the gRNA. In some embodiments, the gRNA targets a nucleotide region corresponding to exon 4 of the LOX-2 gene, exon 4 of the LOX-3 gene, exon 1 of the FAD2B gene, exon 2 of the FAD3C gene, or exon 3 of the FAD3D gene. In some embodiments, the gRNA comprises a polynucleotide sequence encoded by a nucleic acid sequence comprising any one of SEQ ID NOs: 4, 23, 30, 40, and 50. In some embodiments of the above aspect, decreasing the production or expression of a protein comprises introduction of interfering RNA, and/or modification of regulatory elements of one or more genes selected from the group consisting of LOX-2, LOX-3, FAD2, and FAD3.

In some embodiments of the aforementioned methods, the amount of hexanal, hexanol, 1-octen-3-ol, and/or linolenic acid is reduced by 50-90% when compared to a control plant. In some embodiments, the amount of hexanal is reduced by at least 70% in the plant or plant part as compared to a control plant or plant part. In some embodiments, the amount of 1-hexanol is reduced by at least 80% in the plant or plant part as compared to a control plant or plant part. In some embodiments, the amount of linolenic acid is reduced by at least 50% in the plant or plant part when compared to a control plant or plant part. In some embodiments, the amount of linolenic acid plus linoleic acid is reduced by at least 4% in the plant or plant part as compared to a control plant or plant part. In some embodiments, the amount of oleic acid is increased in the plant or plant part relative to a control plant not comprising the one or more insertions, substitutions, or deletions in said one or more genes. In some embodiments, the amount of oleic acid is increased by at least 4% in the plant or plant part as compared to a control plant or plant part. In some embodiments of the method, the plant or plant part has improved flavor characteristics when compared to a control plant or plant part. In further embodiments, yield or total protein content of the plant or plant part is not significantly decreased, e.g., it is at least 80% (e.g., 80%, 85%, 90%, 95%, 99%, 100%, or more than 100%) as compared to a control plant or plant part.

In some embodiments, the plant is selected from leguminous plants (e.g., pea (Pisum sativum), bean (Phaseolus spp.), soybean (Glycine max), chickpea (Cicer arietinum), peanut (Arachis hypogaea), lentils (Lens culinaris, Lens esculenta), fava bean (Vicia faba), mung bean (Vigna radiata), lupins (Lupinus spp.), mesquite (Prosopis spp.), carob (Ceratonia siliqua), tamarind (Tamarindus indica), alfalfa (Medicago sativa), and clover (Trifolium spp.)), oilseed plants (e.g., canola (Brassica napus), cotton (Gossypium sp.), camelina (Camelina sativa) and sunflower (Hehanthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa).

In some embodiments, the method described hereinabove further comprises isolating, extracting, and/or preparing a protein composition (e.g., soy protein composition, pea protein composition, soy/pea protein concentrate (SPC/PPC), soy/pea protein isolate (SPI/PPC), soy/pea flour, white flake, texturized vegetable protein (TVP), textured soy protein (TSP)) from the plant or plant part, such as from seed. In some such embodiments, the protein composition has decreased hexanal, hexanol, and/or linolenic acid amounts when compared to a protein composition isolated or extracted from a control plant. In additional embodiments, the protein composition has improved flavor characteristics when compared to protein composition isolated from a control plant.

In another aspect, the present disclosure provides a protein composition isolated or extracted by the methods described hereinabove. In some embodiments, the protein composition is isolated or extracted from a pea plant.

In one aspect, the present disclosure provides oil produced from the plant or plant part provided herein, or from a plant or plant part produced by the method provided herein, wherein the oil comprises: high oleic acid content; low linoleic acid content; low linolenic acid content; high oleic acid and low linoleic acid content; high oleic acid and low linolenic acid content; low linoleic acid and low linolenic acid content; or high oleic acid, low linoleic acid, and low linolenic acid content, relative to oil produced from a control plant or plant part. In some embodiments, the oil comprises high monounsaturated fatty acid to polyunsaturated fatty acid composition relative to oil produced from a control plant or plant part. In some embodiments, the oil comprises at least about 4% increase in oleic acid content and/or at least about 4% decrease in linoleic plus linolenic acid content relative to oil produced from a control plant or plant part. In some embodiments, the oil comprises a linolenic acid content of about 4% to about 10%. In some embodiments, the oil further comprises an oleic acid content of about 30% to about 40%, and a linoleic plus linolenic acid content of about 45% to 55%.

In one aspect, the present disclosure provides plant oil comprising a linolenic acid content of about 4% to about 10%. In some embodiments, the plant oil further comprises an oleic acid content of about 30% to about 40%, and a linoleic plus linolenic acid content of about 45% to 55%.

In some embodiments, the oil, plant oil, or protein composition provided herein comprises one or more nucleic acid molecules each comprising a nucleic acid sequence of a mutated LOX-2, LOX-3, FAD2, or FAD3 gene or fragment thereof. In some embodiments, said one or more nucleic acid molecules in the oil or protein composition each comprise a nucleic acid sequence of: (i) a mutated LOX-2 gene or a fragment thereof comprising a deletion of nucleotides 1521 through 1531 of SEQ ID NO: 10; (ii) a mutated LOX-2 gene or a fragment thereof comprising a deletion of nucleotides 1523 through 1530 of SEQ ID NO: 10; (iii) a mutated LOX-3 gene or a fragment thereof comprising a deletion of nucleotides 1129 through 1156 of SEQ ID NO: 27; (iv) a mutated FAD2B gene or a fragment thereof comprising a deletion of nucleotides 59 through 66 of SEQ ID NO: 36; (v) a mutated FAD2B gene or a fragment thereof comprising a deletion of nucleotides 60 through 61 of SEQ ID NO: 36; (vi) a mutated FAD3C gene or a fragment thereof comprising a deletion of nucleotides 457 through 464 of SEQ ID NO: 46; (vii) a mutated FAD3C gene or a fragment thereof comprising a deletion of nucleotides 416 through 464 of SEQ ID NO: 46; (viii) a mutated FAD3D gene or a fragment thereof comprising a deletion of nucleotides 775 through 779 of SEQ ID NO: 56; or (ix) a mutated FAD3D gene or a fragment thereof comprising a deletion of nucleotides 745 through 851 of SEQ ID NO: 56.

In another aspect, the present disclosure provides a food or beverage product comprising the protein composition or oil described herein.

### BRIEF DESCRIPTION OF THE DRAWINGS

FIG. 1 is a schematic representation of lipoxygenase-mediated catalysis of polyunsaturated fatty acids, such as linoleic and linolenic acid, that results in the formation of Z, E-conjugated hydroperoxides (HPOs).

FIG. 2 is a schematic representation of Pea LOX-2 gene structure. Eight exons (exon I-VIII) are shown as solid bars; 7 introns are shown as dashed lines.

FIG. 3 is a schematic representation of the map of Construct A.

FIG. 4 is a graph showing percent editing efficiency for LOX-2 guide RNA at three off-target sites (OT1, OT2, and OT6) and at the LOX-2 exon 4 site in the edited (Plant K) and the wild-type (WT) plants.

FIG. 5 provides western blot analysis of LOX-2 protein in GE LOX-2 seed. Total protein from pea plant seeds were extracted and electrophoresed on an 8% polyacrylamide gel, western blotted, and probed with the Lox2-L3 antibody. SM=Molecular Weight markers; GE-lox2, Plant E seed; GE-Null, Plant C seed; Amigo, WT seed; Lox2, Lox-2 reference protein; Lox3, Lox-3 reference protein.

FIG. 6 provides western blot analysis of LOX-2 protein in GE LOX-2 seed. Total protein from pea plant seeds were extracted and electrophoresed on an 8% polyacrylamide gel, western blotted, and probed with the Lox2-L1 antibody. SM=Molecular Weight markers; GE-lox2, Plant E seed; GE-Null, Plant C seed; Amigo, WT seed; Lox2, Lox2 reference protein; Lox3, Lox3 reference protein.

FIG. 7 is a graph showing LOX-2 enzyme activity in WT, Plant C , and Plant E seed samples.

FIG. 8 shows a phylogenetic tree of soybean and pea lipoxygenases. This phylogenetic tree shows that pea lipoxygenases 1 and 2 are closely related, while pea lipoxygenase 3 is a more distant relative.

FIG. 9 is a schematic representation of the fatty acid biosynthetic pathway in higher plants including in Pisum sativum plant.

FIG. 10 shows a phylogenetic tree of soybean and pea FADs. This phylogenetic tree shows that pea FAD2-1s and FAD2-2s are distant relative.

FIGS. 11A-B show differential expression of FAD2 in different plant tissue. FIG. 11A shows differential expression of FAD2A in different plant tissue. FIG. 11B shows differential expression of FAD2B in different plant tissue.

FIG. 12 shows differential expression of FAD2A and FAD2B in different plant tissue. The gene expression data on the right shows significantly detailed information of the FAD2A and FAD2B expression compared to the corresponding publicly available expression information shown on the left.

FIG. 13 shows a phylogenetic tree of soybean and pea FADs. This phylogenetic tree shows that pea FAD3s, FAD7s and FAD8s are distant relative.

FIGS. 14A-B shows differential expression of FAD3s in different plant tissue. FIG. 14A shows differential expression of FAD3C in different plant tissue. FIG. 14B shows differential expression of FAD3D in different plant tissue.

FIG. 15 shows differential expression of FAD3C and FAD3D in different plant tissue. The gene expression data on the right shows significantly detailed information of the FAD3C and FAD3D expression compared to the corresponding publicly available expression information shown on the left.

FIGS. 16A-B shows differential expression of FAD3C and FAD3D in different plant tissue. Embryonic axes of mature seeds of yellow pea variety Amigo was transformed with LOX-2 variant constructs shown in FIG. 3 using Agrobacterium transformation and a LOX2 edited yellow pea plant was successfully generated (FIG. 16A). Embryonic axes of mature seeds of yellow pea variety Maxum was transformed with LOX-2 variant constructs shown in FIG.16B using Agrobacterium transformation and an LOX2 edited yellow pea plant was successfully generated (FIG. 16B).

FIG. 17 shows the data confirming the validated editing of FAB2B gene in a pea plant. The Cpf1 nuclease variant was used to edit FAD2B gene the Amigo variety of pea plant. The guide sites and the guide site edit validation data shows the edit efficiency of different FAD2B alleles.

FIG. 18 shows examples of three highly edited events for FAD2B identified in the T0 generation of the Amigo variety of pea plants. The edits were confirmed by NGS sequencing.

FIG. 19 shows a plot demonstrating a significant reduction of off-flavor compounds such as hexanal and hexanol in LOX-3 and FAD3C knockout yellow pea line. The relative peak area was used to compare the intensity of volatile production in the edited with edited LOX-3 and FAD3C gene plant and the non-edited plant.

### DETAILED DESCRIPTION OF THE INVENTION

### 1.1 References and Definitions

The present disclosure now will be described more fully hereinafter. The disclosure may be embodied in many different forms and should not be construed as limited to the aspects set forth herein; rather, these aspects are provided so that this disclosure will satisfy applicable legal requirements.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

“Fatty acid” refers to free fatty acids and fatty acyl groups. The terms “fatty acid desaturase” and “FAD” are used interchangeably herein and refer to membrane bound microsomal oleoyl- and linoleoyl-phosphatidylcholine desaturases that convert oleic acid to linoleic acid and linoleic acid to linolenic acid, respectively, in reactions that reduce molecular oxygen to water and require the presence of NADH “FAD2” refers to a gene or encoded protein capable of catalyzing the insertion of a double bond into a fatty acyl moiety at the twelfth position counted from the carboxyl terminus. FAD2 proteins are also referred to as “412 desaturase” or “omega-6 desaturase”. refers to a gene or encoded protein capable of catalyzing the insertion of a double bond into a fatty acyl moiety at the twelfth position counted from the carboxyl terminus. FAD2 proteins are also referred to as “Δ12 desaturase” or “omega-6 desaturase”.

The term “FAD2-1” is used to refer to a FAD2 gene or protein that is naturally expressed in a specific manner in seed tissue, and the term “FAD2-2” is used to refer a FAD2 gene or protein that is (a) a different gene from a FAD2-1 gene or protein and (b) is naturally expressed in multiple tissues, including the seed. Representative FAD2 sequences include, without limitation, those set forth in U.S. patent application Ser. No. 10/176,149 filed on Jun. 21, 2002, and in SEQ ID NOs: 33 and 36, which are incorporated herein in their entirety.

A “FAD3”, “Δ15 desaturase” or “omega-3 desaturase” gene encodes an enzyme (FAD3) capable of catalyzing the insertion of a double bond into a fatty acyl moiety at the fifteenth position counted from the carboxyl terminus. The terms “FAD3-1”, “FAD3A”, “FAD3B”, “FAD3C”, and “FAD3D” are used to refer to FAD3 gene family members that are naturally expressed in multiple tissues, including the seed. Representative FAD3 sequences include, without limitation, those set forth in U.S. patent application Ser. No. 10/176,149 filed on Jun. 21, 2002, and in SEQ ID NOs: 43, 46, 53, and 56, which are incorporated herein in their entirety.

The terms “lipoxygenase” and “LOX” are used interchangeably herein. These terms refer to any member of a group of enzymes (e.g., LOX2, LOX3) that catalyze the hydroperoxidation of polyunsaturated fatty acids in the first step of fatty acid metabolite synthesis. In the higher plant lipoxygenase pathway, linoleic acid and linolenic acid are oxygenated by the action of lipoxygenase (LOX) to produce hydroperoxide fatty acids.

As used herein, the term “gene” refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, a “mutation” is any change in a nucleic acid sequence. In particular, as used herein, a “mutation” may refer to any change in the nucleic acid sequence of LOX (e.g., LOX-2, LOX-3) and/or FAD (e.g., FAD2B, FAD3C) genes. Non-limiting examples of mutation comprise insertions, deletions, duplications, substitutions, inversions, and translocations of any nucleic acid sequence, regardless of how the mutation is brought about and regardless of how or whether the mutation alters the functions or interactions of the nucleic acid. For example, a mutation may produce, without limitation, altered enzymatic activity of a ribozyme, altered base pairing between nucleic acids (e.g., RNA interference interactions, DNA-RNA binding, etc.), altered mRNA folding stability, and/or how a nucleic acid interacts with polypeptides (e.g., DNA-transcription factor interactions, RNA-ribosome interactions, gRNA-endonuclease reactions, etc.). A mutation might result in the production of proteins with altered amino acid sequences (e.g., missense mutations, nonsense mutations, frameshift mutations, etc.) and/or the production of proteins with the same amino acid sequence (e.g., silent mutations). Certain synonymous mutations may create no observed change in the plant while others that encode for an identical protein sequence nevertheless result in an altered plant phenotype (e.g., due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements, such as tRNAs, ribozymes, ribosome components, and microRNAs.

Accordingly, “plant with a mutation” or “plant part with a mutation” or “plant cell with a mutation” or “plant genome with a mutation” refers to a plant or plant part or plant cell or plant genome that contains a mutation described in the present disclosure, such as a mutated LOX (e.g., LOX-2, LOX-3) and/or FAD (e.g., FAD2B, FAD3C) genes. For example, as used herein, a plant, plant part or plant cell with a mutation may refer to a plant, plant part or plant cell in which, or in an ancestor of which, a LOX (e.g., LOX-2, LOX-3) and/or a FAD (e.g., FAD2B, FAD3C) gene has been deliberately mutated such that the plant, plant part or plant cell expresses a truncated LOX (e.g., LOX-2, LOX-3) and/or a FAD (e.g., FAD2B, FAD3C) proteins or otherwise modified LOX (e.g., LOX-2, LOX-3) and/or a FAD (e.g., FAD2B, FAD3C) proteins. The truncated or modified LOX (e.g., LOX-2, LOX-3) and/or a FAD (e.g., FAD2B, FAD3C) proteins can have reduced function or loss-of-function. The term “plant part” as used herein includes, without limitation, seed, endosperm, ovule, pollen, roots, tubers, stems, leaves, stalks, fruit, berries, nuts, bark, pods, seeds and flowers. In a particular embodiment of the present invention, the plant part is a seed (e.g., pea).

As used herein, the term “LOX function” or “LOX activity” refers to the enzyme activity of LOX (e.g., LOX-2, LOX-3) enzymes described herein. For example, “LOX-2 function” or “LOX-2 activity” can refer to the ability of LOX-2 to catalyze co-oxidation of a substrate, such as a polyunsaturated fatty acid. Enzyme activity or function of the lipoxygenases can be determined by using a polyunsaturated fatty acid, such as linolenic acid, as a substrate. Details of such procedure has been outlined in the Examples section of the present disclosure. In some instances, “LOX-2 function” or “LOX-2 activity” can refer to role of LOX-2 in oxidation of polyunsaturated fatty acids to produce 6-carbon aldehydes, such as hexanal and/or hexanol. “LOX function” or “LOX activity” may also refer to the ability of LOX to catalyze co-oxidation of pigments and proteins that influences post-harvest quality of fruits and vegetables by destroying antioxidants, bleaching colors, and generating aromas from pigment breakdown. Thus, “reduced function” or “loss-of-function” of LOX or LOX-2 or LOX-3 can refer to reduced ability or loss of ability of LOX or LOX-2 or LOX-3, respectively, to form hexanal and/or hexanol in plants or plant parts. A “loss-of-function mutation” is a mutation in the coding sequence of a gene, which causes the function of the gene product, usually a protein, to be either reduced or completely absent. A loss-of-function mutation can, for instance, be caused by the truncation of the gene product because of a frameshift or nonsense mutation. A phenotype associated with an allele with a loss of function mutation can be either recessive or dominant. For example, a plant or plant part that contains a mutated LOX-2 and/or LOX-3 gene can express a truncated LOX-2 and/or LOX-3 protein, or otherwise modified LOX-2 and/or LOX-3 protein, with reduced function or loss-of-function, which may reduce the level of hexanal and/or hexanol in such plant or plant part, as compared to a control plant or plant part that has a wild-type (WT) LOX-2 and/or LOX-3 gene. Alternatively, a plant or plant part that contains a mutated LOX-2 and/or LOX-3 gene can express reduced level of LOX-2 and/or LOX-3 protein, which results in overall reduction in LOX-2 and/or LOX-3 function, thus leading to reduced level of hexanal and/or hexanol in such plant or plant part, as compared to a control plant or plant part that has a WT LOX-2 and/or LOX-3 gene. Accordingly, the plant or plant part containing the mutated LOX-2 gene and expressing LOX-2 protein with reduced function or loss-of-function can have improved flavor, as compared to a control plant or plant part that has a WT LOX-2 gene. Similarly, the plant or plant part containing the mutated LOX-3 gene and expressing LOX-3 protein with reduced function or loss-of-function can have improved flavor, as compared to a control plant or plant part that has a WT LOX-3 gene.

As used herein, “FAD function” or “FAD activity” can refer to role of FAD (e.g., FAD2B) in the modulating the levels of long chain polyunsaturated fatty acid in the fatty acid metabolism pathway. Polyunsaturated fatty acids can be major precursors of the off-flavor compounds in yellow pea. The major polyunsaturated fatty acid in yellow pea, linoleic acid, is synthesized from the main product of the plastidial fatty acid biosynthesis, oleic acid, by membrane bound FAD2. In some instances, “FAD2 function” or “FAD2 activity” can refer to role of FAD (e.g., FAD2B) in the introduction of a second double bond into oleic acid to form a linoleic acid, a polyunsaturated fatty acid, in the fatty acid metabolism pathway. “FAD3 function” or “FAD3 activity” may also refer to the ability of FAD3 (e.g., FAD3D) to introduce a third double bond into linoleic acid (18:2) to form linolenic acid (18:3). Thus, “reduced function” or “loss-of-function” of FAD, FAD2, or FAD3 can refer to reduced ability or loss of ability of FAD, FAD2, or FAD3 to modulate or catalyze the formation of poly unsaturated fatty acids, particularly to form linolenic acid in plants or plant parts. For example, a plant or plant part that contains a mutated FAD2 gene can express a truncated FAD2 protein, or otherwise modified FAD2 protein, with reduced function or loss-of-function, may have an accumulation of oleic acid and corresponding decrease in polyunsaturated fatty acid content, especially linolenic acid. Reduction of expression of FAD3 in combination with reduction of FAD2 can lead to an even greater accumulation of oleic acid and corresponding decrease in polyunsaturated fatty acid content, especially linolenic acid. Accordingly, the plant or plant part containing the mutated FAD, FAD2, or FAD3 genes and expressing FAD, FAD2, or FAD3 proteins with reduced function or loss-of-function can have improved flavor, as compared to a control plant or plant part. In specific embodiments, control plants or plant parts can have a wild-type version of the FAD, FAD2, and/or FAD3 genes, or otherwise express a level analogous to wild-type level of FAD, FAD2, and/or FAD3 proteins or have a wild-type level of FAD, FAD2, and/or FAD3 activity. As used herein, a “control plant” or “control plant part” or “control cell” or “control seed” refers to a plant or plant part or plant cell or seed that does not contain a mutation described herein. For example, a “control plant” or “control plant part” or “control cell” or “control seed” may refer to a plant or plant part or cell or seed which does not contain a mutated LOX (e.g., LOX-2, LOX-3) and/or a FAD (e.g., FAD2B, FAD3C) genes, or if mutated, the LOX and/or FAD gene has at or near wild-type activity. In some embodiments, a control plant or control plant part or control cell or control seed refers to a plant or plant part or cell or seed that does not contain one or more modified polynucleotides of the present disclosure. For example, a “control plant”, “control plant part”, “control plant cell”, or “control seed” may refer to a plant, plant part, plant cell, or seed before a mutation of the present disclosure had been introduced into the plant, plant part, plant cell, or seed. Alternatively, a “control plant” or “control plant part” or “control cell” or “control seed” may refer to a plant or plant part or plant cell or seed, wherein a LOX (e.g., LOX-2, LOX-3) and/or a FAD (e.g., FAD2B, FAD3C) gene has not been mutated by the methods of the present disclosure. For example, a “control plant” or “control plant part” or “control cell” or “control seed” may refer to a plant or plant part or cell or seed that expresses an unmutated i.e., a WT LOX-2 gene or a LOX-2 gene with WT production and/or activity. Accordingly, such control plant or control plant part or control cell or control seed may express a fully-functional LOX-2 protein or amount of LOX-2 found in the corresponding WT plant or plant part. In certain instances, a control plant of the present disclosure is grown under the same environmental conditions (e.g., same or similar temperature, humidity, air quality, soil quality, water quality, and/or pH conditions) as a plant with mutation described herein. Similarly, a control protein or control protein composition can refer to a protein or protein composition that is isolated or extracted or derived from a control plant.

As used herein, “flavor” or “flavor characteristics” can refer to aroma or taste of plant, plant part (e.g. seed or pea pod), or protein composition obtained from a plant or plant part described herein. Aroma can relate to the ratios and intensities of volatile compounds, such as hexanol and/or hexanal in plant, plant part, or protein composition obtained from plant or plant part. In some embodiments, aroma can relate to the ratios and levels of degradation products of polyunsaturated fatty acids (PUFAs), such as linolenic acid in plant, plant part, or protein composition obtained from plant or plant part. PUFAs can be major precursors of the off-flavor compounds in yellow pea. Flavor characteristics can include aspects described as overall aroma, overall flavor impact, beany yellow pea, pyrazine, cereal grain, grassy green pea, nutty, cardboard, malty, salt, bitter, umami, astringent, and/or chalky.

Volatile compounds (e.g., hexanol, PUFAs, and their degradation products) that contribute to flavor characteristics of plant, plant part, or protein composition obtained from plant or plant part can be quantified by using Gas Chromatography—Mass Spectroscopy (GC-MS), a lab-based technique which helps to separate and identify compounds in their gaseous forms based on their masses. In certain instances, to correlate these instrumental measurements to consumer perception, two major methods of sensory evaluation are used: consumer testing and descriptive analysis. Consumer testing includes subjective data about the preferences of a large group of untrained tasters (usually more than 100 panelists), while descriptive analysis includes questionnaires for a panel of 8-12 trained tasters who are able to rate specific attributes related to flavor or aroma.

As used herein with respect to a parameter, the term “decreased” or “decreasing” or “decrease” or “reduced” or “reducing” or “reduce” or “lower” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, such as an established normal or reference level of the parameter, or an established standard control. Accordingly, the terms “decreased”, “reduced”, and the like encompass both a partial reduction and a complete reduction compared to a control.

For example, reduced hexanal and/or hexanol level in a plant or plant part may indicate an at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% decrease or negative change in a level of hexanal and/or hexanol in a plant or plant part, as compared to that in a control plant or plant part. Similarly, for example, reduced linolenic acid level in a plant or plant part may indicate an at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% decrease or negative change in a level of linolenic acid in a plant or plant part, as compared to that in a control plant or plant part.

As used herein with respect to a parameter, the term “increased” or “increasing” or “increase” or “higher” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, 120%, 150%, 200%, 300%, 400%, 500%, or more) positive change in the parameter from a comparison control, e.g., an established normal or reference level of the parameter, or an established standard control. Accordingly, the terms “increased”, “higher”, and the like encompass both a partial increase and a significant increase compared to a control.

When reference is made to particular nucleic acid sequences, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein, the term “expression” or “expressing” refers to the transcription and/or translation of a particular nucleotide sequence driven by a promoter. The terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild-type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the phrases “decreased activity” or “suppression of activity” are used interchangeably and refer to the reduction of the level of enzyme activity detectable in a plant with one or more insertions, substitutions, or deletions in one or more lipooxygenase and/or fatty acid desaturase genes when compared to the level of enzyme activity detectable in a plant with the native enzymes. The level of enzyme activity in a plant with the native enzyme is referred to herein as “wild-type” activity. The term “decrease” or “suppression”, in this context, includes lower, reduce, decline, decrease, inhibit, eliminate, and prevent. This reduction may be due to the decrease in translation of the native mRNA into an active enzyme. It may also be due to the transcription of the native DNA into decreased amounts of mRNA and/or to rapid degradation of the native mRNA. The term “native enzyme” or “wildtype enzyme” refers to an enzyme or level of activity that is produced naturally in the desired cell.

As used herein, the term “endogenous” in reference to a gene or nucleotide sequence or protein is intended to mean a gene or nucleotide sequence or protein that is naturally comprised within or expressed by a cell. Endogenous genes can include genes that naturally occur in the cell of a plant, but that have been modified in the genome of the cell without insertion or replacement of a heterologous gene that is from another plant species or another location within the genome of the modified cell.

“Homolog” or “homologous sequence” may refer to both orthologous and paralogous sequences. Paralogous sequence relates to gene-duplications within the genome of a species. Orthologous sequence relates to homologous genes in different organisms due to ancestral relationship. Thus, orthologs are evolutionary counterparts derived from a single ancestral gene in the last common ancestor of given two species and therefore have great likelihood of having the same function. One option to identify homologs (e.g., orthologs) in monocot plant species is by performing a reciprocal BLAST search. This may be done by a first blast involving blasting the sequence-of-interest against any sequence database, such as the publicly available NCBI database which may be found at: ncbi.nlm.nih.gov. If orthologs in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An ortholog is identified when the sequence resulting in the highest score (best hit) in the first blast identifies in the second blast the query sequence (the original sequence-of-interest) as the best hit. Using the same rational a paralog (homolog to a gene in the same organism) is found. In case of large sequence families, the ClustalW program may be used [ebi.ac.uk/Tools/clustalw2/index.html], followed by a neighbor-joining tree (wikipedia.org/wiki/Neighbor-joining) which helps visualizing the clustering.

In some embodiments, the term “homolog” as used herein, refers to functional homologs of genes. A functional homolog is a gene encoding a polypeptide that has sequence similarity to a polypeptide encoded by a reference gene, and the polypeptide encoded by the homolog carries out one or more of the biochemical or physiological function(s) of the polypeptide encoded by the reference gene. In general, it is preferred that functional homologs and/or polypeptides encoded by functional homologs share at least some degree of sequence identity with the reference gene or polypeptide encoded by the reference gene.

Homology (e.g., percent homology, sequence identity+sequence similarity) can be determined using any homology comparison software computing a pairwise sequence alignment.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymo1.266:131-141; Altschul et al. (1997), Nucleic Acids Res. 25:3389-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Proc Natl Acad Sci 89:10915-9 (1992)). Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence.

According to some embodiments, the homology is a global homology, e.g., a homology over the entire amino acid or nucleic acid sequences of the invention and not over portions thereof.

The degree of homology or identity between two or more sequences can be determined using various known sequence comparison tools which are described in WO2014/102774.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

An expression construct can permit transcription of a particular polynucleotide sequence in a host cell (e.g., a bacterial cell or a plant cell). An expression cassette may be part of a plasmid, viral genome, or nucleic acid fragment. Typically, an expression cassette includes a polynucleotide to be transcribed, operably linked to a promoter. The term “operably linked” refers to the association of nucleic acid fragments on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “population” refers to a set comprising any number, including one, of individuals, objects, or data from which samples are taken for evaluation, e.g., estimating quantitative trait locus (QTL) effects and/or disease tolerance. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silico representations of plants. The member of a population need not be identical to the population members selected for use in subsequent cycles of analyses, nor does it need to be identical to those population members ultimately selected to obtain a final progeny of plants. Often, a plant population is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., fiber lint percent, fiber quality, seed protein content, seed carbohydrate content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, pulp, juice, kernels, ears, cobs, husks, stalks, roots, root tips, anthers, and the like. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product (e.g., extract) or byproduct that retains one or more polynucleotides disclosed herein.

A plant, or its environment, can be contacted with a wide variety of agriculture treatment agents. As used herein, an “agriculture treatment agent” or “treatment agent” or “agent” can refer to any exogenously provided compound that can be brought into contact with a plant tissue (e.g., a seed) or its environment that affects a plant's growth, development and/or performance, including agents that affect other organisms in the plant's environment when those effects subsequently alter a plant's performance, growth, and/or development (e.g., an insecticide that kills plant pathogens in the plant's environment, thereby improving the ability of the plant to tolerate the insect's presence). Agriculture treatment agents also include a broad range of chemicals and/or biological substances that are applied to seeds, in which case they are commonly referred to as seed treatments and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g., fungicides, insecticides, bactericides, etc.) hormones (abscisic acids, auxins, cytokinins, gibberellins, etc.) herbicides (e.g., glyphosate, atrazine, 2,4-D, dicamba, etc.), nutrients (e.g., a plant fertilizer), and/or a broad range of biological agents, for example, a seed treatment inoculant comprising a microbe that improves crop performance, e.g., by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g., microencapsulation). In certain embodiments, the agriculture treatment agent comprises a nanoparticle and/or the application of the agriculture treatment agent comprises the use of nanotechnology.

In certain embodiments, a user can combine the teachings herein with high-density molecular marker profiles spanning substantially the entire genome of a plant to estimate the value of selecting certain candidates in a breeding program in a process commonly known as genome selection.

As used herein, the term “fertilization” and/or “crossing” includes bringing the genomes of gametes together to form zygotes, and may also broadly include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e., egg vs. sperm), but rather the same type and techniques are used to effect the combination of their genomes into a regenerable cell. Other embodiments of fertilization and/or crossing include circumstances where the gametes originate from the same parent plant, i.e., a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

As used herein, the terms “nuclease” and “endonuclease” are used interchangeably to refer to naturally-occurring or engineered enzymes, which cleave a phosphodiester bond within a polynucleotide chain.

The term “transformation”, as used herein broadly refers to the process by which a plant host is genetically modified by the introduction of DNA by means of a bacterium (e.g., Agrobacteria) or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, and shoots. A plant tissue also refers to any clone of such a plant, seed, progeny, propagule whether generated sexually or asexually, and descendants of any of these, such as cuttings or seed.

The term “transformed” as used herein, refers to a plant cell or tissue into which a foreign DNA molecule, such as a construct (e.g., a vector comprising the CRISPR-Cas endonuclease system described herein), has been introduced. The introduced DNA molecule may be integrated into the genomic DNA of the recipient cell or tissue such that the introduced DNA molecule is transmitted to the subsequent progeny or it can be transiently expressed. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

### 2.1 Modification of Fatty Acid Biosynthetic Enzymes

In plants, lipoxygenases (LOX) and fatty acid desaturases (FAD) have been implicated in unpleasant flavor and odor formation, as many of the degradation products of the PUFAs, produced by the activity of LOX and/or FAD enzymes are off flavor compounds. Plants, particularly legumes (e.g., yellow pea, soybean) have lipoxygenase enzymes that break down fatty acids and produce hexanal from linoleic acid. These breakdown products have a taste profile of grassy, beany, and stale. LOX breaks down fatty acids through an oxidation reaction, specifically through the formation of 6 carbon aldehydes. In addition, the ability of LOX to catalyze co-oxidation of pigments and proteins influences post-harvest quality of fruits and vegetables by destroying antioxidants, bleaching colors, and generating aromas from pigment breakdown. Hence, there can be many benefits of targeting LOX in plants; for example, it can reduce unpleasant flavor and odor in fruits and vegetables, retain antioxidants therein, and/or preserve their natural color. Thus plants and plant products wherein the LOX (e.g., LOX-2, LOX-3) gene has been mutated can be useful for improving plant performance or improvement of flavor of commodity products produced from the plant, such as plant protein compositions.

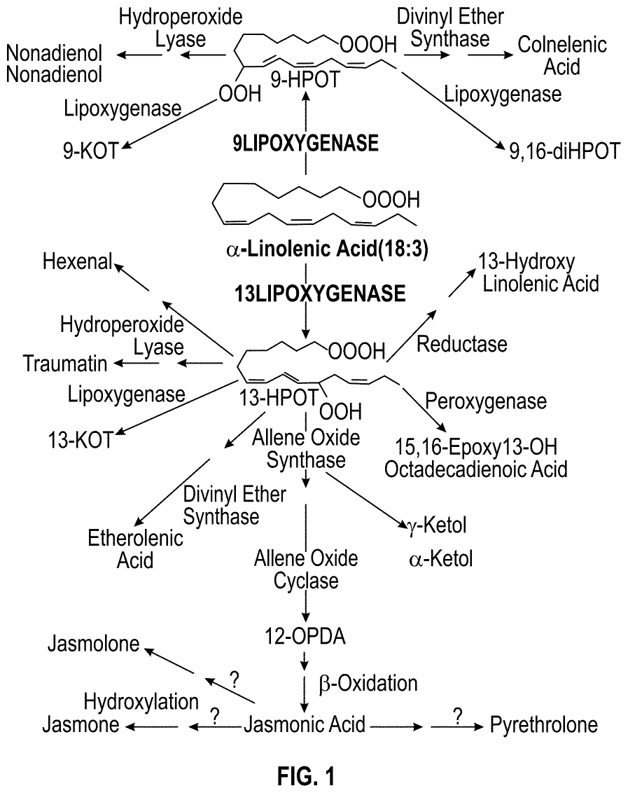

A phylogenetic tree of soybean lipoxygenases is shown in FIG. 1. This tree was assembled using the DNA Star suite and the amino acid sequences for known pea or soybean lipoxygenases. This tree clearly shows that pea and soybean LOX1 and LOX2 are closely related, while the pea and soybean LOX3 is a more distant relative.

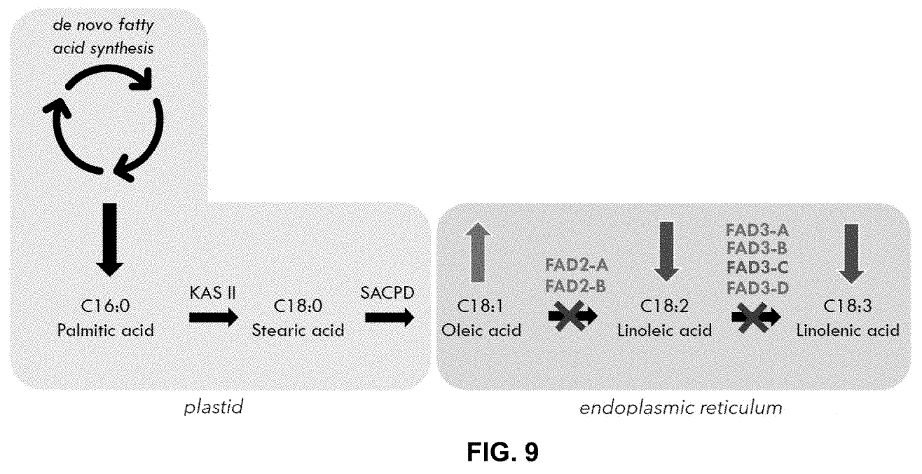

Higher plants synthesize fatty acids via the fatty acid synthetase (FAS) pathway, which is located in the plastids. β-ketoacyl-ACP synthases are important rate-limiting enzymes in the FAS of plant cells and exist in several versions. β-ketoacyl-ACP synthase I catalyzes chain elongation to palmitoyl-ACP (C16:0), whereas β-ketoacyl-ACP synthase II catalyzes chain elongation to stearoyl-ACP (C18:0) (FIG. 9). β-ketoacyl-ACP synthase IV is a variant of β-ketoacyl-ACP synthase II, and can also catalyze chain elongation to 18:0-ACP. In soybean, the major products of FAS are 16:0-ACP (palmitic acid) and 18:0-ACP (stearic acid). The desaturation of 18:0-ACP to form 18:1-ACP is catalyzed by a plastid-localized soluble delta-9 desaturase (also referred to as “stearoyl-ACP desaturase” or “SACPD”). See Voelker et al., 52 Annu. Rev. Plant Physiol. Plant Mol. Biol. 335-61 (2001). The products of the plastidial FAS and delta-9 desaturase, 16:0-ACP, 18:0-ACP, and 18:1-ACP, are hydrolyzed by specific thioesterases (FAT). Plant thioesterases can be classified into two gene families based on sequence homology and substrate preference. The first family, FATA, includes long chain acyl-ACP thioesterases having activity primarily on 18:1-ACP. Enzymes of the second family, FATB, commonly utilize 16:0-ACP (palmitoyl-ACP), 18:0-ACP (stearoyl-ACP), and 18:1-ACP (oleoyl-ACP). Such thioesterases have an important role in determining chain length during de novo fatty acid biosynthesis in plants, and thus these enzymes are useful in the provision of various modifications of fatty acyl compositions, particularly with respect to the relative proportions of various fatty acyl groups that are present in seed storage oils.

The products of the FATA and FATB reactions, the free fatty acids, leave the plastids and are converted to their respective acyl-CoA esters. Acyl-CoAs are substrates for the lipid-biosynthesis pathway (Kennedy Pathway), which is located in the endoplasmic reticulum (ER). This pathway is responsible for membrane lipid formation as well as the biosynthesis of triacylglycerols, which constitute the seed oil. In the ER there are additional membrane-bound desaturases, which can further desaturate 18:1 to polyunsaturated fatty acids. A delta-12 desaturase (FAD2) catalyzes the insertion of a double bond into 18:1 (oleic acid), forming linoleic acid (18:2). A delta-15 desaturase (FAD3) catalyzes the insertion of a double bond into 18:2, forming linolenic acid (18:3) (FIG. 9). Thus, desaturation of oleic acid to linoleic acid (18:2) is catalyzed by FAD2 in the ER and FAD6 in the plastid, whereas linoleic acid desaturation to y-linolenic acid (C18:3, n6) is catalyzed by FAD3 in the ER and FAD7/FAD8 in the plastid.

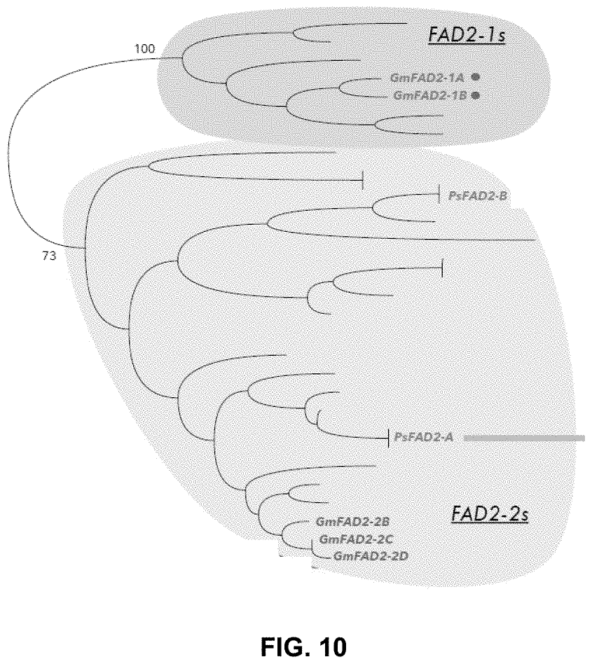

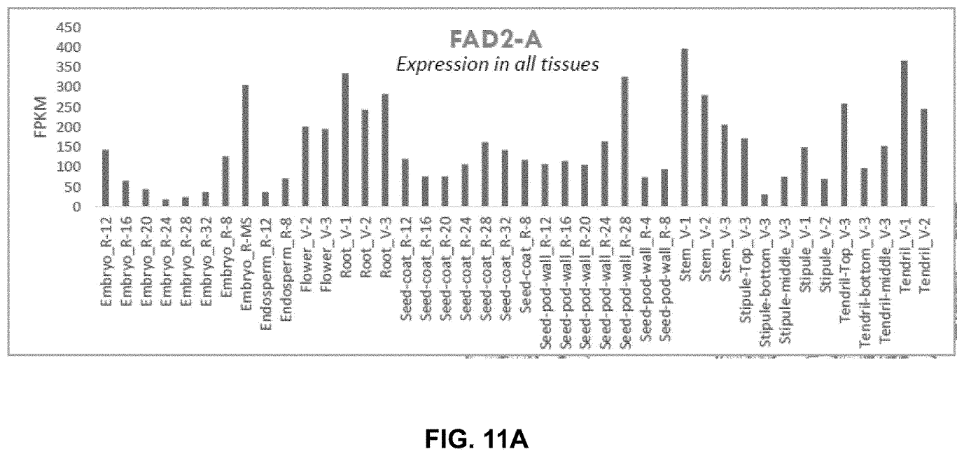

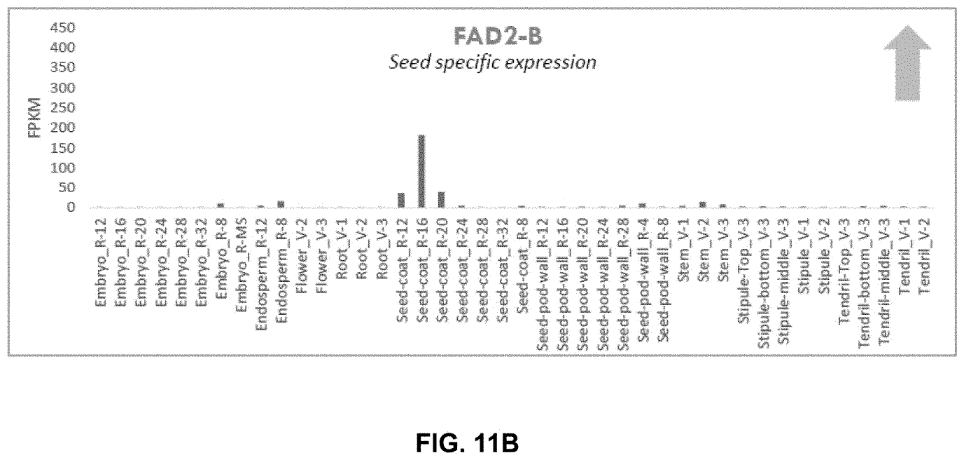

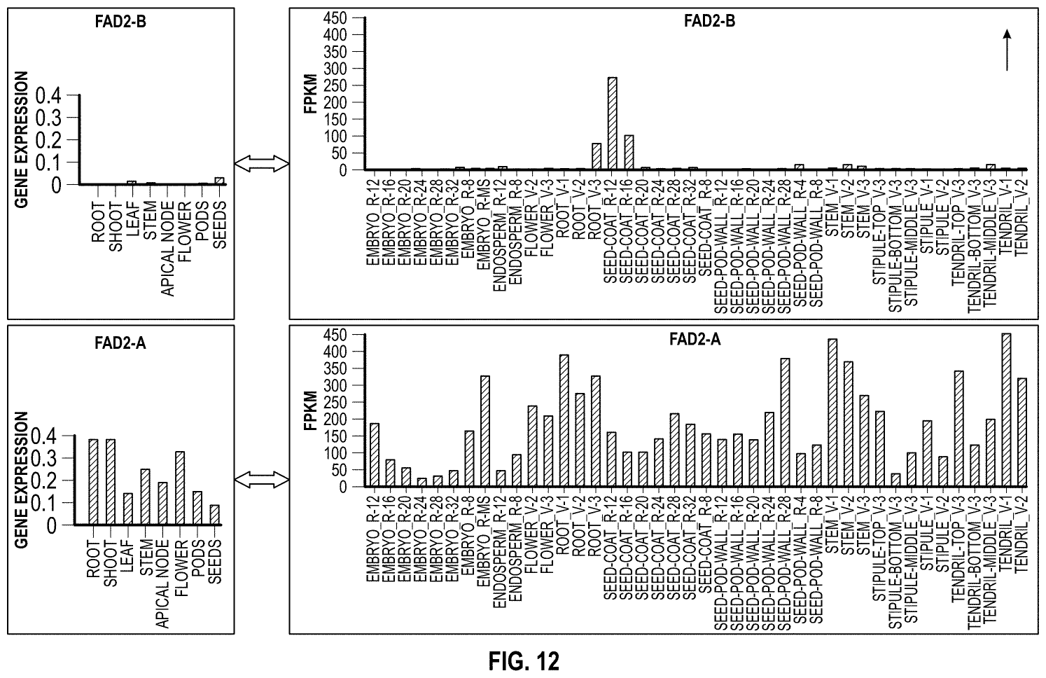

FAD2 is 1,164 bp long with an open reading frame coding for about 387 amino acids. The FAD2 gene consists of a single large intron in the 5′-untranslated region (UTR), which is evolutionarily conserved. However, the exon number may vary across the plant species, for example, Arabidopsis, castor bean, and soybean had only one exon, in contrast, Indian mustard contains two. The FAD2 gene has been classified into four types, namely, FAD2-1, FAD2-2, FAD2-3, and FAD2-4 on the basis of their site and pattern of expression. The four variations of the FAD2 gene show high sequence similarity, but show differences in their expression patterns and functions in fatty acid modification (Kongcharoensuntorn, Ph.D. thesis, University of North Texas; Denton, Tex.: 162 10). The FAD2-1 is a seed-specific desaturase that synthesizes polyunsaturated fatty acids in young seed and developing flower buds (Liu et al., Am. J. Bot. 88 92-102. 10.). FAD2-2 is expressed at a low level from vegetative stage to maturing phase during seed development (Pirtle et al., Biochim. Biophys. Acta 1522 122-129. 10). FAD2-2 is the major gene responsible for the synthesis of linoleic acid (Hernández et al., J. Agric. Food Chem. 57 6199-6206. 10). FAD2-3 and FAD2-4 synthesize mostly polyunsaturated fatty acids almost in all the tissues. A phylogenetic tree of soybean and pea FAD2 is shown in FIG. 10. This tree shows that pea and soybean FAD2-1 and FAD2-2 are distant relatives. Pea FAD2-2s include “FAD2A” (or “PsFAD2A”, “PsFAD2-A”, “FAD2-A”) and “FAD2B” (or “PsFAD2B”, “PsFAD2-B”, “FAD2-B”). FAD2 genes are expressed differently in different tissues of the plant, and the over-expression of FAD2 modifies physiological and vegetative characteristics. (Okuley et al., Plant Cell 6 147-158. 10). The differential expression of Yellow pea FAD2A and FAD2B are shown in FIGS. 11A & 11B, respectively. FIG. 12 shows differential expression of FAD2A and FAD2B in different plant tissue. The CropOS gene expression data on the right shows significantly detailed information of the FAD2A and FAD2B expression compared to the corresponding publicly available expression information shown on the left. The high definition CropOS gene expression data enables identification of the correct target tissues and the target genes.

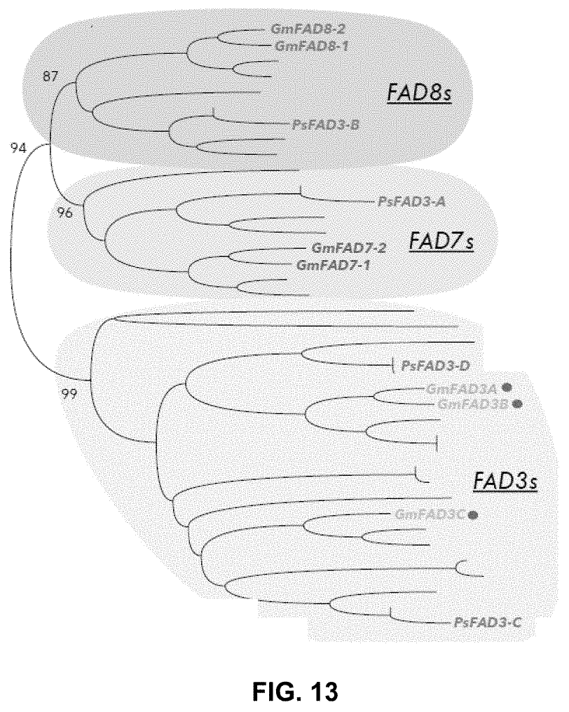

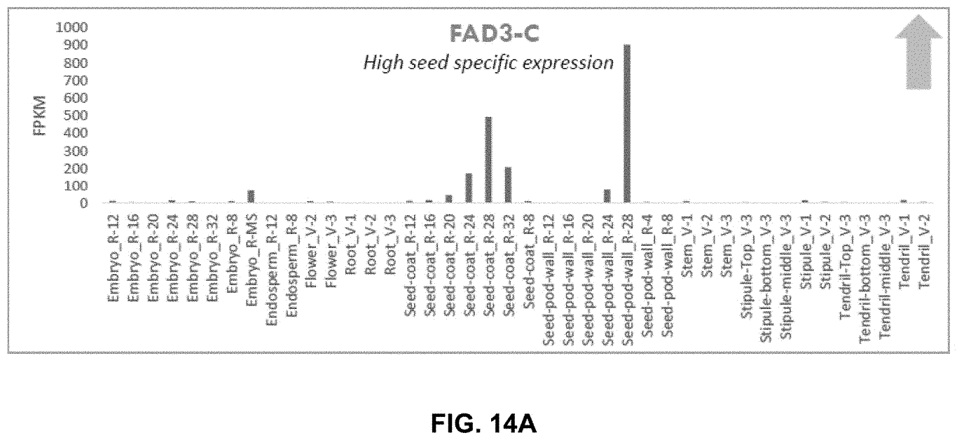

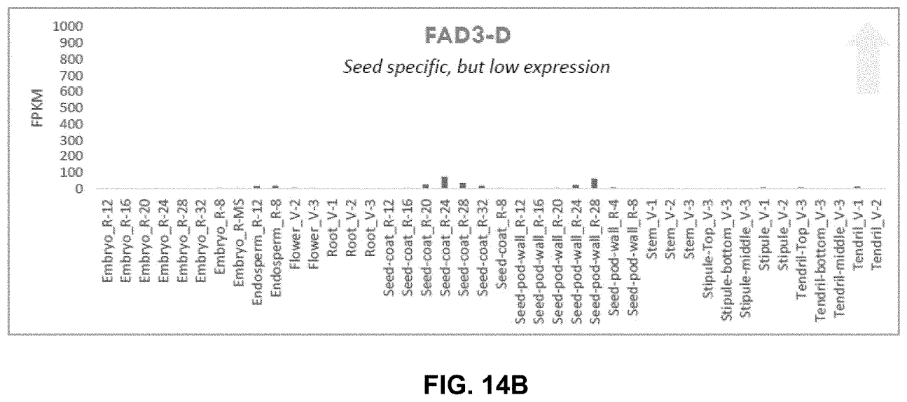

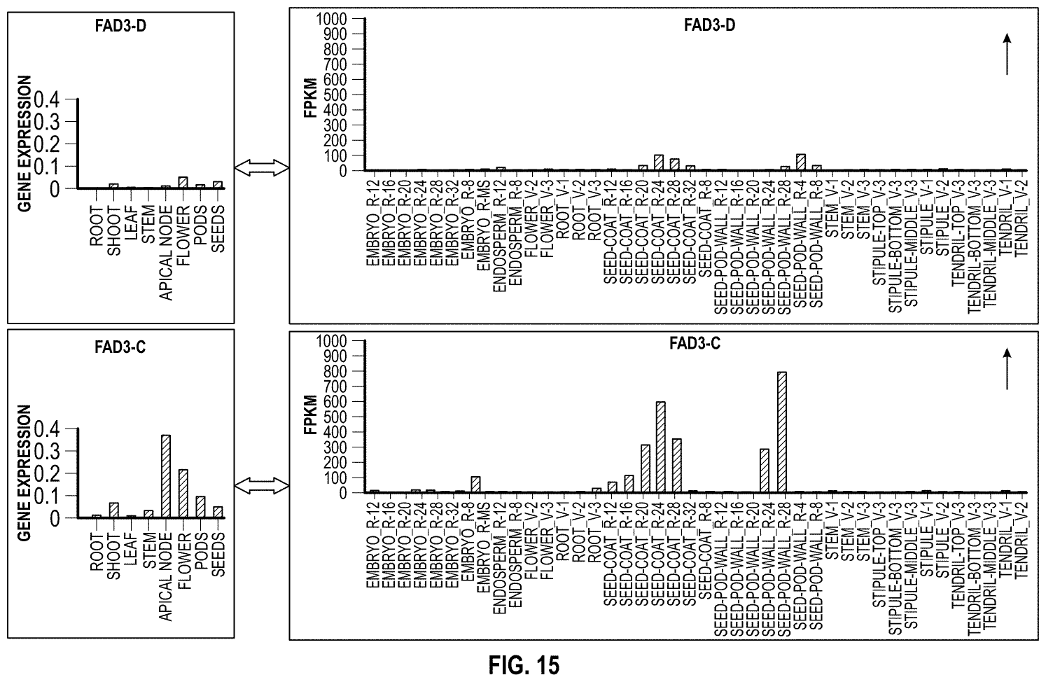

A phylogenetic tree of soybean and pea FAD3s, FAD7s, and FAD8s is shown in FIG. 13. This phylogenetic tree shows that pea FAD3s, FAD7s, and FAD8s are distant relative. Pea FAD3s include “FAD3C” (or “PsFAD3C”, “PsFAD3-C”, “FAD3-C”) and “FAD3D” (or “PsFAD3D”, “PsFAD3-D”, “FAD3-D”). Pea FAD7 includes “FAD3A” (or “PsFAD3A”, “PsFAD3-A”, “FAD3-A”). Pea FAD8 includes “FAD3B” (or “PsFAD3B”, “PsFAD3-B”, “FAD3-B”). The differential expression of yellow pea FAD3 are shown in FIGS. 14A & 14B, respectively. FIG. 14A shows differential expression of FAD3C in different plant tissue. FIG. 14B shows differential expression of FAD3D in different plant tissue. FIG. 15 shows differential expression of FAD3C and FAD3D in different plant tissue. The CropOS gene expression data on the right shows significantly detailed information of the FAD3C and FAD3D expression compared to the corresponding publicly available expression information shown on the left. The high definition CropOS gene expression data enables to identify the correct target tissues and the target genes.

Inhibition of the endogenous FAD2 gene through use of transgenes in soybeans that inhibit the expression of FAD2 has been shown to confer a desirable mid-oleic acid (18:1) phenotype (i.e. soybean seed comprising about 50% and 75% oleic acid by weight). Linoleic and linolenic acids are polyunsaturated fatty acids (PUFAs) that are essential for health and nutrition, as these cannot be synthesized in humans and have to be supplied through diet (Guan et al., Plant Mol. Biol. Rep. 30 139-148.). Despite health benefits of PUFAs, they make the edible oil more vulnerable to rancidity, decrease its flavor, and shorten its shelf life (Pandey et al., BMC Genetics 15:133. 10). The oxidative stability and nutritional value of the edible oil are dependent upon the fatty acid content of the oil, especially of oleic and linoleic acids (Cao et al., BMC Plant Biol. 13:5). Oleic acid was found to have higher oxidative stability than linoleic acid, resulting in the extension of its shelf life (Ge et al., Genet. Mol. Res. 14 17482-17488). Hence, there can be many benefits of targeting FADs, particularly, FAD2 and FAD3, in plants. Also, there is a high demand for premium quality oil rich in monounsaturated fatty acids and poor in PUFAs. Such oils are more desirable, both nutritionally and commercially. Consumption of oils rich in monounsaturated fatty acids helps to reduce cholesterol, suppresses tumor formation, and protects from inflammatory diseases. Therefore, increasing the oleic acid content in the oil is important for the development of crops to produce stable and healthy oils The desaturation of fatty acids is one of the important biochemical processes that define the quality and economic significance of the vegetable oil. Transgenes and transgenic plants that provide for inhibition of the endogenous FAD2 gene expression and a mid-oleic phenotype are disclosed in U.S. Pat. No. 7,067,722. In contrast, wild-type soybean plants that lack FAD2 inhibiting transgenes typically produce seed with oleic acid compositions of less than 20%.

Provided herein are Pisum sativum plants or plant parts comprising decreased activity of a liopoxygenase gene and the activity of the liopoxygenase gene is decreased when compared to a control plant or plant part expressing wild-type activity of the corresponding lipoxygenase gene. The Pisum sativum plants or plant parts comprise one or more insertions, substitutions, or deletions in one or more genes selected from the group consisting of LOX-2 and LOX-3.

Described herein are methods for producing plants or plant parts having improved flavor, wherein the method comprises mutating a gene encoding the LOX (e.g., LOX-2, LOX-3) protein. Thus, the methods described herein have the potential for producing a plant or plant part with altered LOX activity that could have improved flavor when compared to a control plant.

Also described herein are plants (e.g., Pisum sativum plants) wherein a gene encoding the LOX (e.g., LOX-2, LOX-3) protein has been mutated (e.g., by one or more insertions, substitutions, or deletions), resulting in loss-of-function or reduced function in the encoded LOX protein. The level of hexanal and/or hexanol in such plants can be reduced relative to a control plant that has a wild-type (WT) LOX gene and expresses fully functional LOX protein. In specific embodiments, reduction of the level of hexanal and/or hexanol can be responsible for a corresponding improvement in flavor of the plant or plant part, such as a plant protein composition (e.g., yellow pea protein concentrate) extracted from the plant or plant part.

Furthermore, provided herein are plant (e.g., Pisum sativum plants) parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, pods etc.), plant protein, plant concentrate (e.g., whole plant concentrate or plant part concentrate such as yellow pea protein concentrate), plant powder (e.g., formulated powder, such as formulated plant part powder (e.g., seed flour)), and plant biomass (e.g., dried biomass, such as crushed and/or powdered biomass) from plants containing mutation in LOX (e.g., LOX-2, LOX-3) gene, and methods of producing such plants or progeny of such plants. In some embodiments, plant parts, plant concentrate, plant biomass, and/or plant powder from such plants have reduced level of hexanal and/or hexanol compared to plant parts, plant concentrate, plant biomass, and/or plant powder from a control plant that contains an unmutated and/or a WT LOX gene. In certain instances, the plant described herein is Pisum sativum.

Also provided herein are Pisum sativum plants or plant parts comprising decreased activity of a fatty acid desaturase (FAD), and the activity of the FAD gene is decreased when compared to a control plant or plant part expressing wild-type activity of the corresponding FAD gene. The Pisum sativum plants or plant parts comprise one or more insertions, substitutions, or deletions in one or more genes selected from the group consisting of FAD2 and FAD3.

Described herein are Pisum sativum plants wherein a gene encoding the FAD (e.g., FAD2B, FAD3C, FAD3D) protein has been mutated (e.g., by one or more insertions, substitutions, or deletions), resulting in loss-of-function or reduced function in the encoded FAD protein. The level of linolenic acid in such plants can be reduced relative to a control plant that has a wild-type (WT) FAD (e.g., FAD2B, FAD3C, FAD3D) gene and/or expresses fully functional FAD (e.g., FAD2B, FAD3C, FAD3D) protein. In specific embodiments, reduction of the level of linolenic acid can be responsible for a corresponding improvement in flavor of the Pisum sativum plant or plant part, such as a plant protein composition (e.g., yellow pea protein concentrate) extracted from the plant or plant part. In specific embodiments, reduction of the level of linolenic acid in combination with the increase in oleic acid is responsible for a corresponding improvement in flavor of the Pisum sativum plant or plant part, such as a plant protein composition (e.g., yellow pea protein concentrate) extracted from the plant or plant part.

Furthermore, provided herein are Pisum sativum plant parts (e.g., juice, pulp, seed, fruit, flowers, nectar, embryos, pollen, ovules, leaves, stems, branches, bark, kernels, ears, cobs, husks, stalks, roots, root tips, anthers, pods etc.), Pisum sativum plant extract (e.g., sweetener, antioxidants, alkaloids, etc.), Pisum sativum plant protein, plant concentrate (e.g., whole plant concentrate or plant part concentrate such as yellow pea protein concentrate), plant powder (e.g., formulated powder, such as formulated plant part powder (e.g., seed flour)), and Pisum sativum plant biomass (e.g., dried biomass, such as crushed and/or powdered biomass) from Pisum sativum plants containing mutation in FAD (e.g., FAD2B, FAD3C, FAD3D) gene, and methods of producing such Pisum sativum plants or progeny of such plants. In some embodiments, Pisum sativum plant parts, plant concentrate, plant biomass, and/or plant powder from such Pisum sativum plants have reduced level of hexanal and/or hexanol compared to Pisum sativum plant parts, plant concentrate, plant biomass, and/or plant powder from a control Pisum sativum plant that contains an unmutated and/or a WT FAD (e.g., FAD2B, FAD3C, FAD3D) gene.

2.2 Plants with reduced LOX-2 Function

Provided herein are plants and plant parts with reduced LOX (e.g., LOX-2, LOX-3) function. In particular, plants and plant parts with reduced LOX (e.g., LOX-2, LOX-3) function can have a reduced level of hexanal and/or hexanol. A plant or plant part described herein (e.g., a plant or plant part containing a mutation in the LOX gene) can express a LOX (e.g., LOX-2, LOX-3) protein that comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 7, 8, 9, 25, or 26. For example, a plant or plant part described herein can have a LOX-2 protein that comprises the amino acid sequence of SEQ ID NO: 7, 8, or 9. A plant or plant part described herein can also have a LOX-3 protein that comprises the amino acid sequence of SEQ ID NO: 25 or 26.

In some embodiments, the plant has a gene that is a homolog or ortholog of the LOX-2 gene disclosed herein, and expresses a LOX-2 protein with LOX-2 function. For example, homologs of LOX-2 include, but are not limited to red clover LOX-2 (Trifohum pretense, NCBI ID: PNY17661.1), Barrel medic LOX-2 (Medicago truncatula, NCBI ID: XP_003597558.1), Chickpea LOX-2 (Cicer arietinum, NCBI ID: XP_027189582.1), Narrow-leaved blue lupine LOX-2 (Lupinus angustifolius, NCBI ID: OIW08988.1), White lupine LOX-2 (Lupinus albus, NCBI ID: KAE9585933.1), Pigeon pea LOX-2 (Cajanus cajan, NCBI ID: XP_020224319.1), Soybean LOX-2 (Glycine max, NCBI ID: NP_001237685.2), Peanut LOX-2 (Arachis hypogaea, NCBI ID: XP_025613698.1), Cowpea LOX-2 (Vigna unguiculata, NCBI ID: XP_027925673.1), Adzuki bean LOX-2 (Vigna angularis, NCBI ID: XP_017425254.1), Mung bean LOX-2 (Vigna radiate, NCBI ID: XP_014499686.1), common bean LOX-2 (Phaseolus vulgaris, NCBI ID: XP_007150486.1). The methods and compositions disclosed herein encompass reducing the function, levels, or expression of any LOX-2 gene or protein in a plant, and particularly in legumes.

A plant or plant part described herein (e.g., a plant or plant part containing a mutation in LOX gene) can express a LOX (e.g., LOX-2, LOX-3) protein that retains LOX function, either fully, or in part. LOX can catalyze the addition of molecular oxygen at either the C-9 or C-13 residue of fatty acids with a 1,4-pentadiene structure. Linoleic and linolenic acids are the most abundant fatty acids in the lipid fraction of plant membranes and are the major substrates for LOXs. As described in FIG. 1, LOX can catalyze the formation of Z, E-conjugated hydroperoxides (HPOs) from polyunsaturated fatty acids, such as linoleic and linolenic acid. In some instances, LOX (e.g., LOX-2, LOX-3) activity comprises oxidation of polyunsaturated fatty acids to produce hexanal and/or hexanol in plants or plant parts. Thus, a plant or plant part described herein may express a LOX (e.g., LOX-2, LOX-3) protein that can produce hexanal and/or hexanol in plants or plant parts by oxidation of polyunsaturated fatty acids. In some instances, a plant or plant part described herein may contain a mutation in the LOX-2 and/or LOX-3 gene, and thus express a LOX-2 and/or LOX-3 protein with reduced function or loss-of-function. Accordingly, such plant or plant part can have reduced level of hexanal and/or hexanol, as compared to a control plant or plant part, which contains a wild-type (WT) LOX-2 and/or LOX-3 gene and expresses a fully functional LOX-2 and/or LOX-3 protein.

A plant or plant part described herein can contain a mutation in a LOX (e.g., LOX-2) gene. In particular, a plant or plant part described herein can contain a LOX-2 gene that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 10. For example, a plant or plant part described herein can have a LOX-2 gene that comprises the nucleic acid sequence of SEQ ID NO: 10. A plant or plant part described herein can comprise 1-6, 2-4, 3-4, 2-5, or 3-5 (e.g., 1, 2, 3, 4, 5, or 6) copies of LOX (e.g., LOX-2) gene. In particular, a plant or plant part described herein can comprise at least 2 genes encoding a LOX-2 protein, such as 2 genes that have less than 100% (e.g., less than 100%, 99%, 98%, 97%, 96%, 95%, 94%, 93%, 92%, 91%, 90%, 89%, 88%, 87%, 86%, or 85%) sequence identity.

A plant or plant part described herein can contain a LOX-3 gene that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 27. For example, a plant or plant part described herein can have a LOX-3 gene that comprises the nucleic acid sequence of SEQ ID NO: 27. A plant or plant part described herein can comprise 1-10, 2-4, 3-4, 2-5, or 3-5 (e.g., 1, 2, 3, 4, 5, 6, 7, 8, 9 or 10) copies of LOX (e.g., LOX-3) gene. In particular, a plant or plant part described herein can comprise at least 2 genes encoding a LOX-3 protein, such as 2 genes that have less than 100% (e.g., less than 100%, 99%, 98%, 97%, 96%, 95%, 94%, 93%, 92%, 91%, 90%, 89%, 88%, 87%, 86%, or 85%) sequence identity.

Described herein are plants or plant parts, in which a gene encoding a LOX (e.g., LOX-2) protein has been mutated (e.g., by one or more insertions, substitutions, or deletions). For example, disclosed herein are plants or plant parts in which a gene encoding the LOX-2 protein has been mutated, e.g., by one or more insertions, substitutions, or deletions. In some embodiments, the one or more insertions, substitutions, or deletions are in a region that corresponds to a nucleotide region of a gene encoding the LOX-2 protein comprising nucleotides 1521 through 1531 of SEQ ID NO: 10, or nucleotides 1523 through 1530 of SEQ ID NO: 10. In some embodiments, said plants or plant parts comprise SEQ ID NO: 5 or 6. In other embodiments, the one or more insertions, substitutions, or deletions are in a region that corresponds to a nucleotide region of exon 4 of a gene encoding the LOX-2 protein comprising a nucleotide sequence set forth in SEQ ID NO: 3. In some embodiments, the one or more insertions, substitutions, or deletions are in a region that corresponds to a nucleotide region of a gene encoding the LOX-3 protein comprising nucleotides 1129 through 1156 of SEQ ID NO: 27. In some embodiments, said plants or plant parts comprise SEQ ID NO: 24. In other embodiments, the one or more insertions, substitutions, or deletions are in a region that corresponds to a nucleotide region of exon 4 of a gene encoding the LOX-3 protein comprising a nucleotide sequence set forth in SEQ ID NO: 22.

In some embodiments, the one or more insertions, substitutions, or deletions are in a region that corresponds to a nucleotide region of the gene encoding the LOX-2 protein (“LOX-2 gene”) and a region that corresponds to a nucleotide region of the gene encoding the LOX-3 protein (“the LOX-3 gene”). In some embodiments, the one or more insertions, substitutions, or deletions, or part thereof are at least partially in a region that corresponds to a nucleotide region of exon 4 of the LOX-2 gene, and in a region that corresponds to a nucleotide region of exon 4 of the LOX-3 gene. As used herein, where all or a part of an insertion, a substitution, or a deletion is “at least partially” in a certain nucleotide region, the whole part of the insertion, the substitution, or the deletion can be within the certain nucleotide region, or alternatively, can span across the certain nucleotide region and a region outside the nucleotide region. For instance, where an insertion, a substitution, or a deletion is at least partially in a certain nucleotide region corresponding to an exon, the whole part of the insertion, the substitution, or the deletion can be within the exon, or can span across the exon and a region (e.g., an intron) upstream or downstream of the exon. In some embodiments, said plants or plant parts comprise a deletion in the LOX-2 gene corresponding to nucleotides 1521 through 1531 of SEQ ID NO: 10, and a deletion in the LOX-3 nucleotides 1129 through 1156 of SEQ ID NO: 27. In some embodiments, said plants or plant parts comprise a deletion in the LOX-2 gene corresponding to nucleotides 1523 through 1530 of SEQ ID NO: 10, and a deletion in the LOX-3 nucleotides 1129 through 1156 of SEQ ID NO: 27.

In some embodiments, the one or more insertions, substitutions, or deletions are in a region that corresponds to a nucleotide region of one or more of the genes encoding the LOX-2, LOX-3, FAD2B, FAD3C, and FAD3D proteins. In some embodiments, the one or more insertions, substitutions, or deletions are in a region that corresponds to a nucleotide region of the LOX-2 gene, and in a region that corresponds to a nucleotide region of the LOX-3, FAD2B, FAD3C, and FAD3D genes. Plants or plant parts comprising one or more mutations in a FAD2 gene and/or a FAD3 gene, alone or in combination with one or more mutations in a LOX-2 gene and/or a LOX-3 gene, and methods of producing said plants or plant parts are provided herein, and described elsewhere in the present disclosure.

In many embodiments described herein, the deletion is an out-of-frame deletion. In other embodiments described herein, the deletion is an in-frame deletion.

Also provided herein are plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant protein, plant concentrate (e.g., whole plant concentrate or plant part concentrate), plant powder (e.g., formulated powder, such as formulated plant part powder (e.g., seed flour)), and plant biomass (e.g., dried biomass, such as crushed and/or powdered biomass) obtained from plants with such mutation in a LOX-2 gene. Also provided herein are seeds, such as a representative sample of seeds, from a plant of the present disclosure. In some embodiments, the plants or plant parts comprise a LOX-2 protein that has been mutated (e.g., by one or more insertions, substitutions, or deletions). In some embodiments described herein, the plants or plant parts comprise an altered LOX-2 protein that has been further mutated (e.g., by one or more insertions, substitutions, or deletions). In many embodiments described herein, the plants or plant parts comprise a LOX-2 protein that has an amino acid sequence having at least 90% sequence identity to the amino acid sequence set forth in SEQ ID NOs: 7-9. In some embodiments, the plants or plant parts comprise a LOX-2 protein that has an amino acid sequence set forth in SEQ ID NOs: 7-9. In some other embodiments, the plants or plant parts comprise a LOX-3 protein that has been mutated (e.g., by one or more insertions, substitutions, or deletions). In some embodiments described herein, the plants or plant parts comprise an altered LOX-3 protein that has been further mutated (e.g., by one or more insertions, substitutions, or deletions). In many embodiments described herein, the plants or plant parts comprise a LOX-3 protein that has an amino acid sequence having at least 90% sequence identity to the amino acid sequence set forth in SEQ ID NOs: 25-26. In some embodiments, the plants or plant parts comprise a LOX-3 protein that has an amino acid sequence set forth in SEQ ID NOs: 25-26.

In some embodiments, the gene encoding a LOX-2 protein comprises: (a) a nucleic acid sequence having at least 90% sequence identity to the nucleic acid sequence set forth in SEQ ID NO: 10, wherein said nucleic acid sequence encodes a functional LOX-2 protein; or (b) the nucleic acid sequence set forth in SEQ ID NO: 10. In some embodiments, the gene encoding a LOX-3 protein comprises: (a) a nucleic acid sequence having at least 90% sequence identity to the nucleic acid sequence set forth in SEQ ID NO: 27, wherein said nucleic acid sequence encodes a functional LOX-3 protein; or (b) the nucleic acid sequence set forth in SEQ ID NO: 27.

A plant or plant part of the present disclosure can be a crop plant or part of a crop plant. Examples of crop plants include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, or alternatively, a plant or plant part of the present disclosure can be a legume, i.e., a plant belonging to the family Fabaceae (or Leguminosae), or a part (e.g., fruit or seed) of such a plant. When used as a dry grain, the seed of a legume is also called a pulse. Examples of legume include, without limitation, beans (Phaseolus spp.), soybean (Glycine max), pea (Pisum sativum), bean (Phaseolus spp.), soybean (Glycine max), chickpea (Cicer arietinum), peanut (Arachis hypogaea), lentils (Lens culinaris, Lens esculenta), fava bean (Vicia faba), mung bean (Vigna radiata), lupins (Lupinus spp.), mesquite (Prosopis spp.), carob (Ceratonia siliqua), tamarind (Tamarindus indica), alfalfa (Medicago sativa), and clover (Trifolium spp.). Additionally, or alternatively, a plant or plant part of the present disclosure can be an oilseed plant (e.g., canola (Brassica napus), cotton (Gossypium sp.), camelina (Camelina sativa) and sunflower (Helianthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). For example, a plant or plant part of the present disclosure can be Pisum sativum or a part of Pisum sativum.

In certain instances, mutations in any LOX gene in a plant, plant part, or protein composition obtained from plant or plant part can be identified by a diagnostic method described herein. Such diagnostic methods may comprise use of primers for detecting mutation in LOX gene. For example, forward primer 13062 (SEQ ID NO: 13) and reverse primer 13057 (SEQ ID NO: 14) can be used for detection of mutation in LOX-2 gene. The forward primer 13091 (SEQ ID NO: 59) and reverse primer 042 (SEQ ID NO: 60) can be used for detection of mutation in LOX-3 gene. In certain instances, a kit comprising a set of primers can be used for detecting mutation of LOX gene in plants, plant parts, or protein composition obtained from plants or plant parts. For example, a kit comprising forward primer 13062 (SEQ ID NO: 13) and reverse primer 13057 (SEQ ID NO: 14) can be used for detection of mutation in LOX-2 gene in plants, plant parts, or protein composition obtained from plants or plant parts. In some instances, the forward primer 13091 (SEQ ID NO: 59) and reverse primer 042 (SEQ ID NO: 60) can be used for detection of mutation in LOX-3 gene in plants, plant parts, or protein composition obtained from plants or plant parts.

(i) Plants with Reduced Expression of Full-Length LOX Protein

A plant or plant part of the present disclosure can have reduced expression of a LOX (e.g., LOX-2, LOX-3) protein, as compared to a control plant or plant part, such as a plant or plant part that contains an unmutated and/or WT LOX gene. In particular, a plant or plant part that contains a mutated LOX gene can have reduced expression of a full length LOX (e.g., LOX-2, LOX-3) protein, as compared to a control plant or plant part. For example, a plant or plant part that contains a mutated LOX-2 gene can have reduced expression of full length LOX-2 protein, as compared to a control plant or plant part. A control plant or plant part can be a plant or plant part that has a full-length or wild-type LOX-2 gene. In some embodiments, a plant or plant part that contains a mutated LOX-3 gene can have reduced expression of full length LOX-3 protein, as compared to a control plant or plant part. A control plant or plant part can be a plant or plant part that has a full-length or wild-type LOX-3 gene. For example, a control plant or plant part can be a plant or plant part before a LOX gene in the plant or plant part is mutated. Thus, a control plant or plant part may express a WT LOX (e.g., LOX-2, LOX-3) gene. A control plant of the present disclosure may be grown under the same environmental conditions (e.g., same or similar temperature, humidity, air quality, soil quality, water quality, and/or pH conditions) as a plant that contains a mutated LOX gene. A plant or plant part containing a mutated LOX gene can have reduced expression of a LOX (e.g., full length LOX) protein, as compared to a control plant or plant part, when the plant or plant part with the mutated LOX gene is grown under the same environmental conditions as the control plant or plant part. In some embodiments, expression of LOX (e.g., full length LOX) protein in a plant or plant part with a mutated LOX gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, expression of LOX (e.g., full length LOX) protein in a plant or plant part, which contains a mutated LOX gene, can be reduced by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In specific embodiments, the LOX protein is a LOX-2 protein. In other embodiments, the LOX protein is a LOX-3 protein.

Plant parts, plant extracts, plant protein, plant concentrate, plant powder, and/or plant biomass, which is obtained from plants containing a mutated LOX gene, can have reduced expression of a LOX (e.g., LOX-2, LOX-3) protein or LOX (e.g., LOX-2, LOX-3) activity, as compared to plant parts, plant extracts, protein, plant concentrate, plant powder, and/or plant biomass obtained from a control plant. In particular, plant part, plant extract, plant protein, plant concentrate, plant powder, and/or plant biomass obtained from plants with a mutated LOX gene can have reduced expression of a full length LOX (e.g., LOX-2, LOX-3) protein, as compared to plant part, plant extract, plant protein, plant concentrate, plant powder, and/or plant biomass obtained from a control plant. In some embodiments, plant part, plant extract, plant protein, plant concentrate, plant powder, and/or plant biomass obtained from a plant with a mutated LOX-3 gene can have reduced expression of full length LOX-3 protein, as compared to plant part, plant extract, plant protein, plant concentrate, plant powder, and/or plant biomass obtained from a control plant. For example, plant part, plant extract, plant protein, plant concentrate, plant powder, and/or plant biomass obtained from a plant with a mutated LOX-2 gene can have reduced expression or reduced levels of full length LOX-2 protein, as compared to plant part, plant extract, plant protein, plant concentrate, plant powder, and/or plant biomass obtained from a control plant. Plant part, plant extract, plant protein, plant concentrate, plant powder, and/or plant biomass obtained from a plant with a mutated LOX gene can have reduced expression or reduced levels of LOX (e.g., a full length LOX) protein, as compared to plant part, plant extract, plant protein, plant concentrate, plant powder, and/or plant biomass obtained from a control plant, when the plant with the mutated LOX gene is grown under the same environmental conditions as the control plant. In some embodiments, expression or levels of LOX (e.g., full length LOX) protein in plant part, plant extract, plant protein, plant concentrate, plant powder, and/or plant biomass obtained from a plant with a mutated LOX gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to plant part, plant extract, plant protein, plant concentrate, plant powder, and/or plant biomass obtained from a control plant. In some embodiments, expression of LOX-2 and LOX-3 proteins in plant part, plant extract, plant protein, plant concentrate, plant powder, and/or plant biomass obtained from a plant with a mutated LOX-2 and LOX-3 genes can each be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to the plant part, plant extract, plant protein, plant concentrate, plant powder, and/or plant biomass obtained from a control plant comprising WT LOX-2 and LOX-3 genes. Additionally, or alternatively, expression or levels of LOX (e.g., full length LOX-2 and/or LOX-3) protein in plant part, plant extract, plant protein, plant concentrate, plant powder, and/or plant biomass obtained from a plant with a mutated LOX gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to plant part, plant extract, plant protein, plant concentrate, plant powder, and/or plant biomass obtained from a control plant. In specific embodiments, the LOX protein is a LOX-2 protein. In some embodiments, the LOX protein is a LOX-3 protein. In some embodiments, the LOX protein is a LOX-2 and a LOX-3 protein.

Expression of a LOX (e.g., LOX-2, LOX-3) protein, such as a full length LOX protein, in a plant, plant part, plant extract, plant protein, plant concentrate, plant powder, and/or plant biomass can be determined by one or more standard methods known in the art. In some embodiments, expression of a LOX protein can be determined by western blot analysis of a protein sample obtained from a plant, plant part, plant extract, plant protein, plant concentrate, plant powder, and/or plant biomass by using an antibody directed to the LOX protein. For example, expression of a full length LOX protein can be determined by western blot analysis of a protein sample obtained from a plant, plant part, plant extract, plant protein, plant concentrate, plant powder, and/or plant biomass by using an antibody directed to the full length LOX protein. Details of such procedure has been outlined in the Examples section of the present disclosure.

(ii) Plants with Lss-of-Function or Reduced Function in LOX Protein

A plant or plant that contains a mutated LOX gene can have loss-of-function or reduced function in the encoded LOX (e.g., LOX-2, LOX-3) protein, as compared to a control plant or plant part. For example, a plant or plant part that contains a mutated LOX-2 gene can have loss-of-function or reduced function (i.e., reduced LOX-2 activity and/or reduced LOX-3 activity) in the encoded LOX-2 protein, as compared to a control plant or plant part. A control plant or plant part can be a plant or plant part that does not contain a mutation in the LOX gene and/or contain a WT

LOX gene. For example, a control plant or plant part can be a plant or plant part before a LOX gene in the plant or plant part is mutated. Thus, a control plant or plant part may express WT LOX (e.g., LOX-2, LOX-3) gene. A control plant of the present disclosure may be grown under the same environmental conditions (e.g., same or similar temperature, humidity, air quality, soil quality, water quality, and/or pH conditions) as a plant that contains the mutated LOX gene. A plant or plant part that contains a mutated LOX gene can have loss-of-function or reduced function in the encoded LOX protein, as compared to a control plant or plant part, when the plant or plant part with a mutated LOX gene is grown under the same environmental conditions as the control plant or plant part. In some embodiments, LOX activity in a plant or plant part with a mutated LOX gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In some embodiments, activity of LOX-2 and LOX-3 in a plant or plant part with a mutated LOX-2 and LOX-3 genes can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part comprising WT LOX-2 and LOX-3 proteins. Additionally, or alternatively, LOX activity in a plant or plant part with a mutated LOX gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part.

Also, plant part, plant extract, plant protein, plant concentrate, plant powder, and/or plant biomass obtained from plants with a mutated LOX gene can have loss-of-function or reduced function of encoded LOX (e.g., LOX-2, LOX-3) protein, as compared to plant part, plant extract, plant protein, plant concentrate, plant powder, and/or plant biomass obtained from a control plant. For example, plant part, plant extract, plant protein, plant concentrate, plant powder, and/or plant biomass obtained from a plant with a mutated LOX-2 gene can have loss-of-function or reduced function of encoded LOX-2 protein, as compared to plant part, plant extract, plant protein, plant concentrate, plant powder, and/or plant biomass obtained from a control plant. In some embodiments, plant part, plant extract, plant protein, plant concentrate, plant powder, and/or plant biomass obtained from a plant with a mutated LOX-3 gene can have loss-of-function or reduced function of encoded LOX-3 protein, as compared to plant part, plant extract, plant protein, plant concentrate, plant powder, and/or plant biomass obtained from a control plant. Plant part, plant extract, plant protein, plant concentrate, plant powder, and/or plant biomass obtained from a plant with a mutated LOX gene can have loss-of-function or reduced function in the encoded LOX protein, as compared to plant part, plant extract, plant protein, plant concentrate, plant powder, and/or plant biomass obtained from a control plant, when the plant with a mutated LOX gene is grown under the same environmental conditions as the control plant. In some embodiments, function of encoded LOX protein in plant part, plant extract, plant protein, plant concentrate, plant powder, and/or plant biomass obtained from a plant with a mutated LOX gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to plant part, plant extract, plant protein, plant concentrate, plant powder, and/or plant biomass obtained from a control plant. In some embodiments, function of encoded LOX-2 and LOX-3 proteins in plant part, plant extract, plant protein, plant concentrate, plant powder, and/or plant biomass obtained from a plant with a mutated LOX-2 and LOX-3 genes can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part comprising WT LOX-2 and LOX-3 proteins. Additionally, or alternatively, function of encoded LOX protein in plant part, plant extract, plant protein, plant concentrate, plant powder, and/or plant biomass obtained from a plant with a mutated LOX gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to plant part, plant extract, plant protein, plant concentrate, plant powder, and/or plant biomass obtained from a control plant. In specific embodiments, the LOX protein is a LOX-2 protein. In some embodiments, the LOX protein is a LOX-3 protein. Function of encoded LOX (e.g., LOX-2, LOX-3) protein in a plant, plant part, plant extract, plant protein, plant concentrate, plant powder, and/or plant biomass can be determined by one or more standard methods known in the art. In some embodiments, function of encoded LOX protein can be determined by assessing enzyme activity of LOX in a protein sample obtained from a plant, plant part, plant extract, plant protein, plant concentrate, plant powder, and/or plant biomass. LOX enzyme activity can be determined by measuring fluorescence signal generated by the reaction of a fluorescent probe with oxidized fatty acid, an intermediate that is produced when LOX protein acts on a substrate of LOX. For example, enzyme activity of LOX-2 protein can be determined by using linolenic acid as a substrate. Details of such procedure has been outlined in the Examples section of the present disclosure.

(iii) Plants with Reduced Level of Hexanal and/or Hexanol

Provided herein are plants and plant parts having mutation in a LOX (e.g., LOX-2, LOX-3) gene and having decreased levels of volatile compounds, e.g., hexanal, 1-hexanol, pentanal, 1-pentanol, 1-penten-3-ol, heptanal, 1-heptanol, octanal 2-octenal (E), 1-octen-3-ol, 1-octanol, furan, 2-pentyl, and nonanal. A “plant part”, as used herein, refers to any component of a plant, such as seed, juice, pulp, fruit, flowers, nectar, embryos, pollen, ovules, leaves, stems, branches, kernels, stalks, roots, root tips, anthers. In some embodiments, levels of hexanal, hexanol, and/or other volatile compounds (e.g., pentanal, 1-pentanol, 1-penten-3-ol, heptanal, 1-heptanol, octanal 2-octenal (E), 1-octen-3-ol, 1-octanol, furan, 2-pentyl, nonanal) in a plant or plant part with a mutated LOX-2 and/or LOX-3 gene, or plant extract, plant protein, plant concentrate, plant powder, and/or plant biomass obtained from such plants or plant parts can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, plant extract, plant protein, plant concentrate, plant powder, and/or plant biomass. In some embodiments, levels of hexanal in a plant or plant part with a mutated LOX-2 and/or LOX-3 gene, or plant extract, plant protein, plant concentrate, plant powder, and/or plant biomass obtained from such plants or plant parts can be reduced by at least 70% as compared to a control plant, plant part, plant extract, plant protein, plant concentrate, plant powder, and/or plant biomass. In some embodiments, levels of 1-hexanol in a plant or plant part with a mutated LOX-2 and/or LOX-3 gene, or plant extract, plant protein, plant concentrate, plant powder, and/or plant biomass obtained from such plants or plant parts can be reduced by at least 80% as compared to a control plant, plant part, plant extract, plant protein, plant concentrate, plant powder, and/or plant biomass. The amount or level of hexanal, hexanol and/or other volatile compounds in a plant, plant part, plant extract, plant protein, plant concentrate, plant powder, and/or plant biomass can be determined by one or more standard methods known in the art. In some embodiments, amount or level of hexanal, hexanol, and/or other volatile compounds in a plant, plant part, plant extract, plant protein, plant concentrate, plant powder, and/or plant biomass is determined by Solid-Phase Micro-Extraction (SPME) and Gas Chromatography (GC). Details of such procedure has been outlined in the Examples section of the present disclosure. Mutating a gene encoding a LOX (e.g., LOX-2, LOX-3) protein can improve flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky) of a plant or plant part by reducing the level of hexanal and/or hexanol in such plant or plant part. Thus, a plant or plant part with a mutated LOX gene can have improved flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky) compared to a control plant or plant part.

Flavor characteristics of plant, plant part, or protein composition obtained from plant or plant part may refer to taste or aroma of the plant, plant part, or protein composition. Aroma relates to the ratios and intensities of volatile compounds, such as hexanol, 1-hexanal, pentanal, 1-pentanol, 1-penten-3-ol, heptanal, 1-heptanol, octanal 2-octenal (E), 1-octen-3-ol, 1-octanol, furan, 2-pentyl, and nonanal in plant, plant part, or protein composition obtained from plant or plant part. Volatile compounds that contribute to flavor characteristics (e.g., aspects described as overall aroma, overall flavor impact, beany yellow pea, pyrazine, cereal grain, green grassy green pea, nutty, cardboard, malty, salt, bitter, umami, astringent, or chalky) of plant, plant part, or protein composition obtained from plant or plant part can be quantified by using Gas Chromatography-Mass Spectroscopy (GC-MS), a lab-based technique which helps to separate and identify compounds in their gaseous forms based on their masses. In certain instances, to correlate these instrumental measurements to consumer perception, two major methods of sensory evaluation are used: consumer testing and descriptive analysis. Consumer testing includes subjective data about the preferences of a large group of untrained tasters (usually more than 100 panelists), while descriptive analysis includes questionnaires for a panel of 8-12 trained tasters who are able to rate specific attributes related to flavor or aroma. Methods for determining flavor characteristic of plants and plant parts is described in the art, e.g., by Barrett et al. (Critical Reviews in Food Science and Nutrition, 50(5): 369-389 (2010)) and Hallowell et al. (Chem Senses, 41(3):249-259 (2016)). In certain instances, flavor characteristics of plant, plant part, or protein composition obtained from plant or plant part can be determined by a flavor panel experiment. Such flavor panel experiment may use instrumental measurements, sensory testing, or a combination thereof. Plant, plant part, or protein composition that scores higher (as compared to a suitable control) in such flavor panel experiments can be considered to have improved flavor characteristics. For example, in a flavor panel experiment, a plant or plant part containing mutation in LOX-2 and/or LOX-3 gene can score higher compared to a control plant or plant part (e.g., plant or plant part that does not contain mutation in LOX-2 and/or LOX-3 gene), and thus can be considered to have improved flavor characteristics compared to the control plant or plant part.

A control plant or plant part can be a plant or plant part that does not contain a mutated LOX gene. For example, a control plant or plant part can be a plant or plant part before LOX gene in the plant or plant part is mutated. Thus, a control plant or plant part may express WT LOX (e.g., LOX-2, LOX-3) gene. A control plant of the present disclosure may be grown under the same environmental conditions (e.g., same or similar temperature, humidity, air quality, soil quality, water quality, and/or pH conditions) as a plant with a mutated LOX gene. A plant or plant part with a mutated LOX gene can have improved flavor characteristics, as compared to a control plant or plant part, when the plant or plant part with a mutated LOX gene is grown under the same environmental conditions as the control plant or plant part. Improved flavor characteristics of a plant or plant part with a mutated LOX gene can result from reduced level of hexanal and/or hexanol in such plants or plant parts. A plant or plant part with a mutated LOX gene can have reduced level of hexanal and/or hexanol, as compared to a control plant or plant part, when the plant or plant part with a mutated LOX gene is grown under the same environmental conditions as the control plant or plant part.

Plants or plant parts having a mutated LOX gene (e.g., LOX-2, LOX-3) can have characteristics provided herein, e.g., reduced level or activity of the LOX gene (e.g., LOX-2, LOX-3), reduced level of hexanal and/or 1-hexanol, improved flavor characteristics, and have no significant decrease (e.g., no statistically significant decrease, no more than 20% decrease) in yield (i.e., seed or plant yield) or total protein content as compared to a control plant, plant part (e.g., wild type, having no mutation). Plants or plant parts having a mutated LOX gene and/or FAD gene can have yields and/or total protein content of at least 80% (e.g., 80%, 85%, 90%, 95%, 99%, 100%, or more) as compared to a control plant or plant part. Yield can be measured and expressed by any means known in the art. In specific embodiments, yield is measured by seed weight or volume in a given harvest area. Protein content can be measured and expressed by any means known in the art, for example by protein extraction and quantitation (e.g., BCA protein assay, Lowry protein assay, Bradford protein assay), spectroscopy, near-infrared reflectance (NIR) (e.g., analyzing 700-2500 nm), and nuclear magnetic resonance spectrometry (NMR).

Plant parts, plant extracts, plant protein, plant concentrate, plant powder, and/or plant biomass obtained from a plant with a mutated LOX (e.g., LOX-2, LOX-3) gene can have improved flavor characteristics compared to plant part, plant extract, plant protein, plant concentrate, plant powder, and/or plant biomass obtained from a control plant. Plant part, plant extract, plant protein, plant concentrate, plant powder, and/or plant biomass obtained from a plant with a mutated LOX gene can have improved flavor characteristics, as compared to plant part, plant extract, plant protein, plant concentrate, plant powder, and/or plant biomass obtained from a control plant, when the plant with a mutated LOX gene is grown under the same environmental conditions as the control plant. Improved flavor characteristics of plant part, plant extract, plant protein, plant concentrate, plant powder, and/or plant biomass obtained from a plant with a mutated LOX gene can result from reduced level of hexanal and/or hexanol in such plant part, plant extract, plant protein, plant concentrate, plant powder, and/or plant biomass. Plant part, plant extract, plant protein, plant concentrate, plant powder, and/or plant biomass obtained from a plant with a mutated LOX gene can have reduced level of hexanal and/or hexanol, as compared to plant part, plant extract, plant protein, plant concentrate, plant powder, and/or plant biomass obtained from a control plant, when the plant with a mutated LOX gene is grown under the same environmental conditions as the control plant.

(iv) Plant Products with Reduced Level of Hexanal, Hexanol, and/or Linolenic Acid

Also provided herein are plant products produced from plants or plant parts provided herein (e.g., having mutated LOX gene). “Plant products”, as used herein, refers to any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein. Plant parts and plant products provided herein can be intended for human or animal consumption.

The plant products provided herein can comprise reduced level of hexanal, hexanol, and/or linolenic acid and/or one or more mutated nucleic acid molecules (e.g., mutated LOX gene) of the present disclosure. In specific embodiments, provided herein are a protein composition and oil, such as a protein composition or oil obtained (e.g., extracted or isolated) from a plant that contains mutated LOX gene. In particular, provided herein is a protein composition or oil obtained from a pea plant (Pisum sativum) that contains mutated LOX-2 and/or LOX-3 gene.

As used herein, a “protein product” or “protein composition” refers to any protein composition or product isolated, extracted, and/or produced from plants or plant parts (e.g., seed) and includes isolates, concentrates, and flours, e.g., soy protein composition, pea protein composition, soy protein concentrate (SPC), pea protein concentrate (PPC), soy protein isolate (SPI), pea protein isolate (PPI), soy flour, pea flour, flake, white flake, texturized vegetable protein (TVP), or textured soy protein (TSP)). A protein composition can be a concentrated protein solution (e.g., yellow pea protein concentrate solution) in which the protein is in a higher concentration than the protein in the plant from which the protein composition is derived. The protein composition can comprise multiple proteins as a result of the extraction or isolation process. In specific embodiments, the protein composition can further comprise stabilizers, excipients, drying agents, desiccating agents, anti-caking agents, or any other ingredient to make the protein fit for the intended purpose. The protein composition can be a solid, liquid, gel, or aerosol and can be formulated as a powder. The protein composition can be extracted in a powder form from a plant and can be processed and produced in different ways, such as: (i) as an isolate—through the process of wet fractionation, which has the highest protein concentration; (ii) as a concentrate—through the process of dry fractionation, which are lower in protein concentration; and/or (iii) in textured form—when it is used in food products as a substitute for other products, such as meat substitution (e.g. a “meat” patty). Protein isolate can be derived from defatted soy flour with a high solubility in water, as measured by the nitrogen solubility index (NSI). The aqueous extraction is carried out at a pH below 9. The extract is clarified to remove the insoluble material and the supernatant liquid is acidified to a pH range of 4-5. The precipitated protein-curd is collected and separated from the whey by centrifuge. The curd can be neutralized with alkali to form the sodium proteinate salt before drying. Protein concentrate can be produced by immobilizing the soy globulin proteins while allowing the soluble carbohydrates, whey proteins, and salts to be leached from the defatted flakes or flour. The protein is retained by one or more of several treatments: leaching with 20-80% aqueous alcohol/solvent, leaching with aqueous acids in the isoelectric zone of minimum protein solubility, pH 4-5; leaching with chilled water (which may involve calcium or magnesium cations), and leaching with hot water of heat-treated defatted protein meal/flour (e.g., soy meal/flour). Any of the process provided herein can result in a product that is 70% protein, 20% carbohydrates (2.7 to 5% crude fiber), 6% ash and about 1% oil, but the solubility may differ. As an example, one ton (t) of defatted soybean flakes can yield about 750 kg of soybean protein concentrate. “Texturized vegetable protein” (TVP), “Textured vegetable protein”, also referred to as “textured soy protein” (TSP), soy meat, or soya chunks refers to a defatted plant (e.g., soy) flour product, a by-product of extracting plant (e.g., soybean) oil. It can be used as a meat analogue or meat extender. It is quick to cook, with a protein content comparable to certain meats. TVP can be produced from any protein-rich seed meal left over from vegetable oil production. A wide range of pulse seeds other than soybean, such as lentils, peas, and fava beans, or peanut may be used for TVP production. TVP can be made from high protein (e.g., 50%) soy isolate, flour, or concentrate, and can also be made from cottonseed, wheat, and oats. It is extruded into various shapes (chunks, flakes, nuggets, grains, and strips) and sizes, exiting the nozzle while still hot and expanding as it does so. The defatted thermoplastic proteins are heated to 150-200° C., which denatures them into a fibrous, insoluble, porous network that can soak up as much as three times its weight in liquids. As the pressurized molten protein mixture exits the extruder, the sudden drop in pressure causes rapid expansion into a puffy solid that is then dried. As much as 50% protein when dry, TVP can be rehydrated at a 2:1 ratio, which drops the percentage of protein to an approximation of ground meat at 16%. TVP can be used as a meat substitute. When cooked together, TVP can help retain more nutrients from the meat by absorbing juices normally lost. Also provided herein are methods of isolating, extracting, or preparing any of the protein compositions or protein products provided herein from plants or plant parts.

Plant parts (e.g., seeds) and plant products (e.g., plant biomass, seed compositions, protein compositions, food and/or beverage products) produced by the methods provided herein can be meant for consumption by agricultural animals or for use as feed in an agriculture or aquaculture system. In specific embodiments, plant parts and plant products produced according to the methods provided herein include animal feed (e.g., roughages—forage, hay, silage; concentrates—cereal grains, soybean cake) intended for consumption by bovine, porcine, poultry, lambs, goats, or any other agricultural animal. In some embodiments, plant parts and plant products produced according to the methods include aquaculture feed for any type of fish or aquatic animal in a farmed or wild environment including, without limitation, trout, carp, catfish, salmon, tilapia, crab, lobster, shrimp, oysters, clams, mussels, and scallops.

A protein composition or oil obtained (i.e., extracted or isolated) from a plant with a mutated LOX (e.g., LOX-2, LOX-3) gene can have improved flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky) compared to protein composition or oil obtained from a control plant. Protein composition or oil obtained from a plant with a mutated LOX-2 and/or LOX-3 gene can have improved flavor characteristics, as compared to protein composition or oil obtained from a control plant, when the plant with a mutated LOX-2 and/or LOX-3 gene is grown under the same environmental conditions as the control plant. Improved flavor characteristics of protein compositions or oil obtained from a plant with a mutated LOX-2 and/or LOX-3 gene can result from reduced level of hexanal, hexanol and/or linolenic acid in such protein composition or oil. Protein composition or oil obtained from a plant with a mutated LOX-2 and/or LOX-3 gene can have reduced level of hexanal, hexanol, and/or linolenic acid, as compared to protein composition or oil obtained from a control plant, when the plant with mutated LOX-2 and/or LOX-3 gene is grown under the same environmental conditions as the control plant. In some embodiments, level of hexanal, hexanol, other volatile compounds (e.g., pentanal, 1-pentanol, 1-penten-3-ol, heptanal, 1-heptanol, octanal 2-octenal (E), 1-octen-3-ol, 1-octanol, furan, 2-pentyl, nonanal), and/or linolenic acid in protein composition or oil obtained from a plant with a mutated LOX-2 and/or LOX-3 gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to protein composition or oil obtained from a control plant. Additionally, or alternatively, level of hexanal, hexanol, other volatile compounds (e.g., pentanal, 1-pentanol, 1-penten-3-ol, heptanal, 1-heptanol, octanal 2-octenal (E), 1-octen-3-ol, 1-octanol, furan, 2-pentyl, nonanal), and/or linolenic acid in protein composition or oil obtained from a plant with a mutated LOX-2 and/or LOX-3 gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to protein composition or oil obtained from a control plant. In specific embodiments, level of hexanal in the protein composition or oil provided herein is reduced by at least 70% as compared to protein composition or oil obtained from a control plant. In specific embodiments, level of 1-hexanol in the protein composition or oil provided herein is reduced by at least 80% as compared to protein composition or oil obtained from a control plant. In specific embodiments, level of linolenic acid in the protein composition or oil provided herein is reduced by at least 50% as compared to protein composition or oil obtained from a control plant.

Protein composition or oil obtained from a Pisum sativum plant with a mutated LOX-2 and/or LOX-3 gene can have reduced level of linolenic acid, as compared to protein composition or oil obtained from a control plant, when the plant with ma mutated LOX-2 and/or LOX-3 gene is grown under the same environmental conditions as the control plant. In some embodiments, level of linolenic acid in protein composition or oil obtained from a plant with a mutated LOX-2 and/or LOX-3 gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to protein composition or oil obtained from a control plant. Additionally, or alternatively, level of linolenic acid in protein composition or oil obtained from a plant with a mutated LOX-2 and/or LOX-3 gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to protein composition or oil obtained from a control plant.

Also provided herein are food and/or beverage products containing a protein composition or oil described herein, such as a protein composition (e.g., yellow pea protein concentrate) or oil obtained from a plant with a mutated LOX gene. Such food and/or beverage products include, without limitation, protein shakes, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc.). A food and/or beverage product that contains a protein composition or oil obtained from a plant with a mutated LOX gene can have improved flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky), compared to a similar or comparable food and/or beverage product that contains a protein composition or oil obtained from a control plant.

### 2.3 Reducing LOX-2 Activity to Improve Flavor Characteristics

Provided herein are methods for reducing the function and/or expression of a LOX (e.g., LOX-2, LOX-3) protein in a plant or plant part. In particular, methods of the present disclosure can reduce function and/or expression of LOX protein in a plant or plant part by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, methods of the present disclosure can reduce expression and/or function of LOX protein in a plant or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In specific embodiments, the LOX protein is a LOX-2 protein. In some embodiments, the LOX protein is a LOX-3 protein. In some embodiments, methods provided herein comprise decreasing the function and/or expression LOX-2 gene in the plant with a mutant LOX-2 gene. In some embodiments, methods provided herein comprise decreasing the function and/or expression of LOX-3 gene in the plant with a mutant LOX-3. In some embodiments, methods provided herein comprise decreasing the function and/or expression of LOX-2 and LOX-3 genes in the plant with a mutant LOX-2 and LOX-3 genes.

Lipoxygenases (LOXs), a type of non-heme iron-containing dioxygenase, are ubiquitous enzymes in plants. LOX can catalyze the addition of molecular oxygen at either the C-9 or C-13 residue of fatty acids with a 1,4-pentadiene structure. Linoleic and linolenic acids are the most abundant fatty acids in the lipid fraction of plant membranes and are the major substrates for LOXs. As described in FIG. 1, LOX can catalyze the formation of Z, E-conjugated hydroperoxides (HPOs) from polyunsaturated fatty acids, such as linoleic and linolenic acid. In some instances, activity of a LOX, such as LOX-2, and/or LOX-3 can oxidate polyunsaturated fatty acids to produce hexanal and/or hexanol in plants or plant parts. In some instances, activity of a LOX, such as LOX-2, and/or LOX-3 can oxidate polyunsaturated fatty acids to produce 1-octen-3-ol in plants or plant parts. 1-Octen-3-ol is a secondary alcohol derived from a hydride of oct-1-ene and formed during oxidative breakdown of linolenic acid. It is produced by several plants and fungi and is renowned for its strong smell of mushrooms (i.e., off flavor), being a major component of mushroom volatiles. Thus, reducing the activity of LOX (e.g., LOX-2, LOX-3) protein can reduce the level of hexanal, hexanol, 1-octen-3-ol, and/or other volatile compounds (e.g., pentanal, 1-pentanol, 1-penten-3-ol, heptanal, 1-heptanol, octanal 2-octenal (E), 1-octanol, furan, 2-pentyl, nonanal) in a plant or plant part. Accordingly, in some instances, provided herein are methods for reducing the level of hexanal, hexanol, 1-octen-3-ol, and/or other volatile compounds (e.g., pentanal, 1-pentanol, 1-penten-3-ol, heptanal, 1-heptanol, octanal 2-octenal (E), 1-octanol, furan, 2-pentyl, nonanal) in a plant or plant part. In particular, methods comprise decreasing the hexanal, hexanol, and/or 1-octen-3-ol levels of one or more genes in the plant with a mutant gene selected from the group consisting of LOX-2 and LOX-3. In some embodiments, methods provided herein comprise decreasing the activity of LOX-2 and LOX-3 genes in the plant with a mutant LOX-2 and LOX-3 genes. In particular, methods of the present disclosure can reduce the level of hexanal, hexanol, 1-octen-3-ol, and/or other volatile compounds (e.g., pentanal, 1-pentanol, 1-penten-3-ol, heptanal, 1-heptanol, octanal 2-octenal (E), 1-octanol, furan, 2-pentyl, nonanal) in a plant or plant part by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, methods of the present disclosure can reduce the level of hexanal, hexanol, 1-octen-3-ol, and/or other volatile compounds (e.g., pentanal, 1-pentanol, 1-penten-3-ol, heptanal, 1-heptanol, octanal 2-octenal (E), 1-octanol, furan, 2-pentyl, nonanal) in a plant or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In specific embodiments, methods of the present disclosure can reduce the level of hexanal in a plant or plant part by at least 70%. In specific embodiments, methods of the present disclosure can reduce the level of 1-hexanol in a plant or plant part by at least 80%.

Also, provided herein are methods of increasing the level of oleic acid in a plant (e.g., Pisum sativum plant) or plant part when compared to a control plant or plant part. In particular, methods comprise decreasing the activity of one or more genes in the plant with a mutant gene selected from the group consisting of LOX-2 and LOX-3. In some embodiments, methods provided herein comprise decreasing the activity of LOX-2 and LOX-3 genes in the plant with a mutant LOX-2 and

LOX-3 genes. The decreased activity in the plant or plant part is by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part expressing one or more WT LOX-2 and WT LOX-3, genes. Additionally, or alternatively, methods of the present disclosure can increase the level of oleic acid in a plant (e.g., Pisum sativum plant) or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant (e.g., Pisum sativum plant) or plant part.

Also provided herein are methods for decreasing the level of linolenic acid in a plant or plant part when compared to a control plant or plant part. In particular, methods of the present disclosure can decrease the level of linolenic acid by decreasing the activity (i.e., function and/or expression) of LOX protein in a plant or plant part by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, methods of the present disclosure can reduce expression and/or function of LOX protein in a plant or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In specific embodiments, the LOX protein is a LOX-2 protein. In some embodiments, the LOX protein is a LOX-3 protein. In specific embodiments, the LOX protein is a LOX-2 and LOX-3 protein.

Reducing the level of hexanal, hexanol, and/or 1-octen-3-ol can improve flavor characteristics of a plant or plant part, and/or can improve flavor characteristics in a plant extract, plant protein, plant concentrate, plant powder, or plant biomass obtained from such plant or plant part. For example, reducing the level of hexanal, hexanol, and/or 1-octen-3-ol can improve flavor aspects described as, e.g., overall aroma, overall flavor impact, beany yellow pea, pyrazine, cereal grain, green grassy green pea, nutty, cardboard, malty, salt, bitter, umami, astringent, or chalky in a plant extract, plant protein, plant concentrate, plant powder, or plant biomass obtained from such plant or plant part. Thus, also provided herein are methods for improving flavor characteristics in plant, plant part, plant extract, plant protein, plant concentrate, plant powder, and/or plant biomass.

Function of a LOX (e.g., LOX-2, LOX-3) protein in a plant or plant part can be reduced by any method known in the art for reduction of protein activity or reduction of gene expression. For example, one or more of the following methods can be used to reduce the total LOX-2 function in a plant.

2.4 Plants with Reduced FAD Function

Provided herein are plants and plant parts with reduced FAD (e.g., FAD2B, FAD3C, FAD3D) function. In particular, plants and plant parts with reduced FAD (e.g., FAD2B, FAD3C, FAD3D) function can have a reduced level of linolenic acid. A plant or plant part described herein (e.g., a plant or plant part containing a mutation in the FAD (e.g., FAD2B, FAD3C, FAD3D) gene) can express a FAD (e.g., FAD2B, FAD3C, FAD3D) protein that comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequences set forth in SEQ ID NOs: 33-35, 43-45, and 53-55. For example, a plant or plant part described herein can have a FAD (e.g., FAD2B, FAD3C, FAD3D) protein that comprises the amino acid sequence of SEQ ID NOs: 33-35, 43-45, and 53-55.

In some embodiments, the gene encoding a FAD2B protein comprises: (a) a nucleic acid sequence having at least 90% sequence identity to the nucleic acid sequence set forth in SEQ ID NO: 36, wherein said nucleic acid sequence encodes a functional FAD2B protein; or (b) the nucleic acid sequence set forth in SEQ ID NO: 36. In some embodiments, the gene encoding a FAD3C protein comprises: (a) a nucleic acid sequence having at least 90% sequence identity to the nucleic acid sequence set forth in SEQ ID NO: 46, wherein said nucleic acid sequence encodes a functional FAD3C protein; or (b) the nucleic acid sequence set forth in SEQ ID NO: 46. In some embodiments, the gene encoding a FAD3D protein comprises: (a) a nucleic acid sequence having at least 90% sequence identity to the nucleic acid sequence set forth in SEQ ID NO: 56, wherein said nucleic acid sequence encodes a functional FAD3D protein; or (b) the nucleic acid sequence set forth in SEQ ID NO: 56.

A plant or plant part described herein (e.g., a plant or plant part containing a mutation in FAD (e.g., FAD2B, FAD3C, FAD3D) gene can express a FAD (e.g., FAD2B, FAD3C, FAD3D) protein that retains FAD function, either fully, or in part. As described in FIG. 9, FAD (e.g., FAD2) catalyzes the insertion of a double bond into 18:1 (oleic acid), forming linoleic acid (18:2). A delta-15 desaturase (FAD3) catalyzes the insertion of a double bond into 18:2, forming linolenic acid (18:3). Linoleic and linolenic acids are the most abundant fatty acids in the lipid fraction of plant membranes and are the major substrates for FADs. Thus, a plant or plant part described herein may express a FAD (e.g., FAD2B, FAD3C, FAD3D) protein that can produce linolenic acid in plants or plant parts by oxidation of oleic acid. In some instances, a plant or plant part described herein may contain a mutation in the FAD (e.g., FAD2B, FAD3C, FAD3D) gene, and thus express a FAD (e.g., FAD2B, FAD3C, FAD3D) protein with reduced function or loss-of-function. Accordingly, such plant or plant part can have reduced level of linolenic acid, as compared to a control plant or plant part, which contains a wild-type (WT) FAD (e.g., FAD2B, FAD3C, FAD3D) gene and expresses a fully functional FAD (e.g., FAD2B, FAD3C, FAD3D) protein. In other instances, a plant or plant part described herein may contain a mutation in the FAD (e.g., FAD2B, FAD3C, FAD3D) gene, and thus express a FAD (e.g., FAD2B, FAD3C, FAD3D) protein with reduced function or loss-of-function. Accordingly, such plant or plant part can have increased level of oleic acid, as compared to a control plant or plant part, which contains a wild-type (WT) FAD (e.g., FAD2B, FAD3C, FAD3D) gene and expresses a fully functional FAD (e.g., FAD2B, FAD3C, FAD3D) protein.

A plant or plant part described herein can contain a mutation in one or more FAD (e.g., FAD2B, FAD3C, FAD3D) gene. In particular, a plant or plant part described herein can contain a FAD (e.g., FAD2B, FAD3C, FAD3D) gene that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NOs: 36-38, 46-48, and 56-58. For example, a plant or plant part described herein can have a FAD gene that comprises the nucleic acid sequence of SEQ ID NOs: 36-38, 46-48, and 56-58. A plant or plant part described herein can comprise 1-6, 2-4, 3-4, 2-5, or 3-5 (e.g., 1, 2, 3, 4, 5, or 6) copies of FAD (e.g., FAD2B, FAD3C, FAD3D) gene. In particular, a plant or plant part described herein can comprise at least 2 genes encoding a FAD (e.g., FAD2B, FAD3C, FAD3D) protein, such as 2 genes that have less than 100% (e.g., less than 100%, 99%, 98%, 97%, 96%, 95%, 94%, 93%, 92%, 91%, 90%, 89%, 88%, 87%, 86%, or 85%) sequence identity.

Described herein are plants or plant parts, in which a gene encoding a FAD (e.g., FAD2B, FAD3C, FAD3D) protein has been mutated (e.g., by one or more insertions, substitutions, or deletions). For example, disclosed herein are plants or plant parts in which a gene encoding the FAD (e.g., FAD2B, FAD3C, FAD3D) protein has been mutated, e.g., by one or more insertions, substitutions, or deletions. In some embodiments, the one or more insertions, substitutions, or deletions are in a region that corresponds to a nucleotide region of a gene encoding the FAD2B protein comprising nucleotides 59 through 66 of SEQ ID NO: 36 or nucleotides 60 through 61 of SEQ ID NO: 36. In some embodiments, said plants or plant parts comprise a nucleic acid molecule comprising SEQ ID NO: 31 or 32. In other embodiments, the one or more insertions, substitutions, or deletions, or part thereof are at least partially in a region that corresponds to a nucleotide region of exon 1 of a gene encoding the FAD2B protein comprising a nucleotide sequence set forth in SEQ ID NO: 29. In some embodiments, the one or more insertions, substitutions, or deletions are in a region that corresponds to a nucleotide region of a gene encoding the FAD3C protein comprising nucleotides 457 through 464 of SEQ ID NO: 46 or nucleotides 416 through 464 of SEQ ID NO: 46. In some embodiments, said plants or plant parts comprise a nucleic acid molecule comprising SEQ ID NO: 41 or 42.insertions, substitutions, or deletions In other embodiments, the one or more insertions, substitutions, or deletions, or part thereof are at least partially in a region that corresponds to a nucleotide region of exon 2 of a gene encoding the FAD3C protein comprising a nucleotide sequence set forth in SEQ ID NO: 39. In some embodiments, the one or more insertions, substitutions, or deletions are in a region that corresponds to a nucleotide region of a gene encoding the FAD3D protein comprising nucleotides 775 through 779 of SEQ ID NO: 56 or nucleotides 745 through 851 of SEQ ID NO: 56. In some embodiments, said plants or plant parts comprise a nucleic acid molecule comprising SEQ ID NO: 51 or 52. In other embodiments, the one or more insertions, substitutions, or deletions, or part thereof are at least partially in a region that corresponds to a nucleotide region of exon 3 of a gene encoding the FAD3D protein comprising a nucleotide sequence set forth in SEQ ID NO: 49.

In some embodiments, the one or more insertions, substitutions, or deletions are in a region that corresponds to a nucleotide region of each of the genes encoding the FAD2B, FAD3C, and FAD3D proteins. In some embodiments, the one or more insertions, substitutions, or deletions are in (i) a region that corresponds to a nucleotide region of the gene(s) encoding the LOX-2 protein and/or LOX-3 protein, and (ii) a region that corresponds to a nucleotide region of the gene(s) encoding the FAD2B, FAD3C, and/or FAD3D proteins. In some embodiments, the one or more insertions, substitutions, or deletions are in a region that corresponds to a nucleotide region of each of the genes encoding the LOX-2 and FAD3C proteins. In some embodiments, the one or more insertions, substitutions, or deletions are in a region that corresponds to a nucleotide region of each of the genes encoding the LOX-2 and FAD2B proteins. In some embodiments, the one or more insertions, substitutions, or deletions are in a region that corresponds to a nucleotide region of each of the genes encoding the LOX-2 and FAD3D proteins. In some embodiments, the one or more insertions, substitutions, or deletions are in a region that corresponds to a nucleotide region of each of the genes encoding the LOX-2 and LOX-3 proteins. In other embodiments, the one or more insertions, substitutions, or deletions, or part thereof are at least partially in a region that corresponds to a nucleotide region of exon 1, exon 2, and exon 3 of genes encoding FAD2B, FAD3C, and FAD3D proteins, respectively. In some embodiments, the one or more insertions, substitutions, or deletions are in a region that corresponds to a nucleotide region of each of the genes encoding the LOX-2, LOX-3, FAD2B, FAD3C, and FAD3D proteins. In other embodiments, the one or more insertions, substitutions, or deletions, or part thereof are at least partially in a region that corresponds to a nucleotide region of exon 4, exon 4, exon 1, exon 2, and exon 3 of genes encoding LOX-2, LOX-3, FAD2B, FAD3C, and FAD3D proteins, respectively. In many embodiments described herein, the deletion is an out-of-frame deletion. In other embodiments described herein, the deletion is an in-frame deletion.

Also provided herein are plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant protein, plant concentrate (e.g., whole plant concentrate or plant part concentrate), plant powder (e.g., formulated powder, such as formulated plant part powder (e.g., seed flour)), and plant biomass (e.g., dried biomass, such as crushed and/or powdered biomass) obtained from plants with such mutation in a FAD (e.g., FAD2B, FAD3C, FAD3D) gene. Also provided herein are seeds, such as a representative sample of seeds, from a plant of the present disclosure. In some embodiments, the plants or plant parts comprise a FAD2B protein that has been mutated (e.g., by one or more insertions, substitutions, or deletions). In some embodiments described herein, the plants or plant parts comprise an altered FAD2B protein that has been further mutated (e.g., by one or more insertions, substitutions, or deletions). In many embodiments described herein, the plants or plant parts comprise a FAD2B protein that has an amino acid sequence having at least 90% sequence identity to the amino acid sequence set forth in SEQ ID NOs: 33-35. In some embodiments, the plants or plant parts comprise a FAD2B protein that has an amino acid sequence set forth in SEQ ID NOs: 33-35. In some other embodiments, the plants or plant parts comprise a FAD3C protein that has been mutated (e.g., by one or more insertions, substitutions, or deletions). In some embodiments described herein, the plants or plant parts comprise an altered FAD3C protein that has been further mutated (e.g., by one or more insertions, substitutions, or deletions). In many embodiments described herein, the plants or plant parts comprise a FAD3C protein that has an amino acid sequence having at least 90% sequence identity to the amino acid sequence set forth in SEQ ID NOs: 43-45. In some embodiments, the plants or plant parts comprise a FAD3C protein that has an amino acid sequence set forth in SEQ ID NOs: 43-45. In some other embodiments, the plants or plant parts comprise a FAD3D protein that has been mutated (e.g., by one or more insertions, substitutions, or deletions). In some embodiments described herein, the plants or plant parts comprise an altered FAD3D protein that has been further mutated (e.g., by one or more insertions, substitutions, or deletions). In many embodiments described herein, the plants or plant parts comprise a FAD3D protein that has an amino acid sequence having at least 90% sequence identity to the amino acid sequence set forth in SEQ ID NOs: 53-55. In some embodiments, the plants or plant parts comprise a FAD3D protein that has an amino acid sequence set forth in SEQ ID NOs: 53-55.

In certain instances, mutations in any FAD (e.g., FAD2B, FAD3C, FAD3D) gene in a plant, plant part, or protein composition obtained from plant or plant part can be identified by a diagnostic method described herein. Such diagnostic methods may comprise use of primers for detecting mutation in FAD (e.g., FAD2B, FAD3C, FAD3D) gene. For example, forward primer 005 (SEQ ID NO: 61) and reverse primer 006 (SEQ ID NO: 62) can be used for detection of mutation in FAD2B gene. The forward primer 012 (SEQ ID NO: 63) and reverse primer 020 (SEQ ID NO: 64) can be used for detection of mutation in FAD3C gene. The forward primer 021 (SEQ ID NO: 65) and reverse primer 030 (SEQ ID NO: 66) can be used for detection of mutation in FAD3D gene. In certain instances, a kit comprising a set of primers can be used for detecting mutation of FAD (e.g., FAD2B, FAD3C, FAD3D) gene in plants, plant parts, or protein composition obtained from plants or plant parts. For example, a kit comprising forward primer 005 (SEQ ID NO: 61) and reverse primer 006 (SEQ ID NO: 62) can be used for detection of mutation in FAD2B gene in plants, plant parts, or protein composition obtained from plants or plant parts. In some embodiments, a kit comprising forward primer 012 (SEQ ID NO: 63) and reverse primer 020 (SEQ ID NO: 64) can be used for detection of mutation in FAD3C gene in plants, plant parts, or protein composition obtained from plants or plant parts. In some other embodiments, a kit comprising forward primer 021 (SEQ ID NO: 65) and reverse primer 030 (SEQ ID NO: 66) can be used for detection of mutation in FAD3D gene in plants, plant parts, or protein composition obtained from plants or plant parts.

In many embodiments described herein, the plants or plant parts comprise Pisum sativum plants or plant parts.

(i) Plants with Reduced Expression of Full-Length FAD Protein

A plant or plant part of the present disclosure can have reduced expression of a FAD (e.g., FAD2B, FAD3C, FAD3D) protein, as compared to a control plant or plant part, such as a plant or plant part that contains an unmutated and/or WT FAD gene. In a particular embodiment, a plant (e.g., Pisum sativum) or plant part of the present disclosure can have reduced expression of a LOX-2 and FAD3C protein, as compared to a control plant or plant part, such as a plant or plant part that contains an unmutated and/or WT LOX-2 and FAD3C gene. In particular, a plant or plant part that contains a mutated FAD gene can have reduced expression of a full length FAD (e.g., FAD2B, FAD3C, FAD3D) protein, as compared to a control plant or plant part. For example, a plant or plant part that contains a mutated FAD2B gene can have reduced expression of full length FAD2B protein, as compared to a control plant or plant part. A plant or plant part that contains a mutated FAD3C gene can have reduced expression of full length FAD3C protein, as compared to a control plant or plant part. A plant or plant part that contains a mutated FAD3D gene can have reduced expression of full length FAD3D protein, as compared to a control plant or plant part. A control plant or plant part can be a plant or plant part that has a full-length or wild-type FAD (e.g., FAD2B,

FAD3C, FAD3D) gene. For example, a control plant or plant part can be a plant or plant part before a FAD (e.g., FAD2B, FAD3C, FAD3D) gene in the plant or plant part is mutated. Thus, a control plant or plant part may express a WT FAD (e.g., one or more of FAD2B, FAD3C, FAD3D) gene. A control plant of the present disclosure may be grown under the same environmental conditions (e.g., same or similar temperature, humidity, air quality, soil quality, water quality, and/or pH conditions) as a plant that contains a mutated FAD (e.g., FAD2B, FAD3C, FAD3D) gene. A plant or plant part containing a mutated FAD gene can have reduced expression of one or more FAD (e.g., one or more of FAD2B, FAD3C, FAD3D) proteins, as compared to a control plant or plant part, when the plant or plant part with the mutated FAD gene is grown under the same environmental conditions as the control plant or plant part. In some embodiments, expression of one or more FAD (e.g., FAD2B, FAD3C, FAD3D) proteins in a plant or plant part with a mutated FAD gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, expression of FAD (e.g., FAD2B, FAD3C, FAD3D) protein in a plant or plant part, which contains a mutated FAD gene, can be reduced by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In specific embodiments, the FAD protein is a FAD2B protein. In other embodiments, the FAD protein is a FAD3C protein. In yet another embodiment, the FAD protein is a FAD3D protein.

The plant parts, plant extracts, plant protein, plant concentrate, plant powder, and/or plant biomass, which is obtained from plants containing one or more FAD genes mutated, can have reduced expression of one or more FAD (e.g., FAD2B, FAD3C, FAD3D) proteins or reduced activity of one or more FADs (e.g., FAD2B, FAD3C, FAD3D), as compared to plant parts, plant extracts, protein, plant concentrate, plant powder, and/or plant biomass obtained from a control plant. In particular, plant part, plant extract, plant protein, plant concentrate, plant powder, and/or plant biomass obtained from plants with a mutated FAD gene can have reduced expression of a full length FAD (e.g., FAD2B, FAD3C, FAD3D) protein, as compared to plant part, plant extract, plant protein, plant concentrate, plant powder, and/or plant biomass obtained from a control plant. For example, Pisum sativum plant part, plant extract, plant protein, plant concentrate, plant powder, and/or plant biomass obtained from a Pisum sativum plant with a mutated FAD2B gene can have reduced expression of full length FAD2B protein, as compared to Pisum sativum plant part, plant extract, plant protein, plant concentrate, plant powder, and/or plant biomass obtained from a control plant. The plant part, plant extract, plant protein, plant concentrate, plant powder, and/or plant biomass obtained from a plant with a mutated FAD2B gene can have reduced activity of FAD2B protein, as compared to plant part, plant extract, plant protein, plant concentrate, plant powder, and/or plant biomass obtained from a control plant, when the plant with the mutated FAD2B gene is grown under the same environmental conditions as the control plant. In some embodiments, a plant part, plant extract, plant protein, plant concentrate, plant powder, and/or plant biomass obtained from a plant with a mutated FAD3C gene can have reduced expression of full length FAD3C protein, as compared to plant part, plant extract, plant protein, plant concentrate, plant powder, and/or plant biomass obtained from a control plant. The plant part, plant extract, plant protein, plant concentrate, plant powder, and/or plant biomass obtained from a plant with a mutated FAD3C gene can have reduced activity of FAD3C protein, as compared to plant part, plant extract, plant protein, plant concentrate, plant powder, and/or plant biomass obtained from a control plant, when the plant with the mutated FAD3C gene is grown under the same environmental conditions as the control plant. In some other embodiment, a plant part, plant extract, plant protein, plant concentrate, plant powder, and/or plant biomass obtained from a plant with a mutated FAD3D gene can have reduced expression of full length FAD3D protein, as compared to plant part, plant extract, plant protein, plant concentrate, plant powder, and/or plant biomass obtained from a control plant. The plant part, plant extract, plant protein, plant concentrate, plant powder, and/or plant biomass obtained from a plant with a mutated FAD3D gene can have reduced activity of FAD3D protein, as compared to plant part, plant extract, plant protein, plant concentrate, plant powder, and/or plant biomass obtained from a control plant, when the plant with the mutated FAD3D gene is grown under the same environmental conditions as the control plant. In many embodiments described herein, the plants or plant parts comprise Pisum sativum plants or plant parts.

In some embodiments, expression of FAD (e.g., FAD2B, FAD3C, FAD3D) protein in plant part, plant extract, plant protein, plant concentrate, plant powder, and/or plant biomass obtained from a plant with a mutated FAD gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to the plant part, plant extract, plant protein, plant concentrate, plant powder, and/or plant biomass obtained from a control plant. Additionally, or alternatively, expression of FAD (e.g., FAD2B, FAD3C, FAD3D) protein in plant part, plant extract, plant protein, plant concentrate, plant powder, and/or plant biomass obtained from a plant with a mutated FAD gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to plant part, plant extract, plant protein, plant concentrate, plant powder, and/or plant biomass obtained from a control plant. In specific embodiments, the FAD protein is a FAD2B protein. In some embodiments, the FAD protein is a FAD3C protein. In other embodiments, the FAD protein is a FAD3D protein. In some embodiments, expression of FAD3C and LOX-2 proteins in plant part, plant extract, plant protein, plant concentrate, plant powder, and/or plant biomass obtained from a plant with a mutated FAD3C and LOX-2 genes can each be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to the plant part, plant extract, plant protein, plant concentrate, plant powder, and/or plant biomass obtained from a control plant comprising WT FAD3C and LOX-2 genes.

Expression of a FAD (e.g., FAD2B, FAD3C, FAD3D) protein, such as a full length FAD2B protein, in a plant, plant part, plant extract, plant protein, plant concentrate, plant powder, and/or plant biomass can be determined by one or more standard methods known in the art. In some embodiments, expression of a FAD protein can be determined by western blot analysis of a protein sample obtained from a plant, plant part, plant extract, plant protein, plant concentrate, plant powder, and/or plant biomass by using an antibody directed to the FAD (e.g., FAD2B, FAD3C, FAD3D) protein. For example, expression of a full length FAD protein can be determined by western blot analysis of a protein sample obtained from a plant, plant part, plant extract, plant protein, plant concentrate, plant powder, and/or plant biomass by using an antibody directed to the full length FAD (e.g., FAD2B, FAD3C, FAD3D) protein. Details of such procedure has been outlined in the Examples section of the present disclosure.

(ii) Plants with Loss-of-Function or Reduced Function in FAD Protein

A plant or plant that contains a mutated FAD gene can have loss-of-function or reduced function in the encoded FAD (e.g., FAD2B, FAD3C, FAD3D) protein, as compared to a control plant or plant part. For example, a Pisum sativum plant or plant part that contains a mutated FAD2B gene can have loss-of-function or reduced function (i.e., reduced FAD2B activity) in the encoded FAD2B protein, as compared to a control plant or plant part. In some embodiments, a plant or plant part that contains a mutated FAD3C gene can have loss-of-function or reduced function (i.e., reduced FAD3C activity) in the encoded FAD3C protein, as compared to a control plant or plant part. In other embodiments, a plant or plant part that contains a mutated FAD3D gene can have loss-of-function or reduced function (i.e., reduced FAD3D activity) in the encoded FAD3D protein, as compared to a control plant or plant part. A control plant or plant part can be a plant or plant part that does not contain a mutation in the FAD gene and/or contain a WT FAD gene. For example, a control plant or plant part can be a plant or plant part before a FAD gene in the plant or plant part is mutated.

Thus, a control plant or plant part may express WT FAD (e.g., FAD2B, FAD3C, FAD3D) gene. A control plant of the present disclosure may be grown under the same environmental conditions (e.g., same or similar temperature, humidity, air quality, soil quality, water quality, and/or pH conditions) as a plant that contains the mutated FAD gene. A plant or plant part that contains a mutated FAD gene can have loss-of-function or reduced function in the encoded FAD protein, as compared to a control plant or plant part, when the plant or plant part with a mutated FAD gene is grown under the same environmental conditions as the control plant or plant part. In some embodiments, the activity of one or more FAD (e.g., FAD2B, FAD3C, FAD3D) in a plant or plant part with a mutated FAD gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In some embodiments, activity of FAD3C and LOX-2 in a plant or plant part with a mutated FAD3C and LOX-2 genes can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part comprising WT FAD3C and LOX-2 proteins. Additionally, or alternatively, the activity of one or more FAD (e.g., FAD2B, FAD3C, FAD3D) in a plant or plant part with a mutated FAD gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part.

Also, plant part, plant extract, plant protein, plant concentrate, plant powder, and/or plant biomass obtained from plants with a mutated FAD gene can have loss-of-function or reduced function of encoded FAD (e.g., FAD2B, FAD3C, FAD3D) protein, as compared to plant part, plant extract, plant protein, plant concentrate, plant powder, and/or plant biomass obtained from a control plant. For example, plant part, plant extract, plant protein, plant concentrate, plant powder, and/or plant biomass obtained from a plant with a mutated FAD2B gene can have loss-of-function or reduced function of encoded FAD2B protein, as compared to plant part, plant extract, plant protein, plant concentrate, plant powder, and/or plant biomass obtained from a control plant. Plant part, plant extract, plant protein, plant concentrate, plant powder, and/or plant biomass obtained from a plant with a mutated FAD gene can have loss-of-function or reduced function in the encoded FAD protein, as compared to plant part, plant extract, plant protein, plant concentrate, plant powder, and/or plant biomass obtained from a control plant, when the plant with a mutated FAD gene is grown under the same environmental conditions as the control plant. In some embodiments, function of encoded FAD protein in plant part, plant extract, plant protein, plant concentrate, plant powder, and/or plant biomass obtained from a plant with a mutated FAD gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to plant part, plant extract, plant protein, plant concentrate, plant powder, and/or plant biomass obtained from a control plant. In some embodiments, function of encoded FAD3C and LOX-2 proteins in plant part, plant extract, plant protein, plant concentrate, plant powder, and/or plant biomass obtained from a plant with a mutated FAD3C and LOX-2 genes can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part comprising WT FAD3C and LOX-2 proteins. Additionally, or alternatively, function of encoded FAD protein in plant part, plant extract, plant protein, plant concentrate, plant powder, and/or plant biomass obtained from a plant with a mutated FAD gene can be reduced by at least 10%, 15%, 20%, 25%, 30%, 35%, 40%, 45%, 50%, 55%, 60%, 65%, 70%, 75%, 80%, 85%, 90%, 95%, or 100%, as compared to plant part, plant extract, plant protein, plant concentrate, plant powder, and/or plant biomass obtained from a control plant. In specific embodiments, the FAD protein is a FAD2B protein. In some embodiments, the FAD protein is a FAD3C protein. In other embodiments, the FAD protein is a FAD3D protein. In many embodiments described herein, the plants or plant parts comprise Pisum sativum plants or plant parts.

Function of encoded FAD (e.g., FAD2B, FAD3C, FAD3D) protein in a plant, plant part, plant extract, plant protein, plant concentrate, plant powder, and/or plant biomass can be determined by one or more standard methods known in the art. In some embodiments, function of encoded FAD protein can be determined by assessing enzyme activity of FAD in a protein sample obtained from a plant, plant part, plant extract, plant protein, plant concentrate, plant powder, and/or plant biomass. FAD enzyme activity can be determined by measuring fluorescence signal generated by the reaction of a fluorescent probe with oxidized fatty acid, an intermediate that is produced when FAD protein acts on a substrate of FAD. For example, enzyme activities of FAD2B, FAD3C and/or FAD3D proteins can be determined by using linolenic acid as a substrate. Details of such procedure has been outlined in the Examples section of the present disclosure.

(iii) Plants with Reduced Level of Volatile Compounds

Gene inactivation approaches such as post transcriptional gene silencing (PTGS) have been successfully applied to inactivate fatty acid biosynthetic genes and develop nutritionally improved plant oils in oilseed crops. For example, soybean lines with 80% oleic acid in their seed oil were created by co-suppression of the FAD2 encoded microsomal Δ12-desaturase (Kinney, 1996). Mutating a gene encoding a FAD (e.g., FAD2B, FAD3C, FAD3D) protein can improve flavor characteristics of a plant or plant part by reducing the level of linolenic acid in such plant or plant part. For example, mutating a gene encoding a FAD (e.g., FAD2B, FAD3C, FAD3D) protein can improve flavor aspects of a plant or plant part described as, e.g., overall aroma, overall flavor impact, beany yellow pea, pyrazine, cereal grain, green grassy green pea, nutty, cardboard, malty, salt, bitter, umami, astringent. Thus, a plant or plant part with a mutated FAD gene can have improved flavor characteristics compared to a control plant or plant part.

Flavor characteristics of various plants (e.g., Pisum plant), plant part, or protein composition obtained from plant or plant part may refer to taste or aroma of the plant, plant part, or protein composition. Linoleic and linolenic acids are PUFAs that are essential for health and nutrition. Despite health benefits of PUFAs, they make the oil, protein concentrates obtained from various species of plant (e.g., Pisum plant) or plant part, more vulnerable to rancidity, decrease its flavor, and shorten its shelf life. The oxidative stability and nutritional value of the oil and/or protein concentrates are dependent upon the fatty acid content therein, especially of oleic and linoleic acids. Oleic acid was found to have higher oxidative stability than linoleic acid, resulting in the extension of its shelf life. Therefore, there is a high demand for premium quality oil and/or protein concentrates obtained from various species of plant (e.g., Pisum plant) or plant part, that are rich in monounsaturated fatty acids and poor in PUFAs. Thus, decreasing the linolenic acid content and/or increasing the oleic acid content in oil and/or protein concentrates obtained various species of plant (e.g., Pisum plant) or plant part is of significant commercial importance. The desaturation of fatty acids by FAD desaturases is one of the important biochemical processes that define the quality and quantity of PUFA content in oil and/or protein concentrates obtained various species of plant (e.g., Pisum plant) or plant part

Volatile compounds that contribute to flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky) of various species of plant (e.g., Pisum plant), plant part, or protein composition obtained from plant or plant part can be quantified by using Gas Chromatography-Mass Spectroscopy (GC-MS), a lab-based technique which helps to separate and identify compounds in their gaseous forms based on their masses. In certain instances, to correlate these instrumental measurements to consumer perception, two major methods of sensory evaluation are used: consumer testing and descriptive analysis. As described in the previous sections, consumer testing includes subjective data about the preferences of a large group of untrained tasters (usually more than 100 panelists), while descriptive analysis includes questionnaires for a panel of 8-12 trained tasters who are able to rate specific attributes related to flavor or aroma. Methods for determining flavor characteristic of plants and plant parts is described in the art, e.g., by Barrett et al. (Critical Reviews in Food Science and Nutrition, 50(5): 369-389 (2010)) and Hallowell et al. (Chem Senses, 41(3):249-259 (2016)). In certain instances, flavor characteristics of plant, plant part, or protein composition obtained from plant or plant part can be determined by a flavor panel experiment. Such flavor panel experiment may use instrumental measurements, sensory testing, or a combination thereof. Plant, plant part, or protein composition that scores higher (as compared to a suitable control) in such flavor panel experiments can be considered to have improved flavor characteristics. For example, in a flavor panel experiment, a plant or plant part containing mutation in FAD2B gene can score higher compared to a control plant or plant part (e.g., plant or plant part that does not contain mutation in FAD2B gene), and thus can be considered to have improved flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky) compared to the control plant or plant part.

A control plant or plant part can be a plant or plant part that does not contain a mutated FAD gene. For example, a control plant or plant part can be a plant or plant part before FAD gene in the plant or plant part is mutated. Thus, a control plant or plant part may express WT FAD (e.g., FAD2B, FAD3C, FAD3D) gene. A control plant of the present disclosure may be grown under the same environmental conditions (e.g., same or similar temperature, humidity, air quality, soil quality, water quality, and/or pH conditions) as a plant with a mutated FAD gene. A plant or plant part with a mutated FAD gene can have improved flavor characteristics, as compared to a control plant or plant part, when the plant or plant part with a mutated FAD gene is grown under the same environmental conditions as the control plant or plant part. Improved flavor characteristics of a plant or plant part with a mutated FAD gene can result from reduced level of linolenic acid and/or increased levels of oleic acid in such plants or plant parts.

The plant parts, plant extracts, plant protein, plant concentrate, plant powder, and/or plant biomass obtained from a plant with a mutated FAD (e.g., FAD2B, FAD3C, FAD3D) gene can have improved flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky) compared to plant part, plant extract, plant protein, plant concentrate, plant powder, and/or plant biomass obtained from a control plant. Plant part, plant extract, plant protein, plant concentrate, plant powder, and/or plant biomass obtained from a plant with a mutated FAD gene can have improved flavor characteristics, as compared to plant part, plant extract, plant protein, plant concentrate, plant powder, and/or plant biomass obtained from a control plant, when the plant with a mutated FAD gene is grown under the same environmental conditions as the control plant. Improved flavor characteristics of plant part, plant extract, plant protein, plant concentrate, plant powder, and/or plant biomass obtained from a plant with a mutated FAD gene can result from reduced level of one or more volatile compounds and/or modified level of one or more fatty acids in such plant part, plant extract, plant protein, plant concentrate, plant powder, and/or plant biomass.

Plants or plant parts having a mutated LOX gene and/or FAD gene (e.g., LOX-2, LOX-3, FAD2B, FAD3C, FAD3D) can have characteristics provided herein, e.g., reduced level or activity of the LOX gene or FAD gene, reduced level of hexanal and/or 1-hexanol, improved flavor characteristics, and have no significant decrease (e.g., no statistically significant decrease, no more than 20% decrease) in yield or total protein content as compared to a control plant or plant part (e.g., wild type, having no mutation). Plants or plant parts having a mutated LOX gene and/or FAD gene can have yields and/or total protein content of at least 80% (e.g., 80%, 85%, 90%, 95%, 99%, 100%, or more) as compared to a control plant or plant part. Yield can be measured and expressed by any means known in the art. In specific embodiments, yield is measured by seed weight (e.g., seed dry weight) or seed volume (e.g., seed dry volume) in a given harvest area. Protein content can be measured and expressed by any means known in the art, for example by protein extraction and quantitation (e.g., BCA protein assay, Lowry protein assay, Bradford protein assay), spectroscopy, near-infrared reflectance (NIR) (e.g., analyzing 700-2500 nm), and nuclear magnetic resonance spectrometry (NMR).

(iv) Plants and Plant Products with Modified Levels of Linolenic Acid, Linoleic Acid, Oleic Acid, and/or Palmitic Acid

A plant or plant part with a mutated FAD gene and/or LOX gene (e.g., a mutated FAD2B gene, a mutated FAD3C gene, a mutated FAD3D gene, a mutated LOX-2 gene, a mutated LOX-3 gene, mutated FAD2B and FAD3C genes, mutated FAD2B and FAD3C genes, mutated FAD2B, FAD3C, and FAD3D genes, mutated LOX-2 and LOX-3 genes, mutated LOX-2 and FAD2B genes, mutated LOX-2 and FAD3C genes, mutated LOX-2 and FAD3D genes, mutated LOX-2, FAD2B, and FAD3C genes, mutated LOX-2, FAD2B, and FAD3D genes, mutated LOX-2, FAD3C, and FAD3D genes, mutated LOX-2, FAD2B, FAD3C, and FAD3D genes, mutated LOX-3 and FAD2B genes, mutated LOX-3 and FAD3C genes, mutated LOX-3 and FAD3D genes, mutated LOX-3, FAD2B, and FAD3C genes, mutated LOX-3, FAD2B, and FAD3D genes, mutated LOX-3, FAD3C, and FAD3D genes, mutated LOX-3, FAD2B, FAD3C, and FAD3D genes, mutated LOX-2, LOX-3, and FAD2B genes, mutated LOX-2, LOX-3, and FAD3C genes, mutated LOX-2, LOX-3, and FAD3D genes, mutated LOX-2, LOX-3, FAD2B, and FAD3C genes, mutated LOX-2, LOX-3, FAD2B, and FAD3D genes, mutated LOX-2, LOX-3, FAD3C, and FAD3D genes, mutated LOX-2, LOX-3, FAD2B, FAD3C, and FAD3D genes) can have reduced level of linolenic acid; reduced level of linoleic acid; increased levels of oleic acid; increased level of oleic acid and reduced level of linoleic acid; increased level of oleic acid and reduced level of linolenic acid; reduced level of linoleic acid plus linolenic acid; increased level of oleic acid, and reduced level of linoleic acid plus linolenic acid; increased level of monounsaturated fat; reduced level of polyunsaturated fat; or increased level of monounsaturated fatty acid and reduced level of polyunsaturated fatty acid, as compared to a control plant or plant part, when the plant or plant part with a mutated FAD gene is grown under the same environmental conditions as the control plant or plant part. In some embodiments, level of linolenic acid in a plant or plant part with a mutated FAD and/or LOX gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part.

In some embodiments, level of linoleic acid in a plant or plant part, or plant extract, plant protein, plant concentrate, plant powder, or plant biomass obtained from a plant with a mutated FAD and/or LOX gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part.

In some embodiments, level of oleic acid in a plant or plant part, or plant extract, plant protein, plant concentrate, plant powder, or plant biomass obtained from a plant with a mutated FAD and/or LOX gene can be increased by about 1-100%, 4-100%, 5-100%, 10-100%, 20-100%, 30-100%, 40-100%, 50-100%, 60-100%, 70-100%, 80-100%, 20-90%, 30-90%, 40-90%, 50-90%, 60-90%, 70-90%, 100-150%, 200-150%, 300-150%, 400-150%, 500-150%, 600-150%, 700-150%, 800-150%, 200-200%, 300-200%, 400-200%, 500-200%, 600-200%, 700-200%, or more than 200% (e.g., by about 1-4%, 4-10%, 10-20%, 20-30%, 30-40%, 40-50%, 50-60%, 60-70%, 70-80%, 80-90%, 90-100%, 100-150%, 150-200%, or more than 200%), e.g., by about 1%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, or 200% as compared to a control plant or plant part.

A plant or plant part with a mutated FAD gene and/or LOX gene can further have a mutation (e.g., one or more insertions, substitutions, or deletions) in a β-ketoacyl-ACP synthase (β-ketoacyl-acyl-carrier-protein synthase; referred to as “KAS”) gene. KAS is an enzyme involved in control of chain length of an acyl group in the fatty acid synthesis pathway. In the plants, four types (KAS I, KAS II, KAS III, and KAS IV) are known to exist. KAS III functions in a stage of starting a chain length elongation reaction to elongate the acetyl-ACP having 2 carbon atoms to the acyl-ACP having 4 carbon atoms. In the subsequent elongation reaction, KAS II (EC 2.3.1.41) catalyzes the elongation of palmitoyl-ACT (having 16 carbon atoms) to stearoyl-ACP (having 18 carbon atoms) and thereby the synthesis of stearic acid from palmitic acid. In some embodiments, a plant or plant part having a mutated KAS II has reduced level and/or activity of KAS II, and has increased level of palmitic acid. In some embodiments, level of palmitic acid in a plant or plant part, or plant extract, plant protein, plant concentrate, plant powder, or plant biomass obtained from a plant with a mutated (having reduced level or gene can be increased by about 1-100%, 5-100%, 10-100%, 20-100%, 30-100%, 40-100%, 50-100%, 60-100%, 70-100%, 80-100%, 20-90%, 30-90%, 40-90%, 50-90%, 60-90%, 70-90%, 100-150%, 200-150%, 300-150%, 400-150%, 500-150%, 600-150%, 700-150%, 800-150%, 200-200%, 300-200%, 400-200%, 500-200%, 600-200%, 700-200%, or more than 200% (e.g., by about 1-5%, 5-10%, 10-20%, 20-30%, 30-40%, 40-50%, 50-60%, 60-70%, 70-80%, 80-90%, 90-100%, 100-150%, 150-200%, or more than 200%), e.g., by about 1%, 5%, 10%, 15%, 20%, 25%, 30%, 35%, 40%, 45%, 50%, 55%, 60%, 65%, 70%, 75%, 80%, 85%, 90%, 95%, 100%, 150%, or 200% as compared to a control plant or plant part. A plant or plant part with a mutated FAD gene and/or LOX gene, and mutated KAS II gene can have reduced level of linolenic acid; reduced level of linoleic acid; increased levels of oleic acid; increased level of palmitic acid; increased level of oleic acid and reduced level of linoleic acid; increased level of oleic acid and reduced level of linolenic acid; reduced level of linoleic acid plus linolenic acid; increased level of oleic acid, and reduced level of linoleic acid plus linolenic acid; increased level of palmitic acid and reduced level of linoleic acid; increased level of palmitic acid and reduced level of linolenic acid; increased level of palmitic acid, and reduced level of linoleic acid plus low linolenic acid; increased level of palmitic acid, increased level of oleic acid, and reduced level of linoleic acid; increased level of palmitic acid, increased level of oleic acid, and reduced level of linolenic acid; increased level of palmitic acid, increased level of oleic acid, and reduced level of linoleic acid plus linolenic acid; increased level of saturated fat; increased level of monounsaturated fat; reduced level of polyunsaturated fat; increased level of saturated fatty acid and increased level of monounsaturated fat; increased level of saturated fatty acid and reduced level of polyunsaturated fat; increased level of monounsaturated fatty acid and reduced level of polyunsaturated fat; or increased level of saturated fatty acid, increased level of monounsaturated fatty acid, and reduced level of polyunsaturated fatty acid, as compared to a control plant (e.g., without a mutation) grown under the same environmental conditions as the plant with mutation.

The amount or level of palmitic acid, linolenic acid, linoleic acid, and/or oleic acid in a plant (e.g., Pisum sativum), plant part, plant extract, plant protein, plant concentrate, plant powder, and/or plant biomass can be determined by one or more standard methods known in the art. In some embodiments, amount or level of linolenic acid and/or oleic acid in a Pisum sativum plant, plant part, plant extract, plant protein, plant concentrate, plant powder, and/or plant biomass is determined by Solid-Phase Micro-Extraction (SPME) and Gas Chromatography (GC). Details of such procedure has been outlined in the Examples section of the present disclosure.

Also provided herein are plant products produced from plants or plant parts provided herein (e.g., having decreased LOX and/or FAD activity, having mutated LOX, FAD2, and/or FAD3 gene, having decreased LOX, FAD, and/or KAS II activity, having mutated LOX, FAD2, FAD3, and/or KAS II gene). “Plant products” can include any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein. Plant parts and plant products provided herein can be intended for human or animal consumption.

The plant products provided herein can comprise reduced levels of hexanal, hexanol, or linolenic acid, and/or an increased level of oleic acid. In specific embodiments, provided herein are a protein composition (e.g., yellow pea protein concentrate) and oil, such as a protein composition or oil obtained (e.g., extracted or isolated) from a Pisum species plant that contains mutated LOX, FAD, and/or KAS II gene. In a particular embodiment, provided herein is a protein composition or oil obtained from a pea plant (Pisum sativum) that contains mutated LOX (e.g., LOX-2, LOX-3) and/or FAD (e.g., FAD2B, FAD3C, FAD3D) gene. In another specific embodiment, provided herein is a protein composition or oil obtained from a pea plant (Pisum sativum) that contains mutated LOX (e.g., LOX-2, LOX-3) and/or FAD (e.g., FAD2B, FAD3C, FAD3D) gene, and a mutated KAS II gene.

The protein composition can comprise multiple proteins as a result of the extraction or isolation process. In specific embodiments, the protein composition can further comprise stabilizers, excipients, drying agents, desiccating agents, anti-caking agents, or any other ingredient to make the protein fit for the intended purpose. The protein composition can be a solid, liquid, gel, or aerosol and can be formulated as a powder. The protein composition can be extracted in a powder form from a plant and can be processed and produced in different ways, such as: (i) as an isolate—through the process of wet fractionation, which has the highest protein concentration; (ii) as a concentrate—through the process of dry fractionation, which are lower in protein concentration; and/or (iii) in textured form—when it is used in food products as a substitute for other products, such as meat substitution (e.g. a “meat” patty).

Plant parts (e.g., seeds) and plant products (e.g., plant biomass, seed compositions, protein compositions, food and/or beverage products) produced by the methods provided herein can be meant for consumption by agricultural animals or for use as feed in an agriculture or aquaculture system. In specific embodiments, plant parts and plant products produced according to the methods provided herein include animal feed (e.g., roughages—forage, hay, silage; concentrates—cereal grains, soybean cake) intended for consumption by bovine, porcine, poultry, lambs, goats, or any other agricultural animal. In some embodiments, plant parts and plant products produced according to the methods include aquaculture feed for any type of fish or aquatic animal in a farmed or wild environment including, without limitation, trout, carp, catfish, salmon, tilapia, crab, lobster, shrimp, oysters, clams, mussels, and scallops.

A protein composition (e.g., yellow pea protein concentrate) or oil obtained (i.e., extracted or isolated) from a Pisum sativum plant with a mutated LOX (e.g., LOX-2, LOX-3) and/or FAD (e.g., FAD2B, FAD3C, FAD3D) gene can have improved flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky) compared to protein composition or oil obtained from a control plant. Protein composition or oil obtained from a plant with a mutated LOX (e.g., LOX-2, LOX-3) and/or FAD (e.g., FAD2B, FAD3C, FAD3D) gene can have improved flavor characteristics, as compared to protein composition or oil obtained from a control plant, when the plant with a mutated LOX (e.g., LOX-2, LOX-3) and/or FAD (e.g., FAD2B, FAD3C, FAD3D) gene is grown under the same environmental conditions as the control plant. Improved flavor characteristics of protein compositions or oil obtained from a plant with a mutated LOX (e.g., LOX-2, LOX-3) and/or FAD (e.g., FAD2B, FAD3C, FAD3D) gene can result from reduced level of linolenic acid and/or increased level of oleic acid in such protein composition or oil. Protein composition or oil obtained from a Pisum sativum plant with a mutated FAD2B, FAD3C, and/or FAD3D gene can have reduced level of linolenic acid and/or increased level of oleic acid, as compared to protein composition or oil obtained from a control plant, when the plant with mutated FAD2B, FAD3C, and/or FAD3D gene is grown under the same environmental conditions as the control plant.

Provided herein are plant products (e.g., plant oil, protein compositions) produced from the plant or plant part provided herein having reduced activity of LOX and/or FAD, or from a plant or plant part having reduced activity of LOX and/or FAD produced by the method provided herein, comprising: high oleic acid content; low linoleic acid content; low linolenic acid content; high oleic acid and low linoleic acid content; high oleic acid and low linolenic acid content; low linoleic acid and low linolenic acid content; high oleic acid, low linoleic acid, and low linolenic acid content; high monounsaturated fatty acid content; low polyunsaturated fatty acid content; or high monounsaturated fatty acid and low polyunsaturated fatty acid content, relative to a product produced from a control plant or plant part. In some embodiments, the plant product (e.g., oil, protein composition) comprises high monounsaturated fatty acid to polyunsaturated fatty acid composition relative to a control product. Further provided herein are plant products (e.g., plant oil, protein compositions) produced from the plant or plant part provided herein having reduced activity of LOX and/or FAD and reduced activity of KAS II, or from a plant or plant part having reduced activity of LOX and/or FAD and reduced activity of KAS II produced by the method provided herein, comprising: high palmitic acid content; high palmitic acid and high oleic acid content; high palmitic acid and low linoleic acid content; high palmitic acid and low linolenic acid content; high palmitic acid, high oleic acid, and low linoleic acid content; high palmitic acid, high oleic acid, and low linolenic acid content; high palmitic acid, low linoleic acid, and low linolenic acid content; high palmitic acid, high oleic acid, low linoleic acid, and low linolenic acid content, high unsaturated fatty acid content; high unsaturated fatty acid and high monounsaturated fatty acid content; high unsaturated fatty acid and low polyunsaturated fatty acid content; or high unsaturated fatty acid, high monounsaturated fatty acid, and low polyunsaturated fatty acid content, relative to a product produced from a control plant or plant part. In some embodiments, the plant product (e.g., oil, protein composition) comprises high monounsaturated fatty acid to polyunsaturated fatty acid composition relative to a control product.

A control plant, plant part, or plant product can be a plant, plant part, or product produced therefrom having normal (e.g., not reduced) FAD, LOX, and/or KAS activity; not having changes that would alter (e.g., reduce) FAD, LOX, and/or KAS activity; not having mutation in FAD, LOX, and/or KAS genes provided herein; or commodity plant, plant part, or product therefrom. As an example and without limitation, a control (e.g., reference, commodity) plant, plant part, or plant products (e.g., oil), e.g., without reduced FAD, LOX, and/or KAS activity, may have fatty acid compositions comprising palmitic acid, stearic acid, oleic acid, linoleic acid, and linolenic acid of about 10-15%, 4-6%, 15-30%, 45-55%, and 10-15%, respectively. For example, a control pea plant or pea plant part, or plant products (e.g., oil) e.g., without reduced FAD, LOX, and/or KAS activity, may have fatty acid compositions comprising palmitic acid, stearic acid, oleic acid, linoleic acid, and linolenic acid of about 13%, 4%, 29%, 46%, and 9%, respectively. A control soybean plant or plant part, or plant products (e.g., oil), e.g., without reduced FAD, LOX, and/or KAS activity, may have fatty acid compositions comprising palmitic acid, stearic acid, oleic acid, linoleic acid, and linolenic acid of about 10%, 4%, 18%, 55%, and 13%, respectively (Clemente & Cahoon 2009, Plant Physiol. 151:1030-1040).

A plant, plant part, or plant product (e.g., oil) that has “low linolenic acid” content as used herein refers to a plant, plant part, or plant product (e.g., oil) having a less linolenic acid content as compared to a reference sample (e.g., control, without FAD and/or LOX gene mutation, commodity sample) of plant, plant part, or plant product. A plant, plant part, or plant product (e.g., oil) that has “low linolenic acid” content includes a plant, plant part, or plant product (e.g., oil) that has lower linolenic acid content, expressed as percent of total fatty acids, as compared to a control without the mutation, with the difference (by subtraction) of at least about 1%, 2%, 3%, 4%, 5%, 6%, 7%, 8%, 9%, 10%, 15%, 20%, 25%, or 30%. A plant, plant part, or plant product (e.g., oil) that has “low linolenic acid” content also includes a plant, plant part, or plant product (e.g., oil) that has a linolenic acid content of about 1% to about 10%, e.g., about 4-10%, 1-2%, 2-3%, 3-4%, 4-5%, 5-6%, 6-7%, 7-8%, 8-9%, or 9-10%; or 15% or less; 14% or less; 13% or less; 12% or less, 11% or less; 10% or less; 9% or less; 8% or less; 7% or less; 6% or less; 5% or less; 4% or less; 3% or less; 2% or less; or 1% or less (of total fatty acids) by weight.

A plant, plant part, or plant product (e.g., oil) that has “low linoleic acid” content as used herein refers to a plant, plant part, or plant product (e.g., oil) having a less linoleic acid content as compared to a reference sample (e.g., control, without FAD and/or LOX gene mutation, commodity sample) of plant, plant part, or plant product. A plant, plant part, or plant product (e.g., oil) that has “low linoleic acid” content includes a plant, plant part, or plant product (e.g., oil) that has lower linoleic acid content, expressed as percent of total fatty acids, as compared to a control without the mutation, with the difference (by subtraction) of at least about 1%, 2%, 3%, 4%, 5%, 6%, 7%, 8%, 9%, 10%, 15%, 20%, 25%, 30%, 35%, 40%, 45%, or 50%. A plant, plant part, or plant product (e.g., oil) that has “low linoleic acid” content also includes a plant, plant part, or plant product (e.g., oil) that has a linoleic acid content of about 20% to about 45%, e.g., about 35-45%, 20-30%, 30-35%, 35-40%, or 40-45%; 50% or less; 45% or less; 40% or less; 35% or less, 30% or less; 25% or less; 20% or less; 15% or less; 10% or less; or 5% or less (of total fatty acids) by weight.

A plant, plant part, or plant product (e.g., oil) that has “low polyunsaturated acid” or “low linolenic acid and linoleic acid” content as used herein refers to a plant, plant part, or plant product (e.g., oil) having a less polyunsaturated acid content, or a less linolenic acid plus linoleic acid content, as compared to a reference sample (e.g., control, without FAD and/or LOX gene mutation, commodity sample) of plant, plant part, or plant product. A plant, plant part, or plant product (e.g., oil) that has “low polyunsaturated acid” or “low linolenic acid and linoleic acid” content includes a plant, plant part, or plant product (e.g., oil) that has lower polyunsaturated acid content, or lower linolenic acid plus linoleic acid content, expressed as percent of total fatty acids, as compared to a control without the mutation, with the difference (by subtraction) of at least about 1%, 2%, 3%, 4%, 5%, 6%, 7%, 8%, 9%, 10%, 15%, 20%, 25%, 30%, 35%, 40%, 45%, or 50%. A plant, plant part, or plant product (e.g., oil) that has “low polyunsaturated acid” or “low linolenic acid and linoleic acid” content also includes a plant, plant part, or plant product (e.g., oil) that has a polyunsaturated fatty acid content or a linolenic acid plus linoleic acid content of about 30% to about 55%, e.g., about 45-55%, 30-35%, 35-40%, 40-45%, 45-55%, 50-55%, 60% or less, 55% or less, 50% or less, 45% or less, 40% or less, 35% or less, or 30% or less (of total fatty acids) by weight.

A plant, plant part, or plant product (e.g., oil) that has “high oleic acid” or “high monounsaturated fatty acid” content as used herein refers to a plant, plant part, or plant product (e.g., oil) having a greater monounsaturated fatty acid content (e.g., a greater oleic acid content) as compared to a reference sample (e.g., control, without FAD and/or LOX gene mutation, commodity sample) of plant, plant part, or plant product. A plant, plant part, or plant product (e.g., oil) that has “high oleic acid” or “high monounsaturated fatty acid” content includes a plant, plant part, or plant product (e.g., oil) that has higher monounsaturated fatty acid (e.g., oleic acid) content, expressed as percent of total fatty acids, as compared to a control without the mutation, with the difference (by subtraction) of at least about at least about 1%, 2%, 3%, 4%, 5%, 6%, 7%, 8%, 9%, 10%, 15%, 20%, 25%, 30%, 35%, 40%, 45%, or 50%. A plant, plant part, or plant product (e.g., oil) that has “high oleic acid” or “high monounsaturated fatty acid” content also includes a plant, plant part, or plant product (e.g., oil) that has a monounsaturated acid content of about 30% to about 55%, e.g., about 30-40%, 30-35%, 35-40%, 40-45%, 45-55%, 50-55%, 60% or less, 55% or less, 50% or less, 45% or less, 40% or less, 35% or less, or 30% or less (of total fatty acids) by weight.

A plant, plant part, or plant product (e.g., oil) that has “high oleic acid”, low linoleic acid, and/or low linolenic acid content, or “high monounsaturated to polyunsaturated fatty acid composition”, also referred to as a “HOLL” fatty acid composition, “HOLL” product (e.g., oil), or “HOLL” phenotype, as used herein refers to a plant, plant part, or plant product (e.g., oil) having a greater monounsaturated fatty acid content (e.g., a greater oleic acid content), a less polyunsaturated fatty acid content (e.g., a less linoleic acid and/or linolenic acid content), and/or a greater monounsaturated to polyunsaturated fatty acid composition (e.g., a greater oleic acid to linoleic and/or linolenic acid composition), as compared to a reference sample (e.g., control, commodity sample) of plant, plant part, or plant product. An “HOLL” plant, plant part, or plant product (e.g., oil) includes a plant, plant part, or plant product (e.g., oil) that has one or more characteristics of “high oleic acid”, “high monounsaturated fatty acid”, “low linoleic acid”, “low linolenic acid”, “low linolenic plus linoleic acid”, “low polyunsaturated fatty acid” content, and “high monounsaturated to polyunsaturated fatty acid composition” provided herein. An “HOLL” plant, plant part, or plant product (e.g., oil) also includes a plant, plant part, or plant product (e.g., oil) that has a linolenic acid content of about 1% to about 10% (e.g., about 1-4%, 4-5%, 5-6%, 6-7%, 7-8%, 8-9%, 9-10%), an oleic acid content of about 30% to about 55% (e.g., about 30%-35%, 35%-40%, 40-45%, 45-50%, 50-55%), and a linoleic plus linolenic acid content of about 30% to 55% (e.g., about 30-40%, 40-45%, 45%-50%, 50%-55%) (of total fatty acids, by weight).

A plant, plant part, or plant product (e.g., oil) that has a “high palmitic acid” or “high saturated fatty acid” phenotype or content as used herein refers to a plant, plant part, or plant product (e.g., oil) having a greater saturated fatty acid content (e.g., greater palmitic acid content) as compared to a reference sample (e.g., control, without FAD, LOX, and/or KAS mutation, commodity sample) of plant, plant part, or plant product. A plant, plant part, or plant product (e.g., oil) with a “high palmitic acid” o “high saturated fat” phenotype or content includes a plant, plant part, or plant product (e.g., oil) that has higher saturated fatty acid (e.g., higher palmitic acid) content, expressed as percent of total fatty acids, as compared to a control plant, plant part, or plant product (e.g., without mutation), with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A plant, plant part, or plant product (e.g., oil) that has “high palmitic acid” or “high saturated fatty acid” content also includes a plant, plant part, or plant product (e.g., oil) that has a palmitic acid or saturated fatty acid content of about 10% to about 50%, e.g., about 15-30%, 15-17.5%, 17.5-20%, 20-22.5%, 22.5-25%, 25-27.5%, 27.5-30%, 30-40%, 40-50%, 10% or more, 15% or more, 20% or more, 25% or more, 30% or more, 35% or more, 40% or more, 45% or more, or 50% or more (of total fatty acids) by weight.

A plant, plant part, or plant product (e.g., oil) with a “high saturated fatty acid” (e.g., palmitic acid, stearic acid), “high monounsaturated fatty acid” (e.g., oleic acid), and/or “low polyunsaturated fatty acid” (e.g., linoleic and linolenic acid) phenotype, also referred to as a “HPHOLL” phenotype, as used herein refers to a plant, plant part, or plant product having a greater saturated fatty acid content (e.g., a greater palmitic acid or stearic acid content), a greater monounsaturated fatty acid content (e.g., a greater oleic acid content), a less polyunsaturated fatty acid content (e.g., a less linoleic acid and/or linolenic acid content), a greater saturated to unsaturated fatty acid composition, or a greater saturated plus monounsaturated to polyunsaturated fatty acid composition, as compared to a reference sample of soybean plant or seed. An “HPHOLL” soybean plant, oil, or seed includes a plant, plant part, or plant product (e.g., oil) that has one or more characteristics of “high palmitic acid”, “high stearic acid”, “high palmitic plus stearic acid”, “high saturated fatty acid”, “high oleic acid”, “high monounsaturated fatty acid”, “low linoleic acid”, “low linolenic acid”, “low linolenic plus linoleic acid”, “low polyunsaturated fatty acid”, “high monounsaturated to polyunsaturated fatty acid”, and “high saturated plus monounsaturated to polyunsaturated fatty acid” content or composition provided herein. An HPHOLL plant, plant part, or plant product also includes a plant, plant part, or plant product that has a saturated fatty acid content of about 17.5% to about 35% (of total fatty acids) and a polyunsaturated fatty acid content of about 5% to 30% (of total fatty acids). An HPHOLL plant, plant part, or plant product also includes a plant, plant part, or plant product that has a palmitic acid content of at least 15%, 20%, 25%, or 30% (of total fatty acids) by weight; a stearic acid content of at least 2.5%, 3.0%, or 3.5% (of total fatty acids) by weight; an oleic acid content of at least 35%, 40%, 45%, 50%, 55%, 60%, 65%, 70%, 75%, or 80% (of total fatty acids) by weight; a linoleic acid content of 5% or less, 10% or less, 15% or less, 20% or less, 25% or less (of total fatty acids) by weight; a linolenic acid content of 1% or less, 2% or less, 3% or less, 4% or less, 5% or less (of total fatty acids) by weight; a saturated fatty acid content of at least 15%, 20%, 25%, 30%, or 35% (of total fatty acids) by weight; a saturated plus monounsaturated fatty acid content of at least 50%, 55%, 60%, 70%, 75%, 80%, 85%, 90%, 95%, 96%, 97%, 98%, or 99% (of total fatty acids) by weight. In particular embodiment an HPHOLL plant, plant part, or plant product comprises a palmitic acid content of about 15% to about 30%, a stearic acid content of about 2.5% to about 3.5%, an oleic acid content of about 35% to about 80%, a linoleic acid content of about 5% to 25%, and/or a linolenic acid content of about 1% to about 5% by weight, as normalized to total fatty acids (which represents 100%).

Amount or levels of total fatty acids and specific fatty acids can be measured by any methods for measuring fatty acid amount or levels, including gas chromatography-mass spectrometry (GC-MS) optionally with certain modifications (e.g., with or without initial lipid extraction, with or without isotope labeling of analytes). Fatty acid composition (e.g., percentage of specific fatty acids normalized to total fatty acids) can be calculated based on the amount or concentration of total fatty acids and specific fatty acids in the sample.

In some embodiments, level of linolenic acid in plant products (e.g., protein composition, oil), e.g., obtained from a plant with a decreased LOX or FAD activity, a plant with a mutated LOX (e.g., LOX-2, LOX-3) and/or FAD2B, FAD3C, and/or FAD3D gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, as compared to a control product obtained from a control plant (e.g., without decreased FAD or LOX activity, without mutation). In some embodiments, the plant products (e.g., oil, protein compositions) produced from plants or plant parts provided herein can have lower linolenic acid content, expressed as percent dry weight of total fatty acids, as compared to a control product (e.g., oil, protein composition) produced from plants or plant parts without the mutation, and the difference (by subtraction) can be at least about 1%, 2%, 3%, 4%, 5%, 6%, 7%, 8%, 9%, 10%, 15%, 20%, 25%, 30%, 35%, 40%, 45%, or 50%. In some embodiments, the linolenic acid content in plant products (e.g., plant oil, protein compositions) provided herein is at least about 1%, 2%, 3%, or 4% less compared to that in a control plant product (e.g., without decreased FAD or LOX activity), expressed as difference in % of total fatty acids in dry weight. In some embodiments, the plant products (e.g., oil, protein compositions) provided herein can comprise a linolenic acid content of about 1% to about 10%, e.g., about 4-10%, 1-2%, 2-3%, 3-4%, 4-5%, 5-6%, 6-7%, 7-8%, 8-9%, or 9-10%; or 15% or less; 14% or less; 13% or less; 12% or less, 11% or less; 10% or less; 9% or less; 8% or less; 7% or less; 6% or less; 5% or less; 4% or less; 3% or less; 2% or less; or 1% or less (of total fatty acids) by weight. Suitable percentage ranges for linolenic acid content in plant products of the present invention also include ranges in which the lower limit is selected from the following percentages: 0.1, 0.5, 1, 2, 3, 4, 5, 6, 7, 8, 9, or 10 percent; and the upper limit is selected from the following percentages: 1, 2, 3, 4, 5, 6, 7, 8, 9, 10, 11, 12, 13, 14, 15, 16, 17, 18, 19, or 20 percent.

In some embodiments, level of polyunsaturated fatty acids (e.g., combined linolenic acid and linoleic acid) in plant products (e.g., protein composition, oil), e.g., obtained from a plant with a mutated LOX (e.g., LOX-2, LOX-3) and/or FAD2B, FAD3C, and/or FAD3D gene can be reduced as compared to a control product obtained from a control plant (e.g., without mutation). In some embodiments, the plant products (e.g., oil, protein compositions) produced from plants or plant parts with mutation disclosed herein can have lower polyunsaturated fatty acid content, expressed as percent of total fatty acids, as compared to a control product (e.g., oil, protein composition) produced from plants or plant parts without the mutation, and the difference (by subtraction) can be at least about 1%, 2%, 3%, 4%, 5%, 6%, 7%, 8%, 9%, 10%, 15%, 20%, 25%, 30%, 35%, 40%, 45%, or 50%. In some embodiments, the polyunsaturated acid content in plant products (e.g., plant oil, protein compositions) provided herein is at least about 1%, 2%, 3%, 4%, 5%, 6%, 7%, 8%, 9%, 10%, 11%, 12%, 13%, 14%, or 15% less, or about 1-5% less; 5-10% less, 10-15% less, or more than 15% less, relative to the polyunsaturated acid content in a control product produced from a control plant or plant part (e.g., without mutation), expressed as difference in % of total fatty acids in dry weight. In some embodiments, the plant products (e.g., oil, protein compositions) provided herein can comprise a polyunsaturated acid content of about 30% to about 55%, e.g., about 30-35%, 35-40%, 40-45%, 45-55%, 50-55%, 60% or less, 55% or less, 50% or less, 45% or less, 40% or less, 35% or less, or 30% or less (of total fatty acids) by weight. Suitable percentage ranges for polyunsaturated acid content in plant products of the present invention also include ranges in which the lower limit is selected from the following percentages: 20, 25, 30, 35, 40, 45, or 50 percent; and the upper limit is selected from the following percentages: 30, 35, 40, 45, 50, 55, or 60 percent.

In some embodiments, level of oleic acids in plant products (e.g., protein composition, oil), e.g., obtained from a plant with a mutated LOX (e.g., LOX-2, LOX-3) and/or FAD2B, FAD3C, and/or FAD3D gene can be increased as compared to a control product obtained from a control plant (e.g., without mutation). In some embodiments, the plant products (e.g., oil, protein compositions) produced from plants or plant parts with mutation disclosed herein can have higher oleic acid content, expressed as percent of total fatty acids, as compared to a control product (e.g., oil, protein composition) produced from plants or plant parts without the mutation, and the difference (by subtraction) can be at least about 1%, 2%, 3%, 4%, 5%, 6%, 7%, 8%, 9%, 10%, 15%, 20%, 25%, 30%, 35%, 40%, 45%, or 50%. In some embodiments, the oleic acid content in plant products (e.g., plant oil, protein compositions) provided herein is at least about 1%, 2%, 3%, 4%, 5%, 6%, 7%, 8%, 9%, 10%, 11%, 12%, 13%, 14%, or 15% more (by subtraction), or about 1-5% more; 5-10% more, 10-15% more, or more than 15% more (by subtraction) compared to a control product (e.g., oil, protein composition) produced from a control plant or plant part (e.g., without mutation), expressed as difference in % dry weight of total fatty acids. In some embodiments, the plant products (e.g., oil) provided herein have at least 4% increase in oleic acid content compared to a control product, expressed as difference in % dry weight of total fatty acids. In some embodiments, the plant products (e.g., oil, protein compositions) provided herein can comprise an oleic acid content of about 30% to about 60%, e.g., about 30-35%, 35-40%, 40-45%, 45-55%, 50-55%, 55-60%, 60% or more, 55% or more, 50% or more, 45% or more, 40% or more, 35% or more, or 30% or more (of total fatty acids) by weight. Suitable percentage ranges for oleic acid content in oils of the present invention also include ranges in which the lower limit is selected from the following percentages: 25, 30, 31, 32, 33, 34, 35, 36, 37, 38, 39, 40, 41, 42, 43, 44, 45, 46, 47, 48, 49, 50, 51, 52, 53, 54, or 55 percent; and the upper limit is selected from the following percentages: 30, 31, 32, 33, 34, 35, 36, 37, 38, 39, 40, 41, 42, 43, 44, 45, 46, 47, 48, 49, 50, 51, 52, 53, 54, 55, 60, 65, or 70 percent.

In some embodiments, plant products (e.g., oil, protein composition) provided herein can comprise linolenic acid content of 1-10% and oleic acid content of 30-60%, for example, 1-4% linolenic acid and 50-60% oleic acid; 1-4% linolenic acid and 40-50% oleic acid; 1-4% linolenic acid and 35-40% oleic acid; 1-4% linolenic acid and 30-35% oleic acid; 4-7% linolenic acid and 50-60% oleic acid; 4-7% linolenic acid and 40-50% oleic acid; 4-7% linolenic acid and 35-40% oleic acid; 4-7% linolenic acid and 30-35% oleic acid; 4-7% linolenic acid and 30-35% oleic acid; 7-10% linolenic acid and 50-60% oleic acid; 7-10% linolenic acid and 40-50% oleic acid; 7-10% linolenic acid and 35-40% oleic acid; or 7-10% linolenic acid and 30-35% oleic acid. In some embodiments, plant products (e.g., oil, protein composition) provided herein can comprise polyunsaturated acid content of 30-55% and oleic acid content of 30-60%, for example, 30-40% polyunsaturated acid and 50-60% oleic acid; 30-40% polyunsaturated acid and 40-50% oleic acid; 30-40% polyunsaturated acid and 35-40% oleic acid; 30-40% polyunsaturated acid and 30-35% oleic acid; 40-45% polyunsaturated acid and 50-60% oleic acid; 40-45% polyunsaturated acid and 40-50% oleic acid; 40-45% polyunsaturated acid and 35-40% oleic acid; 40-45% polyunsaturated acid and 30-35% oleic acid; 45-50% polyunsaturated acid and 50-60% oleic acid; 45-50% polyunsaturated acid and 40-50% oleic acid; 45-50% polyunsaturated acid and 35-40% oleic acid; 45-50% polyunsaturated acid and 30-35% oleic acid; 50-55% polyunsaturated acid and 50-60% oleic acid; 50-55% polyunsaturated acid and 40-50% oleic acid; 50-55% polyunsaturated acid and 35-40% oleic acid; or 50-55% polyunsaturated acid and 30-35% oleic acid.

In specific embodiments, the plant product (e.g., oil, protein composition) provided herein comprises at least about 4% increase in oleic acid content and/or at least about 4% decrease in linoleic plus linolenic acid content relative to oil produced from a control plant or plant part, expressed as difference in % dry weight of total fatty acids. In some embodiments, the plant product (e.g., oil, protein composition) provided herein comprises a linolenic acid content of about 4% to about 10% (e.g., about 4-5%, 5-6%, 6-7%, 7-8%, 8-9%, 9-10%), an oleic acid content of about 30% to about 40% (e.g., about 30%-35%, 35%-40%), and a linoleic plus linolenic acid content of about 45% to 55% (e.g., about 45%-50%, 50%-55%) (of total fatty acids, by weight).

Also provided herein is plant oil having a high oleic acid, low linoleic acid, and/or low linolenic acid content; or a high monounsaturated fatty acid to polyunsaturated fatty acid content, which is also referred to as “HOLL” oil herein. Such HOLL oil includes plant oil produced from plants or plant parts with a decreased LOX and/or FAD activity provided herein, and may contain a mutated LOX and/or FAD gene (e.g., LOX-2, LOX-3, FAD2, FAD3) or fragment thereof. In some embodiments, the HOLL oil provided herein does not contain such mutation. The HOLL oil of the present disclosure can be produced by any methods. For example, the HOLL oil of the present disclosure can be produced, exclusively or nonexclusively: from plants or plant parts (e.g., seeds) that have decreased LOX and/or FAD activity; from plants or plant parts (e.g., seeds) that contain a mutation of a LOX and/or FAD gene disclosed herein; from plants or plant parts (e.g., seeds) that do not contain a mutation disclosed herein; from plants or plant parts (e.g., seeds) produced according to the methods provided herein, or from plants or plant parts (e.g., seeds) not produced or selected according to the methods provided herein. The HOLL plant oil provided herein can have any or all characteristics of fatty acid compositions provided herein (e.g., high oleic acid content; low linoleic acid content; low linolenic acid content; high oleic acid and low linoleic acid content; high oleic acid and low linolenic acid content; low linoleic acid and low linolenic acid content; high oleic acid, low linoleic acid, and low linolenic acid content; high monounsaturated to polyunsaturated fatty acid content).

Also provided herein is plant oil having a high palmitic acid, high oleic acid, low linoleic acid, and/or low linolenic acid content; or a high saturated fatty acid, high monounsaturated fatty acid, and low polyunsaturated fatty acid content, which is also referred to as “HPHOLL” oil herein. Such HPHOLL oil includes plant oil produced from plants or plant parts with a decreased LOX, FAD, and/or KAS activity provided herein, and may contain a mutated LOX, FAD, and/or KAS gene (e.g., LOX-2, LOX-3, FAD2, FAD3, KAS II) or fragment thereof. In some embodiments, the HPHOLL oil provided herein does not contain such mutation. The HPHOLL oil of the present disclosure can be produced by any methods. For example, the HPHOLL oil of the present disclosure can be produced, exclusively or nonexclusively: from plants or plant parts (e.g., seeds) that have decreased LOX, FAD, and/or KAS activity; from plants or plant parts (e.g., seeds) that contain a mutation of a LOX, FAD, and/or KAS gene disclosed herein; from plants or plant parts (e.g., seeds) that do not contain a mutation disclosed herein; from plants or plant parts (e.g., seeds) produced according to the methods provided herein, or from plants or plant parts (e.g., seeds) not produced or selected according to the methods provided herein. The HPHOLL plant oil provided herein can have any or all characteristics of fatty acid compositions provided herein (e.g., high palmitic acid content; high oleic acid content; low linoleic acid content; low linolenic acid content; high oleic acid and low linoleic acid content; high oleic acid and low linolenic acid content; low linoleic acid and low linolenic acid content; high oleic acid, low linoleic acid, and low linolenic acid content; high palmitic acid and high oleic acid content; high palmitic acid and low linoleic acid content; high palmitic acid and low linolenic acid content; high palmitic acid, high oleic acid, and low linoleic acid content; high palmitic acid, high oleic acid, and low linolenic acid content; high palmitic acid, low linoleic acid, and low linolenic acid content; high palmitic acid, high oleic acid, low linoleic acid, and low linolenic acid content, high unsaturated fatty acid content; high unsaturated fatty acid and high monounsaturated fatty acid content; high unsaturated fatty acid and low polyunsaturated fatty acid content; or high unsaturated fatty acid, high monounsaturated fatty acid, and low polyunsaturated fatty acid content; or high monounsaturated to polyunsaturated fatty acid content).

Also provided herein are food and/or beverage products containing a protein composition or oil described herein, such as a protein composition obtained from a plant with a mutated FAD gene. Such food and/or beverage products include, without limitation, protein shakes, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc.). A food and/or beverage product that contains a protein composition obtained from a plant with a mutated FAD gene can have improved flavor characteristics (aspects described as, e.g., overall aroma, overall flavor impact, beany yellow pea, pyrazine, cereal grain, green grassy green pea, nutty, cardboard, malty, salt, bitter, umami, astringent, or chalky), compared to a similar or comparable food and/or beverage product that contains a protein composition obtained from a control plant.

### a. Plant Products comprising Nucleic Acid Molecule having a Mutated Sequence of LOX-2, LOX-3, FAD2, or FADS Gene or Fragment

In some embodiments, plant products (e.g., oil, protein compositions) provided herein are produced from plants or plant parts having one or more mutated LOX or FAD genes (e.g., LOX-2, LOX-3, FAD2B, FAD3A, FAD3B). In some such embodiments, the plant products provided herein can comprise one or more nucleic acid molecules comprising a nucleic acid sequence of a mutated LOX or FAD gene (e.g., LOX-2, LOX-3, FAD2B, FAD3A, FAD3B) or fragment thereof. A “fragment”, as used herein, refers to a nucleic acid molecule comprising a contiguous nucleic acid sequence of any part of a gene. A fragment of a mutated gene can comprise the full or partial sequence of the region of the gene where the mutation is located. The presence of a mutated gene or fragment thereof in a plant product can be detected by any standard methods for detecting mutations in nucleic acid molecules in a plant sample, including PCR and quantitative real-time PCR. For instance, the presence of a mutated gene or fragment thereof in plant oil can be detected by methods for detecting DNA fragments in samples, including PCR and quantitative real-time PCR, as described for example in Duan at al. 2021 Food Sci Biotechnol 30(1):129-135, the entirety of which is herein incorporated by reference.

In some embodiments, plant products (e.g., oil, protein composition) provided herein comprises one or more nucleic acid molecules each comprising a nucleic acid sequence of a mutated LOX-2, LOX-3, FAD2, or FAD3 gene or fragment thereof. Such mutation may cause reduced expression or activity of the gene comprising the mutation. In some embodiments, said one or more nucleic acid molecules in the oil or protein composition each comprise a nucleic acid sequence of: (i) a mutated LOX-2 gene or a fragment thereof comprising a deletion of nucleotides 1521 through 1531 of SEQ ID NO: 10; (ii) a mutated LOX-2 gene or a fragment thereof comprising a deletion of nucleotides 1523 through 1530 of SEQ ID NO: 10; (iii) a mutated LOX-3 gene or a fragment thereof comprising a deletion of nucleotides 1129 through 1156 of SEQ ID NO: 27; (iv) a mutated FAD2B gene or a fragment thereof comprising a deletion of nucleotides 59 through 66 of SEQ ID NO: 36; (v) a mutated FAD2B gene or a fragment thereof comprising a deletion of nucleotides 60 through 61 of SEQ ID NO: 36; (vi) a mutated FAD3C gene or a fragment thereof comprising a deletion of nucleotides 457 through 464 of SEQ ID NO: 46; (vii) a mutated FAD3C gene or a fragment thereof comprising a deletion of nucleotides 416 through 464 of SEQ ID NO: 46; (viii) a mutated FAD3D gene or a fragment thereof comprising a deletion of nucleotides 775 through 779 of SEQ ID NO: 56; or (ix) a mutated FAD3D gene or a fragment thereof comprising a deletion of nucleotides 745 through 851 of SEQ ID NO: 56.

### 2.5 Reducing FAD Activity to Improve Flavor Characteristics or Modify Fatty Acid Profile

Provided herein are methods for reducing the function and/or expression of a FAD (e.g., FAD2B, FAD3C, FAD3D) protein in a plant or plant part. In particular, methods of the present disclosure can reduce function and/or expression of FAD protein in a plant or plant part by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, methods of the present disclosure can reduce expression and/or function of FAD protein in a plant or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In specific embodiments, the FAD protein is a FAD2B protein. In some embodiments, the FAD protein is a FAD3C protein. In other embodiments, the FAD protein is a FAD3D protein.

Also provided herein are methods of decreasing the level of linolenic acid in a plant (e.g., Pisum sativum plant) or plant part when compared to a control plant or plant part. In particular, methods comprise decreasing the activity of one or more genes in the plant with a mutant gene selected from the group consisting of LOX-2, LOX-3, FAD2, and FAD3. In some embodiments, methods provided herein comprise decreasing the activity of LOX-2 and FAD3C genes in the plant with a mutant LOX-2 and FAD3C genes. In some embodiments, methods provided herein comprise decreasing the activity of LOX-2 and FAD2B genes in the plant with a mutant LOX-2 and FAD2B genes. In some embodiments, methods provided herein comprise decreasing the activity of LOX-2 and FAD3D genes in the plant with a mutant LOX-2 and FAD3C genes. The decreased activity in the plant or plant part is by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part expressing one or more WT LOX-2, WT LOX-3, WT FAD2, and/or WT FAD3 genes. Additionally, or alternatively, methods of the present disclosure can decrease the level of linolenic acid in a plant (e.g., Pisum sativum plant) or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part.

Also provided herein are methods of increasing the level of oleic acid in a plant (e.g., Pisum sativum plant) or plant part when compared to a control plant or plant part. In particular, methods comprise decreasing the activity of one or more genes in the plant with a mutant gene selected from the group consisting of LOX-2, LOX-3, FAD2, and FAD3. In some embodiments, methods provided herein comprise decreasing the activity of LOX-2 and FAD3C genes in the plant with a mutant LOX-2 and FAD3C genes. In some embodiments, methods provided herein comprise decreasing the activity of LOX-2 and FAD2B genes in the plant with a mutant LOX-2 and FAD2B genes. In some embodiments, methods provided herein comprise decreasing the activity of LOX-2 and FAD3D genes in the plant with a mutant LOX-2 and FAD3C genes. The decreased activity in the plant or plant part is by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part expressing one or more WT LOX-2, WT LOX-3, WT FAD2, and/or WT FAD3 genes. Additionally, or alternatively, methods of the present disclosure can increase the level of oleic acid in a plant (e.g., Pisum sativum plant) or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant (e.g., Pisum sativum plant) or plant part.

Also provided herein are methods of increasing the level of palmitic acid and/or saturated fatty acid in a plant (e.g., Pisum sativum plant) or plant part when compared to a control plant or plant part. In particular, methods comprise decreasing the activity of one or more KAS genes (e.g., KAS II) in the plant, e.g., by introducing mutation to the gene. In some embodiments, methods provided herein comprise decreasing the activity of LOX and/or FAD genes and a KAS gene in the plant by introducing mutation to the LOX-2 and/or FAD genes and the KAS gene. In some embodiments, methods provided herein comprise decreasing the activity of KAS II gene and one or more of FAD2B, FAD3C, and FAD3D genes; KAS II gene and one or more of LOX-2 and LOX-3 genes; KAS II gene, one or more of FAD2B, FAD3C, and FAD3D genes, and one or more of LOX-2 and LOX-3 genes, by introducing a mutation to the respective genes. The decreased activity in the plant or plant part of the respective genes can be by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part expressing a control (e.g., wild-type) genes. Methods of the present disclosure can increase the level of palmitic acid and/or saturated fatty acid in a plant (e.g., Pisum sativum plant or Glycine max plant) or plant part by at least 10%, 15%, 20%, 25%, 30%, 35%, 40%, 45%, 50%, 55%, 60%, 65%, 70%, 75%, 80%, 85%, 90%, 95%, 100%, 150%, 200%, or more, as compared to a control plant (e.g., Pisum sativum plant or Glycine max plant) or plant part.

### 2.6 Mutation of LOX, FAD Genes

Activity of a LOX (e.g., LOX-2, LOX-3) and/or FAD (e.g., FAD2B, FAD3C, FAD3D) protein in a plant or plant part can be reduced by reducing the expression of LOX and/or FAD proteins. Methods of the present disclosure can reduce expression of LOX (e.g., full-length LOX) protein in a plant or plant part by mutating a LOX gene, i.e., a gene encoding LOX protein. Methods of the present disclosure can also reduce expression of FAD (e.g., full-length FAD) protein in a Pisum sativum plant or plant part by mutating a LOX gene, i.e., a gene encoding LOX protein.

Described herein are methods for mutating a LOX and/or FAD gene in a plant cell or plant part, e.g., by one or more (e.g., 1, 2, 3, 4, 5, 6, 7, 8, 9, 10, or more) insertions, substitutions, or deletions. For example, methods of the present disclosure can result in mutation of LOX-2 gene (i.e., a gene encoding a LOX-2 protein) in the genome of cells or parts of a plant by one or more nucleic acid insertions, substitutions, or deletions in the LOX-2 gene. In some instances, methods of the present disclosure can result in mutation of LOX-3 gene (i.e., a gene encoding a LOX-3 protein) in the genome of cells or parts of a plant by one or more nucleic acid insertions, substitutions, or deletions in the LOX-3 gene. In some embodiments, methods of the present disclosure can result in mutation of FAD2B gene (i.e., a gene encoding a FAD2B protein) in the genome of cells or parts of a plant by one or more nucleic acid insertions, substitutions, or deletions in the FAD2B gene. In some embodiments, methods of the present disclosure can result in mutation of FAD3C gene (i.e., a gene encoding a FAD3C protein) in the genome of cells or parts of a plant by one or more nucleic acid insertions, substitutions, or deletions in the FAD3C gene. In some embodiments, methods of the present disclosure can result in mutation of FAD3D gene (i.e., a gene encoding a FAD3D protein) in the genome of cells or parts of a plant by one or more nucleic acid insertions, substitutions, or deletions in the FAD3D gene.

In some embodiments, methods of the present disclosure can result in mutation of LOX-2 gene (i.e., a gene encoding a LOX-2 protein) and LOX-3 gene (i.e., a gene encoding a LOX-3 protein) in the genome of cells or parts of a plant by one or more nucleic acid insertions, substitutions, or deletions in the LOX-2 and LOX-3 genes. In some embodiments, methods of the present disclosure can result in mutation of LOX-2 gene (i.e., a gene encoding a LOX-2 protein) and FAD3C gene (i.e., a gene encoding a FAD3C protein) in the genome of cells or parts of a plant by one or more nucleic acid insertions, substitutions, or deletions in the LOX-2 and FAD3C genes.

Mutation can be any change in the nucleic acid sequence of a gene, such as LOX and/or FAD gene. Non-limiting examples of mutation of LOX and/or FAD gene comprise insertions, deletions, duplications, substitutions, inversions, and translocations of any nucleic acid sequence of the LOX and/or FAD gene, regardless of how the mutation is brought about and regardless of how or whether the mutation alters the functions or interactions of the nucleic acid. For example, a mutation may produce, without limitation, altered enzymatic activity of a ribozyme, altered base pairing between nucleic acids (e.g., RNA interference interactions, DNA-RNA binding, etc.), altered mRNA folding stability, and/or how a nucleic acid interacts with polypeptides (e.g., DNA-transcription factor interactions, RNA-ribosome interactions, gRNA-endonuclease reactions, etc.). A mutation in LOX and/or FAD gene might result in the production of LOX and/or FAD protein with altered amino acid sequences (e.g., missense mutations, nonsense mutations, frameshift mutations, etc.) and/or the production of LOX and/or FAD protein with the same amino acid sequence (e.g., silent mutations). Certain synonymous mutations in LOX gene may create no observed change in the plant, while others that encode for an identical protein sequence can nevertheless result in an altered plant phenotype (e.g., due to codon usage bias, altered secondary protein structures, etc.). Mutations in a LOX and/or FAD gene may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements, such as tRNAs, ribozymes, ribosome components, and microRNAs.

Methods disclosed herein are not limited to certain techniques of mutagenesis of LOX and/or FAD gene. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g., CRISPR-like technologies, TALENs, zinc finger nucleases, and meganucleases), ionizing radiation (e.g., ultraviolet and/or gamma rays), temperature alterations, long-term seed storage, tissue culture conditions, targeting induced local lesions in a genome, sequence-targeted and/or random recombinases, etc. It is anticipated that new methods of creating a mutation in LOX and/or FAD gene of a plant will be developed and yet fall within the scope of the claimed invention when used with the teachings described herein.

Similarly, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g., using a gene-gun), Agrobacterium infection and/or infection by other bacterial species capable of transferring DNA into plants (e.g., Ochrobactrum sp., Ensifer sp., Rhizobium sp.), viral infection, and other techniques can be used to deliver nucleic acid sequences into a plant described herein. Methods disclosed herein are not limited to any size of nucleic acid sequences that are introduced, and thus one could introduce a nucleic acid comprising a single nucleotide (e.g., an insertion) into a nucleic acid of the plant and still be within the teachings described herein. Nucleic acids introduced in substantially any useful form, for example, on supernumerary chromosomes (e.g., B chromosomes), plasmids, vector constructs, additional genomic chromosomes (e.g., substitution lines), and other forms is also anticipated. It is envisioned that new methods of introducing nucleic acids into plants and new forms or structures of nucleic acids will be discovered and yet fall within the scope of the claimed invention when used with the teachings described herein.

Methods disclosed herein include conferring desired traits to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain non-limiting embodiments, the trait of decreased LOX-2 and/or LOX-3 function is conferred to the plant by introducing a nucleotide sequence (e.g., using plant transformation methods) that encodes production of a certain protein by the plant. In some embodiments, the trait of decreased LOX-2 and LOX-3 function is conferred to the plant by introducing a nucleotide sequence (e.g., using plant transformation methods) that encodes production of a certain protein by the plant. In other non-limiting embodiments, the trait of decreased FAD (e.g., FAD2B, FAD3C, FAD3D) function is conferred to the plant by introducing a nucleotide sequence (e.g., using plant transformation methods) that encodes production of a certain protein by the plant. In some embodiments, the trait of decreased FAD3C and LOX-2 function is conferred to the plant by introducing a nucleotide sequence (e.g., using plant transformation methods) that encodes production of a certain protein by the plant. In certain non-limiting embodiments, the desired trait is conferred to a plant by causing a null mutation in the plant's genome (e.g., when the desired trait is reduced expression or no expression of a certain trait). In certain non-limiting embodiments, the desired trait of decreased LOX-2 and/or LOX-3 function is conferred to a plant by crossing two plants to create offspring that express the desired trait. In some embodiments, the trait of decreased LOX-2 and LOX-3 function is conferred to the plant by crossing two plants to create offspring that express the desired trait(s). In other non-limiting embodiments, the trait of decreased FAD (e.g., FAD2B, FAD3C, FAD3D) function is conferred to the plant by crossing two plants to create offspring that express the desired trait(s). In some embodiments, the trait of decreased FAD3C and LOX-2 function is conferred to the plant by crossing two plants to create offspring that express the desired trait(s). It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

Mutating a LOX (e.g., LOX-2, LOX-3) and or FAD (e.g., FAD2B, FAD3C, FAD3D) gene by the methods of the present disclosure can comprise one or more insertions, substitutions, or deletions of about 1-23, 2-23, 3-23, 4-23, 5-23, 6-23, 7-23, 8-23, 9-23, or 10-23 (e.g., about 1, 2, 3, 4, 5, 6, 7, 8, 9, 10, 11, 12, 13, 14, 15, 16, 17, 18, 19, 20, 21, 22, or 23) nucleotides of the LOX and/FAD gene in the genome of a plant cell or plant part. In particular, methods of the present disclosure can comprise one or more insertions, substitutions, or deletions of about 11 nucleotides in one or more of a LOX-2 gene, a LOX-3 gene, a FAD2B gene, a FAD3C gene and/or a FAD3D gene. For example, mutating LOX-2 gene in a plant cell or plant part by the methods of the present disclosure can comprise genomically editing the plant by introducing a 11 nucleotide deletion in one or more of a LOX-2 gene, a LOX-3 gene, a FAD2B gene, a FAD3C gene and/or a FAD3D gene. The deletion can be an in-frame deletion or an out-of-frame deletion. In another example, mutating the one or more of a LOX-2 gene, a LOX-3 gene, a FAD2B gene, a FAD3C gene and/or a FAD3D gene. in a plant cell or plant part by the methods of the present disclosure can comprise an 8 nucleotide deletion in the one or more of a LOX-2 gene, a LOX-3 gene, a FAD2B gene, a FAD3C gene and/or a FAD3D gene. The deletion can be an in-frame deletion or an out-of-frame deletion.

Mutating a nucleic acid sequence encoding the one or more of a LOX-2 protein, a LOX-3 protein, a FAD2B protein, a FAD3C protein and/or a FAD3D protein by the methods of the present disclosure can comprise insertions, substitutions, or deletions in one or more of exons (e.g., exon 4, exon 4, exon 1, exon 2, and exon 3 of genes encoding LOX-2, LOX-3, FAD2B, FAD3C, and FAD3D proteins, respectively.) Mutation can comprise insertions, substitutions, or deletions in one or more of the introns of a LOX gene or in a regulatory element (e.g., promoter, 5′ untranslated region, and/or 3′ untranslated region) that regulates the expression of the LOX gene. In some instances, mutation by the methods of the present disclosure can comprise one or more insertions, substitutions, or deletions in a nucleotide region upstream of exon 8, exon 7, exon 6, or exon 5 of the LOX-2 gene. In some instances, mutation by the methods of the present disclosure can comprise one or more insertions, substitutions, or deletions, or part thereof at least partially in a nucleotide region of exon 4 of the LOX-3 gene. In particular, mutating LOX-2 gene in the genome of a plant cell or plant part by the methods of the present disclosure can comprise one or more insertions, substitutions, or deletions in a nucleotide region corresponding to exon 4 of the LOX-2 gene. For example, mutation of LOX-2 gene in a plant cell or plant part by the methods of the present disclosure can comprise deletion of about 1, 2, 3, 4, 5, 6, 7, 8, 9, 10, 11, 12, 13, 14, 15, 16, 17, 18, 19, 20, 21, 22, or 23 nucleotides in exon 4 of the LOX-2 gene, such as in exon 4 of the LOX-2 gene of Pisum sativum.

Mutating a nucleic acid sequence encoding the FAD2 and/or FAD3 protein by the methods of the present disclosure can comprise insertions, substitutions, or deletions in one or more of exon 1, exon 2, and exon 3 of genes encoding FAD2B, FAD3C, and FAD3D proteins, respectively. Mutation can comprise insertions, substitutions, or deletions in one or more of the introns of a FAD2 and/or FAD3 genes or in a regulatory element (e.g., promoter, 5′ untranslated region, and/or 3′ untranslated region) that regulates the expression of the FAD2 and/or FAD3 genes. In some embodiments, mutation by the methods of the present disclosure can comprise one or more insertions, substitutions, or deletions are at least partially in a nucleotide region of exon 1 of the FAD2B gene. In some embodiments, mutation by the methods of the present disclosure can comprise one or more insertions, substitutions, or deletions are at least partially in a nucleotide region of exon 2 of the FAD3C gene. In some embodiments, mutation by the methods of the present disclosure can comprise one or more insertions, substitutions, or deletions are at least partially in a nucleotide region of exon 3 of the FAD3D gene.

The one or more insertions, substitutions, or deletions in a LOX-2 gene can be in a nucleotide region that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 10. The one or more insertions, substitutions, or deletions in a LOX-3 gene can be in a nucleotide region that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO:27. The one or more insertions, substitutions, or deletions in a FAD2B gene can be in a nucleotide region that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 36. The one or more insertions, substitutions, or deletions in a FAD3C gene can be in a nucleotide region that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 46. The one or more insertions, substitutions, or deletions in a FAD3D gene can be in a nucleotide region that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 56.

For example, the one or more insertions, substitutions, or deletions in LOX-2 gene can comprise a deletion of about 1, 2, 3, 4, 5, 6, 7, 8, 9, 10, 11, 12, 13, 14, 15, 16, 17, 18, 19, 20, 21, 22, or 23 nucleotides in a nucleotide region that comprises the nucleic acid sequence of SEQ ID NO: 10. In particular, the one or more insertions, substitutions, or deletions in LOX-2 gene can comprise a deletion of about 11 nucleotides, such as deletion of nucleotide 1521 through 1531 of SEQ ID NO: 10. In another example, the one or more insertions, substitutions, or deletions in LOX-2 gene can comprise a deletion of about 8 nucleotides, such as deletion of nucleotide 1523 through 1530 of SEQ ID NO: 10. In some instances, exon 4 of LOX-2 gene of Pisum sativum is mutated by the methods of the present disclosure. Thus, the one or more insertions, substitutions, or deletions in LOX-2 gene can comprise a deletion of about 11 nucleotides in a nucleotide region that comprises the nucleic acid sequence of SEQ ID NO: 3 (exon 4 of Pea LOX-2). In particular, the one or more insertions, substitutions, or deletions in LOX-2 gene can comprise a deletion of nucleotide 284 through 294 of SEQ ID NO: 3. The one or more insertions, substitutions, or deletions in LOX-2 gene can comprise a deletion of about 8 nucleotides in a nucleotide region that comprises the nucleic acid sequence of SEQ ID NO: 3 (exon 4 of Pea LOX-2). In particular, the one or more insertions, substitutions, or deletions in LOX-2 gene can comprise a deletion of nucleotide 286 through 293 of SEQ ID NO: 3.

For example, the one or more insertions, substitutions, or deletions in LOX-3 gene can comprise a deletion of about 1, 2, 3, 4, 5, 6, 7, 8, 9, 10, 11, 12, 13, 14, 15, 16, 17, 18, 19, 20, 21, 22, 23, 24, 25, 26, 27, or 28 nucleotides in a nucleotide region that comprises the nucleic acid sequence of SEQ ID NO: 27. In particular, the one or more insertions, substitutions, or deletions in LOX-3 gene can comprise a deletion of about 28 nucleotides, such as deletion of nucleotide 1129 through 1156 of SEQ ID NO: 27. In some instances, exon 4 of LOX-3 gene is mutated by the methods of the present disclosure. Thus, the one or more insertions, substitutions, or deletions in LOX-3 gene can comprise a deletion of about 28 nucleotides in a nucleotide region that comprises the nucleic acid sequence of SEQ ID NOs: 22. In particular, the one or more insertions, substitutions, or deletions in LOX-3 gene can comprise a deletion of nucleotide 136 through 163 of SEQ ID NO: 22.

For example, the one or more insertions, substitutions, or deletions in FAD2B gene can comprise a deletion of about 1, 2, 3, 4, 5, 6, 7, 8, 9, 10, 11, 12, 13, 14, 15, 16, 17, 18, 19, 20, 21, 22, or 23 nucleotides in a nucleotide region that comprises the nucleic acid sequence of SEQ ID NO: 36. In particular, the one or more insertions, substitutions, or deletions in FAD2B gene can comprise a deletion of about 2-8 nucleotides, such as deletion of nucleotide 59 through 66 of SEQ ID NO: 36 or a deletion of nucleotide 60 through 61 of SEQ ID NO: 36. In some instances, exon 1 of FAD2B gene is mutated by the methods of the present disclosure. Thus, the one or more insertions, substitutions, or deletions in FAD2 gene can comprise a deletion of about 2-8 nucleotides in a nucleotide region that comprises the nucleic acid sequence of SEQ ID NO: 29. In particular, the one or more insertions, substitutions, or deletions in FAD2B gene can comprise a deletion of nucleotide 59 through 66 of SEQ ID NO: 29 or a deletion of nucleotide 60 through 61 of SEQ ID NO: 29.

For example, the one or more insertions, substitutions, or deletions in FAD3C gene can comprise a deletion of about 1, 2, 3, 4, 5, 6, 7, 8, 9, 10, 11, 12, 13, 14, 15, 16, 17, 18, 19, 20, 21, 22, 23, 24, 25, 30, 35, 40, 45, 46, 47, 48, or 49 nucleotides in a nucleotide region that comprises the nucleic acid sequence of SEQ ID NO: 46. In particular, the one or more insertions, substitutions, or deletions in FAD3C gene can comprise a deletion of about 8-49 nucleotides, such as a deletion of nucleotide 457 through 464 of SEQ ID NO: 46 or a deletion of nucleotide 416 through 464 of SEQ ID NO: 46. In some instances, exon 2 of FAD3C gene is mutated by the methods of the present disclosure. Thus, the one or more insertions, substitutions, or deletions in FAD3 gene can comprise a deletion of about 8-49 nucleotides in a nucleotide region that comprises the nucleic acid sequence of SEQ ID NO: 39. In particular, the one or more insertions, substitutions, or deletions in FAD3C gene can comprise a deletion of nucleotide 33 through 40 of SEQ ID NO: 39 or a deletion of nucleotide 1 through 44 of SEQ ID NO: 39.

For example, the one or more insertions, substitutions, or deletions in FAD3D gene can comprise a deletion of about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 101, 102, 103, 104, 105, 106, or 107 nucleotides in a nucleotide region that comprises the nucleic acid sequence of SEQ ID NO: 56. In particular, the one or more insertions, substitutions, or deletions in FAD3D gene can comprise a deletion of about 5-107 nucleotides, such as deletion of nucleotide 775 through 779 of SEQ ID NO: 56, or a deletion of nucleotide 745 through 851 of SEQ ID NO: 56. In some instances, exon 3 of FAD3D gene is mutated by the methods of the present disclosure. Thus, the one or more insertions, substitutions, or deletions in FAD3D gene can comprise a deletion of about 5-107 nucleotides in a nucleotide region that comprises the nucleic acid sequence of 49. In particular, the one or more insertions, substitutions, or deletions in FAD3D gene can comprise a deletion of nucleotide 55 through 59 of SEQ ID NO: 49 or a deletion of nucleotide 30 through 67 of SEQ ID NO: 49.

A LOX-2 gene mutated by the methods of the present disclosure (e.g., by deletion of about 11 nucleotides or 8 nucleotides in exon 4 of the LOX-2 gene) can encode a truncated LOX-2 protein. A truncated LOX-2 protein can have loss-of-function or reduced function, as compared to a full length LOX-2 protein, such as a protein encoded by WT LOX-2 gene (i.e., LOX-2 gene that has not been mutated). Truncated LOX-2 protein can be found in plants or plant parts that contain mutated LOX-2 gene. Truncated LOX-2 protein can also be found in plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant protein, plant concentrate (e.g., whole plant concentrate or plant part concentrate such as yellow pea protein concentrate), plant powder (e.g., formulated powder, such as formulated plant part powder (e.g., seed flour)), and plant biomass (e.g., dried biomass, such as crushed and/or powdered biomass) obtained from such plants with a mutated LOX-2 gene. In some embodiments, a truncated LOX-2 protein comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of SEQ ID NO: 8 or 9. In some embodiments, mutating LOX-2 gene of Pisum sativum can result in a truncated LOX-2 protein that comprises the amino acid sequence of SEQ ID NO: 8 or 9. Truncated LOX-2 protein from Pisum sativum can have loss-of-function or reduced function as compared to a full length Pisum sativum LOX-2 protein, such as a protein comprising the amino acid sequence of SEQ ID NO: 7.

Similarly, the LOX-3 gene mutated by the methods of the present disclosure (e.g., by deletion of about 11 nucleotides in exon 4 of the LOX-3 gene) can encode a truncated LOX-3 protein. A truncated LOX-3 protein can have loss-of-function or reduced function, as compared to a full length LOX-3 protein, such as a protein encoded by WT LOX-3 gene (i.e., LOX-3 gene that has not been mutated). Truncated LOX-3 protein can be found in plants or plant parts that contain mutated LOX-3 gene. Truncated LOX-3 protein can also be found in plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant protein, plant concentrate (e.g., whole plant concentrate or plant part concentrate such as yellow pea protein concentrate), plant powder (e.g., formulated powder, such as formulated plant part powder (e.g., seed flour)), and plant biomass (e.g., dried biomass, such as crushed and/or powdered biomass) obtained from such plants with a mutated LOX-3 gene. In some embodiments, a truncated LOX-3 protein comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of SEQ ID NO: 26. In some embodiments, mutating LOX-3 gene of Pisum sativum can result in a truncated LOX-3 protein that comprises the amino acid sequence of SEQ ID NO: 26. Truncated LOX-3 protein from Pisum sativum can have loss-of-function or reduced function as compared to a full length Pisum sativum LOX-3 protein, such as a protein comprising the amino acid sequence of SEQ ID NO: 25.

A FAD2B gene mutated by the methods of the present disclosure (e.g., by deletion of about 11 nucleotides in exon x of the FAD2 gene) can encode a truncated FAD2B protein. A truncated FAD2B protein can have loss-of-function or reduced function, as compared to a full length FAD2B protein, such as a protein encoded by WT FAD2B gene (i.e., FAD2B gene that has not been mutated). Truncated FAD2B protein can be found in plants or plant parts that contain mutated FAD2B gene. Truncated FAD2B protein can also be found in plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant protein, plant concentrate (e.g., whole plant concentrate or plant part concentrate such as yellow pea protein concentrate), plant powder (e.g., formulated powder, such as formulated plant part powder (e.g., seed flour)), and plant biomass (e.g., dried biomass, such as crushed and/or powdered biomass) obtained from such plants with a mutated FAD2B gene. In some embodiments, a truncated FAD2B protein comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of SEQ ID NO: 34 or 35. In some embodiments, mutating FAD2 gene of Pisum sativum can result in a truncated FAD2B protein that comprises the amino acid sequence of SEQ ID NO: 34 or 35. Truncated FAD2 protein from Pisum sativum can have loss-of-function or reduced function as compared to a full length Pisum sativum FAD2B protein, such as a protein comprising the amino acid sequence of SEQ ID NO: 33.

A FAD3C gene mutated by the methods of the present disclosure (e.g., by deletion of about 11 nucleotides in exon 2 of the FAD3C gene) can encode a truncated FAD3C protein. A truncated FAD3C protein can have loss-of-function or reduced function, as compared to a full length FAD3C protein, such as a protein encoded by WT FAD3C gene (i.e., FAD3C gene that has not been mutated). Truncated FAD3C protein can be found in plants or plant parts that contain mutated FAD3C gene. Truncated FAD3C protein can also be found in plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant protein, plant concentrate (e.g., whole plant concentrate or plant part concentrate such as yellow pea protein concentrate), plant powder (e.g., formulated powder, such as formulated plant part powder (e.g., seed flour)), and plant biomass (e.g., dried biomass, such as crushed and/or powdered biomass) obtained from such plants with a mutated FAD3C gene. In some embodiments, a truncated FAD3C protein comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of SEQ ID NOs: 44 or 45. In some embodiments, mutating FAD3C gene of Pisum sativum can result in a truncated FAD3C protein that comprises the amino acid sequence of SEQ ID NOs: 44 or 45. Truncated FAD3C protein from Pisum sativum can have loss-of-function or reduced function as compared to a full length Pisum sativum FAD3C protein, such as a protein comprising the amino acid sequence of SEQ ID NO: 43.

A FAD3D gene mutated by the methods of the present disclosure (e.g., by deletion of about 11 nucleotides in exon 3 of the FAD3D gene) can encode a truncated FAD3D protein. A truncated FAD3D protein can have loss-of-function or reduced function, as compared to a full length FAD3D protein, such as a protein encoded by WT FAD3D gene (i.e., FAD3D gene that has not been mutated). Truncated FAD3D protein can be found in plants or plant parts that contain mutated FAD3D gene. Truncated FAD3D protein can also be found in plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant protein, plant concentrate (e.g., whole plant concentrate or plant part concentrate such as yellow pea protein concentrate), plant powder (e.g., formulated powder, such as formulated plant part powder (e.g., seed flour)), and plant biomass (e.g., dried biomass, such as crushed and/or powdered biomass) obtained from such plants with a mutated FAD3D gene. In some embodiments, a truncated FAD3D protein comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of SEQ ID NOs: 54 or 55. In some embodiments, mutating FAD3D gene of Pisum sativum can result in a truncated FAD3D protein that comprises the amino acid sequence of SEQ ID NO: 54 or 55. Truncated FAD3D protein from Pisum sativum can have loss-of-function or reduced function as compared to a full length Pisum sativum FAD3D protein, such as a protein comprising the amino acid sequence of SEQ ID NO: 53.

(i) Methods of Introducing Mutations using RNA-Guided Endonucleases

Mutating a LOX (e.g., LOX-2, LOX-3) and/or FAD (e.g., FAD2B, FAD3C, FAD3D) genes by the methods of the present disclosure can comprise cleaving the genome of the plant by a non-naturally occurring heterologous CRISPR-Cas genomic editing system. In some embodiments, the one or more insertions, substitutions, or deletions in a gene encoding a LOX protein or a FAD protein are introduced following cleavage of one or more genes selected from the group consisting of LOX-2, LOX-3, FAD2, and FAD3 by a nuclease that is part of a Type II or Type V CRISPR system. In some embodiments, the endonuclease that is part of a Type II or Type V CRISPR system is a Cas9 nuclease, a Cpf1 (Cas12a) nuclease, or a Cms1 nuclease.

In many embodiments of the methods described herein, the heterologous CRISPR-Cas genomic editing system is a CRISPR-Cas12a genomic editing system, comprising or encoding a Cas12a endonuclease or at least one Cas12a ortholog endonuclease selected from the group consisting of Lb5Cas12a, CMaCas12a, BsCas12a, BoCas12a, MlCas12a, Mb2Cas12a, TsCas12a, and MAD7 endonucleases. In some embodiments, the endonuclease comprises an amino acid sequence having at least 80% sequence identity to the amino acid sequence set forth in SEQ ID NO: 16 and retains endonuclease activity.

The CRISPR-Cas12a genomic editing system may comprise at least one guide RNA (gRNA) operatively arranged with the ortholog endonuclease for genomic editing of a target DNA binding the gRNA. In embodiments, the system may comprise a CRISPR-Cas12a expression system encoding the Cas12a ortholog nucleases and crRNAs for forming gRNAs that are coactive with the Cas12a nucleases.

Also described herein are expression constructs that can be used in a method for mutating LOX and or FAD genes in a plant cell or plant part. A recombinant DNA construct of the present disclosure may contain a guide RNA (gRNA) cassette to drive mutation of the LOX, FAD genes. For example, a recombinant DNA construct of the present disclosure may contain a gRNA cassette to drive a deletion (e.g., 11 nucleotide deletion) at exon 4 of LOX-2 gene. The gRNA can be specific to a region of exon 4 of LOX-2 gene. For example, the gRNA can be specific to a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 3. In particular instances, the gRNA can facilitate binding of an RNA guided nuclease that cleaves an exonic region of LOX-2, LOX-3, FAD2 or FAD3 gene of Pisum sativum and causes non-homologous end joining to introduce a mutation at the cleavage site. In some instances, a gRNA may comprise a targeting region that is complementary to a targeted sequence as well as another region that allows the gRNA to form a complex with a nuclease (e.g., a CRISPR nuclease) of interest. The targeting region (i.e. spacer) of a gRNA that binds to the region of the LOX-2, LOX-3, FAD2 or FAD3 gene for use in the method described herein above can be about 100-300 nucleotides long with the targeting region therein about 10-40 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long). For example, the targeting region of a gRNA for use in the method described hereinabove can be 24 nucleotides in length. In some embodiments, the targeting region of a gRNA comprises a nucleic acid sequence having at least 75% sequence identity to the nucleic acid sequence of the target regions in the LOX-2, LOX-3, FAD2B, FAD3C, or FAD3D gene. In some embodiments, the targeting region of a gRNA for use in the method described herein comprises the nucleic acid sequence that shares at least 75% sequence identity with SEQ ID NO: 4, 23, 30, 40, or 50. In particular instances, the targeting region of a gRNA for use in the method described herein is encoded by a nucleic acid sequence comprising the nucleic acid sequence of SEQ ID NO: 4, 23, 30, 40, or 50.

A number of promoters may be used in the practice of the disclosure. The promoter may have a constitutive expression profile. Constitutive promoters include the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like.

Alternatively, promoters for use in the methods of the present disclosure can be tissue-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Alternatively, promoters for use in the methods of the present disclosure can be developmentally-regulated promoters. Such promoters may show a peak in expression at a particular developmental stage. Such promoters have been described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like.

Alternatively, promoters for use in the methods of the present disclosure can be promoters that are induced following the application of a particular biotic and/or abiotic stress. Such promoters have been described in the art, e.g., Yi et al. (2010) Planta 232: 743-754; Yamaguchi-Shinozaki and Shinozaki (1993) Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

Alternatively, promoters for use in the methods of the present disclosure can be cell-preferred promoters. Such promoters may preferentially drive the expression of a downstream gene in a particular cell type such as a mesophyll or a bundle sheath cell. Such cell-preferred promoters have been described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like.

It is recognized that a specific, non-constitutive expression profile may provide an improved plant phenotype relative to constitutive expression of a gene or genes of interest. For instance, many plant genes are regulated by light conditions, the application of particular stresses, the circadian cycle, or the stage of a plant's development. These expression profiles may be important for the function of the gene or gene product in planta. One strategy that may be used to provide a desired expression profile is the use of synthetic promoters containing cis-regulatory elements that drive the desired expression levels at the desired time and place in the plant. Cis-regulatory elements that can be used to alter gene expression in planta have been described in the scientific literature (Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

A recombinant DNA construct described herein may contain transfer DNA (T-DNA) sequences. For example, a recombinant DNA construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a gRNA of the present disclosure into nuclear DNA genome of a host plant (e.g., a pea plant).

Also described herein is a bacterium containing a recombinant DNA construct of the present disclosure. For example, the present disclosure may provide an Agrobacterium tumefaciens containing a recombinant DNA construct that comprises a gRNA to drive mutation of LOX-2 gene.

Also described herein is a plasmid containing a recombinant DNA construct of the present disclosure. For example, the present disclosure may provide a plasmid containing a recombinant DNA construct that comprises a gRNA to drive mutation of LOX-2, LOX-3, FAD2 or FAD3 gene.

Also described herein is a recombinant virus containing a recombinant DNA construct of the present disclosure. For example, the present disclosure may provide a recombinant virus containing a recombinant DNA construct that comprises a gRNA, wherein the gRNA can drive mutation of LOX-2 gene. A recombinant virus described herein can be a recombinant lentivirus, a recombinant retrovirus, a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

In some embodiments, a recombinant DNA construct described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

Reporter genes or selectable marker genes may also be included in the expression cassettes of the present invention. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other polynucleotides that could be employed on the expression constructs disclosed herein include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

Also disclosed herein are vectors encoded by recombinant DNA constructs. As used herein, “vector” refers to a DNA molecule such as a plasmid, cosmid, or bacterial phage for introducing a nucleotide construct, for example, a recombinant DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance. Provided herein are expression cassettes comprising gRNA sequence specific for exon 4 of LOX-2 gene located on a vector.

In some embodiments, the CRISPR-Cas12a system of the methods described herein may comprise one or vectors comprising at least one CRISPR RNA (crRNA) regulatory element operably linked to at least one nucleotide sequence encoding a CRISPR-Cas12a system crRNA for producing gRNA for targeting a target sequence, and at least one regulatory element, which may be the same as the crRNA regulatory element, or different therefrom, operably linked to a nucleotide sequence encoding the Cas12a ortholog endonuclease, for generation of a CRISPR-Cas12a editing structure by which the gRNA targets the target sequence and the Cas12a ortholog endonuclease cleaves a target DNA to alter gene expression in the cell, and wherein the CRISPR-associated nuclease, and the gRNA, do not naturally occur together. In such system, the at least one crRNA regulatory element may comprise one or more than one RNA polymerase II (Pol II) promoter, or alternatively, a single transcript unit (STU) regulatory element, or one or more promoter(s) selected from the group consisting of ZmUbi, OsU6, OsU3, and U6 promoters.

In many embodiments of the methods described herein, genomically editing a plant, comprising introducing into such plant a non-naturally occurring heterologous CRISPR-Cas12a genomic editing system of a type as variously described hereinabove, to cause the Cas12a ortholog nuclease to edit DNA of LOX-2, LOX-3, FAD2 and/or FAD3 in the Pisum sativum plant to alter the plant's expression of LOX-2, LOX-3, FAD2 and/or FAD3 genes. The genomically editing may be performed so that the CRISPR-Cas12a genomic editing system targets PAM sites such as TTN, TTV, TTTV, NTTV, TATV, TATG, TATA, YTTN, GTTA, and/or GTTC.

Such method may be carried out at moderate temperatures, e.g., below 25° C. and above temperature producing freezing or frost damage of the plant. The editing method of the disclosure may be performed on a wide variety of plants. In particular application to Pisum sativum plant, the editing method may be carried out to edit the Pisum sativum plant at one or more of LOX-2, LOX-3, FAD2, and FAD3 genes thereof.

The CRISPR-Cas12a nuclease systems comprise the Cas12a ortholog endonucleases of the present disclosure (Lb5Cas12a, CMaCas12a, BsCas12a, BoCas12a, MlCas12a, Mb2Cas12a, MbCas12a, TsCas12a, and MAD7) and guide RNA. Expression systems for such CRISPR-Cas12a nuclease systems may readily be prepared in accordance with the present disclosure, encoding the Cas12a nucleases and crRNAs for forming gRNAs that are coactive with the Cas12a nucleases. The CRISPR-Cas12a nuclease systems may comprise constructs, e.g., complexes or otherwise operatively coupled structures, comprising any of such Cas12a ortholog endonucleases with corresponding guide RNA targeting a target sequence in a plant, so that the guide RNA targets the target sequence and the Cas12a ortholog endonuclease cleaves DNA in the Pisum sativum plant at one or more of LOX-2, LOX-3, FAD2, and FAD3 genes thereof, to alter gene expression.

(ii) RNA Interference

Function of LOX (e.g., LOX-2, LOX-3) or FAD (e.g., FAD2B, FAD3C, FAD3D) protein in a plant or plant part can be reduced by inhibiting or silencing the expression of LOX and/or FAD gene. Methods of the present disclosure can inhibit expression of LOX and/or FAD gene in a plant or plant part by RNA interference (RNAi). RNA interference is a biological process in which double-stranded RNA (dsRNA) molecules are involved in sequence-specific suppression of gene expression through translation or transcriptional repression. Two types of small RNA molecules—microRNA (miRNA) and small interfering RNA (siRNA)—are central to RNA interference. RNAs are the direct products of genes, and these small RNAs can direct enzyme complexes to degrade messenger RNA (mRNA) molecules and thus decrease their activity by preventing translation, via post-transcriptional gene silencing. Moreover, transcription can be inhibited via the pre-transcriptional silencing mechanism of RNA interference, through which an enzyme complex catalyzes DNA methylation at genomic positions complementary to complexed siRNA or miRNA.

Provided herein are methods for suppressing the expression of a LOX (e.g., LOX-2, LOX-3) or FAD (e.g., FAD2B, FAD3C, FAD3D) gene by using siRNA and/or miRNA molecules that are directed to the LOX gene, LOX RNA transcript, FAD gene or FAD mRNA transcript. In particular, methods of the present disclosure can inhibit or silence one or more of LOX-2, LOX-3, FAD2, and FAD3 genes in the genome of cells or parts of a plant by RNA interference, using siRNA and/or miRNA molecules that are directed to the one or more of LOX-2, LOX-3, FAD2, and FAD3 genes.

siRNA and/or miRNA molecules for use in the present methods can be complementary to about 1-23, 2-23, 3-23, 4-23, 5-23, 6-23, 7-23, 8-23, 9-23, or 10-23 (e.g., about 1, 2, 3, 4, 5, 6, 7, 8, 9, 10, 11, 12, 13, 14, 15, 16, 17, 18, 19, 20, 21, 22, or 23) nucleotides of one or more of LOX-2, LOX-3, FAD2, and FAD3 genes or the corresponding RNA transcripts. In particular, such siRNA and/or miRNA molecules can be complementary to a nucleotide region in exon 1, exon 2, exon 3, exon 4, exon 5, exon 6, exon 7, or exon 8 of LOX-2 gene. In some instances, the siRNA and/or miRNA molecules can be complementary to a nucleotide region that is upstream of exon 8, exon 7, exon 6, or exon 5 of the LOX-2 gene. In particular, the siRNA and/or miRNA molecules can be complementary to a nucleotide region corresponding to exon 4 of the LOX-2 gene. For example, the siRNA and/or miRNA molecules can be complementary to a nucleotide region corresponding to exon 4 of the LOX-2 gene of Pisum sativum. In some embodiments, such siRNA and/or miRNA molecules can be complementary to a nucleotide region in exon 4 of LOX-3 gene, exon 1 of FAD2B gene, exon 2 of FAD3C gene, or exon 3 of FAD3D gene. In some instances, the siRNA and/or miRNA molecules can be complementary to a nucleotide region that corresponds to or encompasses exon 4 of LOX-3 gene, exon 1 of FAD2B gene, exon 2 of FAD3C gene, or exon 3 of FAD3D gene.

In some embodiments, the siRNA and/or miRNA molecules can be complementary to a nucleotide region that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of any one of SEQ

ID NOs: 10, 27, 36, 46, and 56. For example, the siRNA and/or miRNA molecules can be complementary to a nucleotide region that comprises the nucleic acid sequence of any one of SEQ ID NOs: 10, 27, 36, 46, and 56. In some instances, the LOX-2 gene of Pisum sativum is silenced by the RNA interference methods of the present disclosure that target exon 4 of the LOX-2 gene. Thus, the siRNA and/or miRNA molecules can be complementary to a nucleotide region that comprises the nucleic acid sequence of SEQ ID NO: 3. In some embodiments, one or more of LOX-3, FAD2 and FAD3 genes of Pisum sativum is silenced by the RNA interference methods of the present disclosure.

(iii) Modification of Transcriptional Regulation

Function and/or expression of a LOX (e.g., LOX-2, LOX-3) or FAD (e.g., FAD2B, FAD3C,

FAD3D) protein in a plant or plant part can be reduced by inhibiting or silencing the expression of LOX and/or FAD gene. Methods of the present disclosure can inhibit expression of LOX and/or FAD gene in a plant or plant part by inactivation of the promoter sequence of the gene.

The promoter sequence of one or more of LOX-2, LOX-3, FAD2, and FAD3 genes can be inactivated by insertion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. For example, the activity of the promoter sequence of LOX-2 gene can be reduced or decreased by insertion of one or more nucleotides.

Additionally or alternatively, the promoter sequence of one or more of LOX-2, LOX-3, FAD2, and FAD3 genes can be inactivated by deletion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. For example, the activity of the promoter sequence of LOX-2 gene can be reduced or decreased by deletion of one or more nucleotides.

The promoter sequence of LOX-2, LOX-3, FAD2, and FAD3 genes can also be inactivated by replacement of the promoter sequence with one or more substitutes. In particular, the substitute can be a cisgenic substitute. For example, the promoter sequence of the LOX-2 gene can be inactivated by replacement of the promoter sequence with one or more cisgenic substitutes. Alternatively, the substitute can be a transgenic substitute. For example, the promoter sequence of LOX-2 gene can be inactivated by replacement of the promoter sequence with one or more transgenic substitutes.

In some instances, the promoter sequence of one or more of LOX-2, LOX-3, FAD2, and FAD3 genes is inactivated by correction of the promoter sequence. A promoter sequence may be corrected by deletion, modification, and/or correction of one or more polymorphisms or mutations that would otherwise enhance the activity of the promoter sequence. In particular, the promoter sequence of one or more of LOX-2, LOX-3, FAD2, and FAD3 genes can be inactivated by: (i) detection of one or more polymorphism or mutation that enhances the activity of the promoter sequence; and (ii) correction of the promoter sequences by deletion, modification, and/or correction of the polymorphism or mutation.

In some instances, the promoter sequence of one or more of LOX-2, LOX-3, FAD2, and FAD3 genes is inactivated by insertion, deletion, and/or modification of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) upstream nucleotide sequences. In particular, for example, the activity of the promoter sequence of LOX-2 gene can be reduced or decreased by insertion, deletion, and/or modification of one or more upstream nucleotide sequences.

In some instances, the promoter sequence of one or more of LOX-2, LOX-3, FAD2, and FAD3 genes are inactivated by addition, insertion, and/or engineering of cis-acting factors. For example, the activity of the promoter sequence of one or more LOX-2, LOX-3, FAD2, and FAD3 genes can be reduced or decreased by addition, insertion, and/or engineering of cis-acting factors that interact with and modify the promoter sequence.

Function and/or expression of the one or more of LOX-2, LOX-3, FAD2, and FAD3 genes can also be decreased or inhibited by modulation (e.g., increase or decrease) of expression of one or more transcription factor genes. For example, modulation of expression of the one or more transcription factor genes inactivates promoter sequence of the one or more of LOX-2, LOX-3, FAD2, and FAD3 genes. For example, modulation of expression of the one or more transcription factor genes can inhibit expression of the one or more of LOX-2, LOX-3, FAD2, and FAD3 genes.

Function and/or expression of the one or more of LOX-2, LOX-3, FAD2, and FAD3 genes can also be decreased by insertion, modification, and/or engineering of transcription factor binding sites or enhancer elements. For example, inhibition of the one or more of LOX-2, LOX-3, FAD2, and FAD3 gene expression and/or function encompasses insertion of novel transcription factor binding sites or enhancer elements. Alternatively, inhibition of the one or more of LOX-2, LOX-3, FAD2, and FAD3 gene expression and/or function can encompass modification and/or engineering of existing transcription factor binding sites or enhancer elements.

(iv) Insertion of Negative Regulatory Sequence

Function and/or expression of the one or more of LOX-2, LOX-3, FAD2, and FAD3 genes can also be decreased or inhibited by insertion of one or more negative regulatory sequences of the gene. For example, to inhibit the expression and/or function of the LOX-2 gene, a part or whole of one or more negative regulatory sequences of the LOX-2 gene can be inserted in the genome of a plant cell or plant part. The negative regulatory sequence of the gene can be in a cis location. Alternatively, the negative regulatory sequence of the gene may be in a trans location. Negative regulatory sequences of the one or more of LOX-2, LOX-3, FAD2, and FAD3 genes can also include upstream open reading frames (uORFs). In some instances, a negative regulatory sequence can be inserted in a region upstream of the any one or more of LOX-2, LOX-3, FAD2, and FAD3 gene in order to inhibit the expression and/or function of the gene.

(v) Recombinant DNA Construct

Also described herein are expression constructs that can be used in a method for mutating one or more of LOX-2, LOX-3, FAD2, and FAD3 genes in a plant cell or plant part. A recombinant DNA construct of the present disclosure may contain a guide RNA (gRNA) cassette to drive mutation of the one or more of LOX-2, LOX-3, FAD2, and FAD3 genes. For example, a recombinant DNA construct of the present disclosure may contain a gRNA cassette to drive a deletion (e.g., 11 nucleotide deletion or 8 nucleotide deletion) at exon 4 of LOX-2 gene. The gRNA can be specific to a region of exon 4 of LOX-2 gene. For example, the gRNA can be specific to a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 3. For example, the gRNA can be specific to a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 22. For example, the gRNA can be specific to a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 29. For example, the gRNA can be specific to a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 39. For example, the gRNA can be specific to a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 49. In particular instances, the gRNA can facilitate binding of an RNA guided nuclease that cleaves a region of exon 4 of LOX-2 gene of Pisum sativum and causes non-homologous end joining to introduce a mutation at the cleavage site. In some instances, a gRNA may comprise a targeting region that is complementary to a targeted sequence as well as another region that allows the gRNA to form a complex with a nuclease (e.g., a CRISPR nuclease) of interest. The targeting region (i.e. spacer) of a gRNA that binds to the region of the LOX-2 gene for use in the method described herein can be 10-40 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long). For example, the targeting region of a gRNA for use in the method described hereinabove can be 24 nucleotides in length. In some embodiments, the targeting region of a gRNA is encoded by a nucleic acid sequence comprising a sequence having at least 75% sequence identity to the nucleic acid sequence of SEQ ID NO: 4. In some embodiments, the targeting region of a gRNA comprises a nucleic acid sequence having at least 75% sequence identity to the nucleic acid sequence of SEQ ID NO: 23. In some embodiments, the targeting region of a gRNA is encoded by a nucleic acid sequence comprising a nucleic acid sequence having at least 75% sequence identity to the nucleic acid sequence of SEQ ID NO: 30. In some embodiments, the targeting region of a gRNA is encoded by a nucleic acid sequence comprising a nucleic acid sequence having at least 75% sequence identity to the nucleic acid sequence of SEQ ID NO: 40. In some embodiments, the targeting region of a gRNA is encoded by a nucleic acid sequence comprising a nucleic acid sequence having at least 75% sequence identity to the nucleic acid sequence of SEQ ID NO: 50. In particular instances, the targeting region of a gRNA for use in the method described herein is encoded by a nucleic acid sequence comprising the nucleic acid sequence of SEQ ID NO: 4.

A number of promoters may be used in the practice of the disclosure. The promoter may have a constitutive expression profile. Constitutive promoters include the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like.

Alternatively, promoters for use in the methods of the present disclosure can be tissue-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Alternatively, promoters for use in the methods of the present disclosure can be developmentally-regulated promoters. Such promoters may show a peak in expression at a particular developmental stage. Such promoters have been described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like.

Alternatively, promoters for use in the methods of the present disclosure can be promoters that are induced following the application of a particular biotic and/or abiotic stress. Such promoters have been described in the art, e.g., Yi et al. (2010) Planta 232: 743-754; Yamaguchi-Shinozaki and Shinozaki (1993) Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

Alternatively, promoters for use in the methods of the present disclosure can be cell-preferred promoters. Such promoters may preferentially drive the expression of a downstream gene in a particular cell type such as a mesophyll or a bundle sheath cell. Such cell-preferred promoters have been described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like.

It is recognized that a specific, non-constitutive expression profile may provide an improved plant phenotype relative to constitutive expression of a gene or genes of interest. For instance, many plant genes are regulated by light conditions, the application of particular stresses, the circadian cycle, or the stage of a plant's development. These expression profiles may be important for the function of the gene or gene product in planta. One strategy that may be used to provide a desired expression profile is the use of synthetic promoters containing cis-regulatory elements that drive the desired expression levels at the desired time and place in the plant. Cis-regulatory elements that can be used to alter gene expression in planta have been described in the scientific literature (Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

A recombinant DNA construct described herein may contain transfer DNA (T-DNA) sequences. For example, a recombinant DNA construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a gRNA of the present disclosure into nuclear DNA genome of a host plant (e.g., a pea plant).

Also described herein is a bacterium containing a recombinant DNA construct of the present disclosure. For example, the present disclosure may provide an Agrobacterium tumefaciens containing a recombinant DNA construct that comprises a gRNA to drive mutation of any one of LOX-2, LOX-3, FAD2, and FAD3 genes.

Also described herein is a plasmid containing a recombinant DNA construct of the present disclosure. For example, the present disclosure may provide a plasmid containing a recombinant DNA construct that comprises a gRNA to drive mutation of any one of LOX-2, LOX-3, FAD2, and FAD3 genes.

Also described herein is a recombinant virus containing a recombinant DNA construct of the present disclosure. For example, the present disclosure may provide a recombinant virus containing a recombinant DNA construct that comprises a gRNA, wherein the gRNA can drive mutation of any one of LOX-2, LOX-3, FAD2, and FAD3 genes. A recombinant virus described herein can be a recombinant lentivirus, a recombinant retrovirus, a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

In some embodiments, a recombinant DNA construct described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

Reporter genes or selectable marker genes may also be included in the expression cassettes of the present invention. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other polynucleotides that could be employed on the expression constructs disclosed herein include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

Also disclosed herein are vectors encoded by recombinant DNA constructs. As used herein, “vector” refers to a DNA molecule such as a plasmid, cosmid, or bacterial phage for introducing a nucleotide construct, for example, a recombinant DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance. Provided herein are expression cassettes comprising gRNA sequence specific for exon 4 of LOX-2 gene located on a vector. Also provided herein are expression cassettes comprising gRNA sequence specific for exon x of LOX-3 gene located on a vector. Also provided herein are expression cassettes comprising gRNA sequence specific for exon x of FAD2B gene located on a vector. Also provided herein are expression cassettes comprising gRNA sequence specific for exon x of FAD3C gene located on a vector. Also provided herein are expression cassettes comprising gRNA sequence specific for exon x of FAD3D gene located on a vector.

### 2.7. Transformation of Plants

Disclosed herein are plants, plant cells, plant tissues, plant parts or seeds containing a mutation in a LOX (e.g., LOX-2, LOX-3) and/or FAD (e.g., FAD2B, FAD3C, FAD3D) gene. Also disclosed are control, or unmodified plants, plant cells, plant tissues, plant parts or seeds refer to plants, plant cells, plant tissues, plant parts or seeds that do not contain mutation in LOX (e.g., LOX-2, LOX-3) and/or FAD (e.g., FAD2B, FAD3C, FAD3D) gene and/or contain WT LOX (e.g., LOX-2, LOX-3) and/or WT FAD (e.g., FAD2B, FAD3C, FAD3D) gene. In certain instances, LOX and/or FAD gene is mutated by “transforming” plants, plant cells, plant tissues, plant parts or seeds with polynucleotides, such as polynucleotides described hereinabove. It is recognized that other exogenous or endogenous nucleic acid sequences or DNA fragments may also be incorporated into the plant cell. Agrobacterium-and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, Agrobacterium and viral mediated (Caulimoriviruses, Geminiviruses, RNA plant viruses), liposome mediated and the like.

While the present disclosure is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

For purpose of the present disclosure, the transformation can be “stable transformation”, wherein the transformation construct (e.g., a construct comprising a gRNA) is introduced into a host (e.g., a host plant, plant part, plant cell, etc.) and integrates into the genome of the host and is capable of being inherited by the progeny thereof; or “transient transformation”, wherein the transformation construct (e.g., a construct comprising a gRNA) is introduced into a host (e.g., a host plant, plant part, plant cell, etc.) and expressed temporally.

Also disclosed are plants comprising mutations in variants and fragments of the LOX-2, LOX-3, FAD2B, FAD3C and FAD3D sequences of the present disclosure. Such sequences include sequences that are orthologs of the disclosed LOX-2, LOX-3, FAD2B, FAD3C and FAD3D sequences. “Orthologs” is intended to mean genes derived from a common ancestral gene and which are found in different species as a result of speciation. Genes found in different species are considered orthologs when their nucleotide sequences and/or their encoded protein sequences share at least 75%, 80%, 85%, 90%, 91%, 92%, 93%, 94%, 95%, 96%, 97%, 98%, 99%, or greater sequence identity. Functions of orthologs are often highly conserved among species. Thus, plants comprising mutations in any gene encoding a LOX-2, LOX-3, FAD2B, FAD3C and FAD3D protein and at least 75% sequence identity to the sequences disclosed herein, or to variants or fragments thereof, are encompassed by the present disclosure.

Variant sequences can be isolated by PCR. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

Variant sequences may also be identified by analysis of existing databases of sequenced genomes. In this manner, corresponding sequences encoding LOX-2, LOX-3, FAD2B, FAD3C and

FAD3D protein can be identified and used in the methods of the present disclosure. The variant sequences will retain the biological activity of a LOX-2 protein (i.e., LOX activity), LOX-3 (i.e., LOX activity), FAD2B (i.e., FAD2 activity), FAD3C (i.e., FAD3 activity) and FAD3D (i.e., FAD3 activity). The present disclosure shows that, unexpectedly, a truncated LOX-2, LOX-3, FAD2B, FAD3C and/or FAD3D protein with loss-of-function or reduced function can lead to improved flavor in a plant, such as a plant that has been genetically-modified to edit one or more of the LOX-2, LOX-3, FAD2B, FAD3C and FAD3D genes.

Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

As indicated, gRNA (e.g., gRNA with targeting region specific to exon 4 of LOX-2 gene) can be used in recombinant DNA constructs and CRISPR nucleases, such as nucleases that are a part of a Type V or Type II CRISPR system, to transform plants of interest. Transformation protocols as well as protocols for introducing polypeptides or polynucleotide sequences into plants may vary depending on the type of plant or plant cell, i.e., monocot or dicot, targeted for transformation. The term “transform” or “transformation” refers to any method used to introduce polypeptides or polynucleotides into plant cells. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D′Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. In this manner, the present disclosure provides transformed seed (also referred to as “transgenic seed”) having a polynucleotide of the disclosure, for example, a recombinant DNA construct of the disclosure, stably incorporated into their genome.

The present disclosure may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), vegetables, ornamentals, and conifers. In some embodiments, the plant is selected from pea (Pisum sativum), bean (Phaseolus spp.), soybean (Glycine max), chickpea (Cicer arietinum), peanut (Arachis hypogaea), lentils (Lens culinaris, Lens esculenta), lupins (Lupinus spp.), mesquite (Prosopis spp.), carob (Ceratonia siliqua), tamarind (Tamarindus indica), alfalfa (Medicago sativa), and clover (Trifolium spp.).

Now that it has been demonstrated that mutation in LOX-2 gene (e.g., exon 4 of LOX-2 gene) and mutations in one or more of LOX-3, FAD2B, FAD3C and FAD3D genes can improve the flavor of plant, plant parts and plant products, other methods for mutating one or more of LOX-2, LOX-3, FAD2B, FAD3C and FAD3D genes in a plant of interest can be used. For example, a Cas (e.g., Cas9) endonuclease coupled with a guide RNA (gRNA) designed against the genomic sequence of interest (i.e., exon 4 of LOX-2 gene) can be introduced into the plant to effect a mutation in the LOX-2 gene. Alternatively, a Cpf1 (Cas12a) endonuclease coupled with a gRNA designed against the genomic sequence of interest, or a Cms1 endonuclease coupled with a gRNA designed against the genomic sequence of interest can be introduced into the plant to effect a mutation in the one or more of LOX-2, LOX-3, FAD2B, FAD3C and FAD3D genes. Other nuclease systems for use with the methods of the present invention include CRISPR systems (e.g., Type I, Type II, Type III, Type IV, and/or Type V CRISPR systems (Makarova et al 2020 Nat Rev Microbiol 18:67-83)) with their corresponding gRNA(s), TALENs, zinc finger nucleases (ZFNs), meganucleases, and the like. Alternatively, a deactivated CRISPR nuclease (e.g., a deactivated Cas9, Cpf1, or Cms1 endonuclease) fused to a transcriptional regulatory element can be targeted to a genomic location near the transcription start site for a gene encoding any one of LOX-2, LOX-3, FAD2B, FAD3C and FAD3D, thereby reducing the expression of the corresponding full-length protein encoded by the gene. Nucleases that are a part of a CRISPR system (i.e., CRISPR nucleases) can be introduced into a plant cell as an active protein or a nucleic acid can be introduced into a cell that encodes for the CRISPR nuclease. In specific embodiments, DNA or RNA can be introduced on an expression cassette in order to introduce a functional CRISPR nuclease to the target cell. When the functional CRISPR nuclease is present in the cell, it binds to the gRNA in order to cleave the target region of the one or more of LOX-2, LOX-3, FAD2B, FAD3C and FAD3D genes.

Modulation of the expression of a LOX (e.g., LOX-2, LOX-3) and/or FAD (e.g., FAD2B, FAD3C, FAD3D) protein-encoding gene may be achieved through the use of precise genome-editing technologies to modulate the expression of the endogenous sequence. In this manner, a cleavage site can be created that is repaired by non-homologous end joining to produce a mutation in the endogenous sequence. Alternatively, nucleic acid sequences can be inserted or deleted within a native plant sequence encoding the LOX (e.g., LOX-2, LOX-3) and/or FAD (e.g., FAD2B, FAD3C, FAD3D) protein through the use of methods available in the art. Such methods include, but are not limited to, meganucleases designed against the plant genomic sequence of interest (D′Halluin et al (2013) Plant Biotechnol J 11: 933-941); CRISPR-Cas9, CRISPR-Cpf1, TALENs, and other technologies for precise editing of genomes (Feng et al. (2013) Cell Research 23:1229-1232, Podevin et al. (2013) Trends Biotechnology 31: 375-383, Wei et al. (2013) J Gen Genomics 40: 281-289, Zhang et al (2013) WO 2013/026740, Zetsche et al. (2015) Cell 163:759-771, U.S. Provisional Patent Application 62/295,325); N. gregoryi Argonaute-mediated DNA insertion (Gao et al. (2016) Nat Biotechnol doi:10.1038/nbt.3547); Cre-lox site-specific recombination (Dale et al. (1995) Plant J 7:649-659; Lyznik, et al. (2007) Transgenic Plant J 1:1-9; FLP-FRT recombination (Li et al. (2009) Plant Physiol 151:1087-1095); Bxb 1-mediated integration (Yau et al. (2011) Plant J 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol 701: 51-65; Puchta (2002) Plant Mol Biol 48:173-182). As used herein, a Cpf1 nuclease refers to a Cas12a nuclease and the terms can be used interchangeably. The insertion of said nucleic acid sequences will be used to achieve the desired result of decreased expression of a gene encoding the LOX (e.g., LOX-2, LOX-3) and/or FAD (e.g., FAD2B, FAD3C, FAD3D) protein.

Alteration of gene encoding a LOX (e.g., LOX-2, LOX-3) and/or FAD (e.g., FAD2B, FAD3C, FAD3D) protein expression may also be achieved through the modification of DNA in a way that does not alter the sequence of the DNA. Such changes could include modifying the chromatin content or structure of the gene encoding a LOX (e.g., LOX-2, LOX-3) and/or FAD (e.g., FAD2B, FAD3C, FAD3D) protein of interest and/or of the DNA surrounding the gene encoding a LOX (e.g., LOX-2, LOX-3) and/or FAD (e.g., FAD2B, FAD3C, FAD3D) protein. It is well known that such changes in chromatin content or structure can affect gene transcription (Hirschhorn et al. (1992) Genes and Dev 6:2288-2298; Narlikar et al. (2002) Cell 108: 475-487). Such changes could also include altering the methylation status of the gene encoding one or more of LOX-2, LOX-3, FAD2B, FAD3C and FAD3D protein of interest and/or of the DNA surrounding the gene encoding one or more of LOX-2, LOX-3, FAD2B, FAD3C and FAD3Dprotein of interest. It is well known that such changes in DNA methylation can alter transcription (Hsieh (1994) Mol Cell Biol 14: 5487-5494). Targeted epigenome editing has been shown to affect the transcription of a gene in a predictable manner (Hilton et al. (2015) 33: 510-517). It will be obvious to those skilled in the art that other similar alterations (collectively termed “epigenetic alterations”) to the DNA that regulates transcription of the gene encoding a LOX (e.g., LOX-2, LOX-3) and/or FAD (e.g., FAD2B, FAD3C, FAD3D) protein of interest may be applied in order to achieve the desired result of an altered gene encoding a LOX (e.g., LOX-2, LOX-3) and/or FAD (e.g., FAD2B, FAD3C, FAD3D) protein.

Alteration of gene encoding a LOX (e.g., LOX-2, LOX-3) and/or FAD (e.g., FAD2B, FAD3C, FAD3D) protein expression may also be achieved through the use of transposable element technologies to alter gene expression. It is well understood that transposable elements can alter the expression of nearby DNA (McGinnis et al. (1983) Cell 34:75-84). Alteration of the expression of a gene encoding a LOX (e.g., LOX-2, LOX-3) and/or FAD (e.g., FAD2B, FAD3C, FAD3D) protein may be achieved by inserting a transposable element upstream of the gene encoding a LOX (e.g., LOX-2, LOX-3) and/or FAD (e.g., FAD2B, FAD3C, FAD3D) protein of interest, causing the expression of said gene to be altered.

Alteration of gene encoding a LOX (e.g., LOX-2, LOX-3) and/or FAD (e.g., FAD2B, FAD3C, FAD3D) protein expression may also be achieved through expression of a transcription factor or transcription factors that regulate the expression of the gene encoding LOX (e.g., LOX-2, LOX-3) and/or FAD (e.g., FAD2B, FAD3C, FAD3D). It is well understood that alteration of transcription factor expression can in turn alter the expression of the target gene(s) of said transcription factor (Hiratsu et al. (2003) Plant J 34:733-739). Alteration of gene encoding a LOX (e.g., LOX-2, LOX-3) and/or FAD (e.g., FAD2B, FAD3C, FAD3D) protein expression may be achieved by altering the expression of transcription factor(s) that are known to interact with a gene encoding a LOX (e.g., LOX-2, LOX-3) and/or FAD (e.g., FAD2B, FAD3C, FAD3D) protein of interest.

Alteration of gene encoding a LOX (e.g., LOX-2, LOX-3) and/or FAD (e.g., FAD2B,

FAD3C, FAD3D) protein expression may also be achieved through the insertion of a promoter upstream of the open reading frame encoding a native LOX (e.g., LOX-2, LOX-3) and/or FAD (e.g., FAD2B, FAD3C, FAD3D) protein in the plant species of interest. This may occur through the insertion of a promoter of interest upstream of a LOX (e.g., LOX-2, LOX-3) and/or FAD (e.g., FAD2B, FAD3C, FAD3D) protein-encoding open reading frame using a meganuclease or other suitable nuclease system designed to target the genomic sequence of interest. This strategy is well-understood and has been demonstrated previously to insert a transgene at a predefined location in the cotton genome (D′Halluin et al. (2013) Plant Biotechnol J 11: 933-941). It will be obvious to those skilled in the art that other technologies can be used to achieve a similar result of insertion of genetic elements at a predefined genomic locus by causing a double-strand break at said predefined genomic locus and providing an appropriate DNA template for insertion (e.g., CRISPR-Cas9, CRISPR-Cpf1, CRISPR-Cms1, TALENs, ZFNs, and other technologies for precise editing of genomes).

### 2.8. Breeding of Plants

Also disclosed herein are methods for breeding a plant, such as a pea (i.e., Pisum sativum) plant that contains mutated LOX (e.g., LOX-2, LOX-3) and/or FAD (e.g., FAD2B, FAD3C, FAD3D) gene, as described hereinabove. For example, disclosed herein are methods for breeding a plant that contains one or more of the mutated LOX-2, LOX-3, FAD2B, FAD3C and FAD3D genes and a corresponding decrease in LOX-2, LOX-3, FAD2B, FAD3C and/or FAD3D function. A plant containing a mutated LOX (e.g., LOX-2, LOX-3) and/or FAD (e.g., FAD2B, FAD3C, FAD3D) gene may be regenerated from a plant cell or plant part that contains mutated LOX (e.g., LOX-2, LOX-3) and/or FAD (e.g., FAD2B, FAD3C, FAD3D) gene. Using conventional breeding techniques or self-pollination, one or more seeds may be produced from the plant that contains mutated LOX (e.g., LOX-2, LOX-3) and/or FAD (e.g., FAD2B, FAD3C, FAD3D) gene, as described hereinabove. Such a seed, and the resulting progeny plant grown from such a seed, may contain mutated LOX (e.g., LOX-2, LOX-3) and/or FAD (e.g., FAD2B, FAD3C, FAD3D) gene and may express LOX (e.g., LOX-2, LOX-3) and/or FAD (e.g., FAD2B, FAD3C, FAD3D) protein with reduced function of loss-of-function. In certain instances, such a seed, and the resulting progeny plant grown from such a seed, may contain a polynucleotide of the present disclosure, and therefore may be transgenic. Progeny plants are plants having mutation in LOX (e.g., LOX-2, LOX-3) and/or FAD (e.g., FAD2B, FAD3C, FAD3D) gene that descended from the original plant with mutated LOX (e.g., LOX-2, LOX-3) and/or FAD (e.g., FAD2B, FAD3C, FAD3D) gene. Seeds produced using such a plant of the invention can be harvested and used to grow generations of plants having mutated LOX (e.g., LOX-2, LOX-3) and/or FAD (e.g., FAD2B, FAD3C, FAD3D) gene. Additionally, such progeny plants may express a gene of agronomic interest (e.g., herbicide resistance gene). Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting. Unless otherwise noted, all parts and percentages are by dry weight.

### EXAMPLES

### Example 1. Materials and Methods

### Transformation

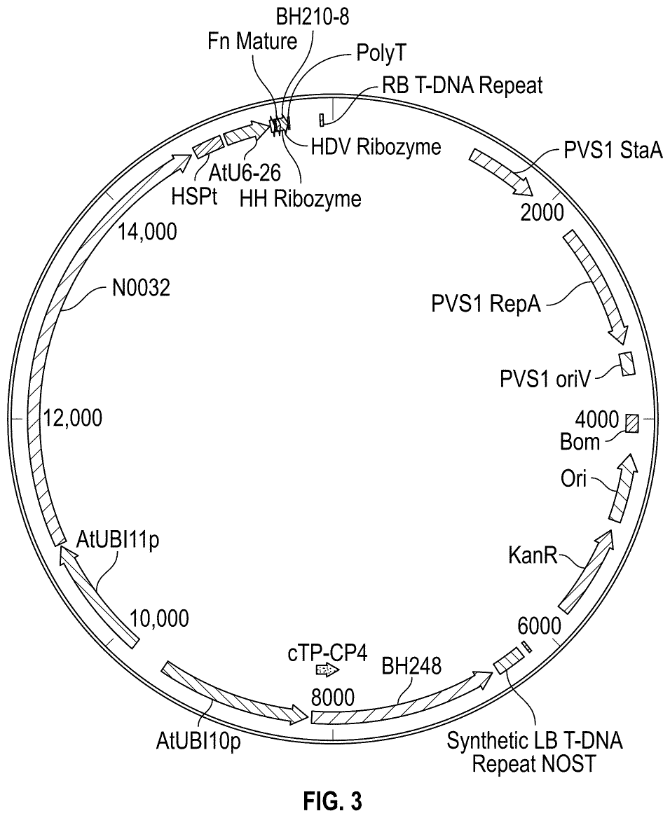

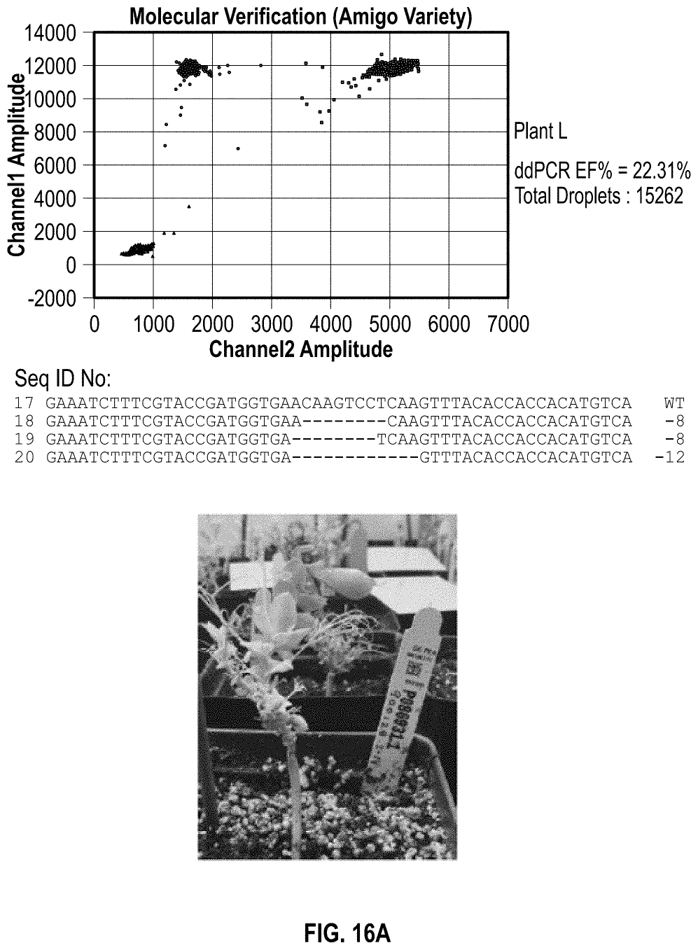

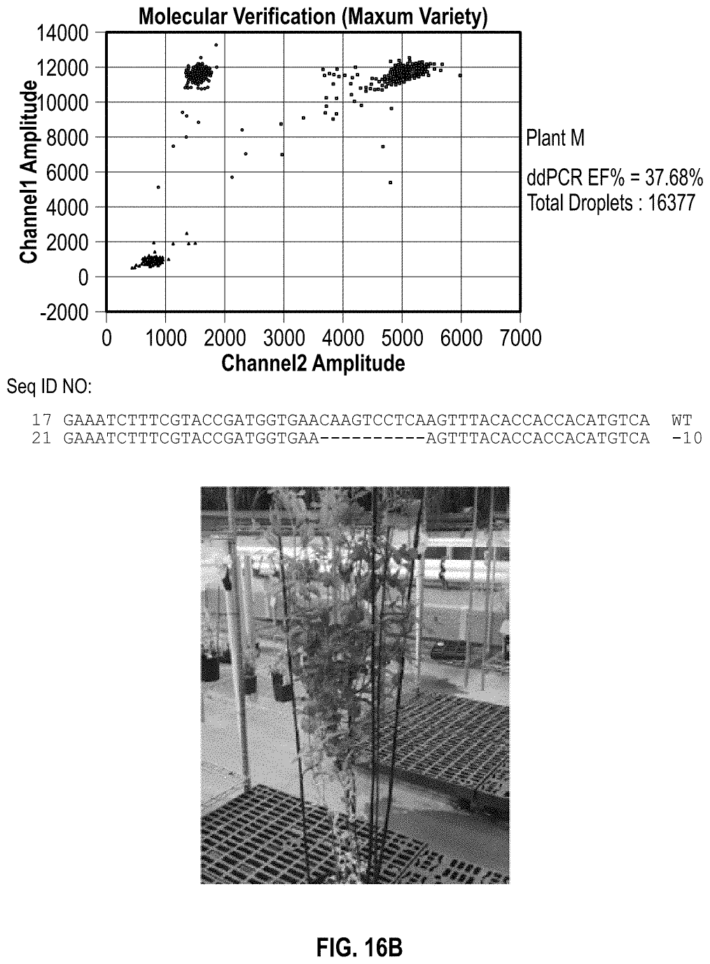

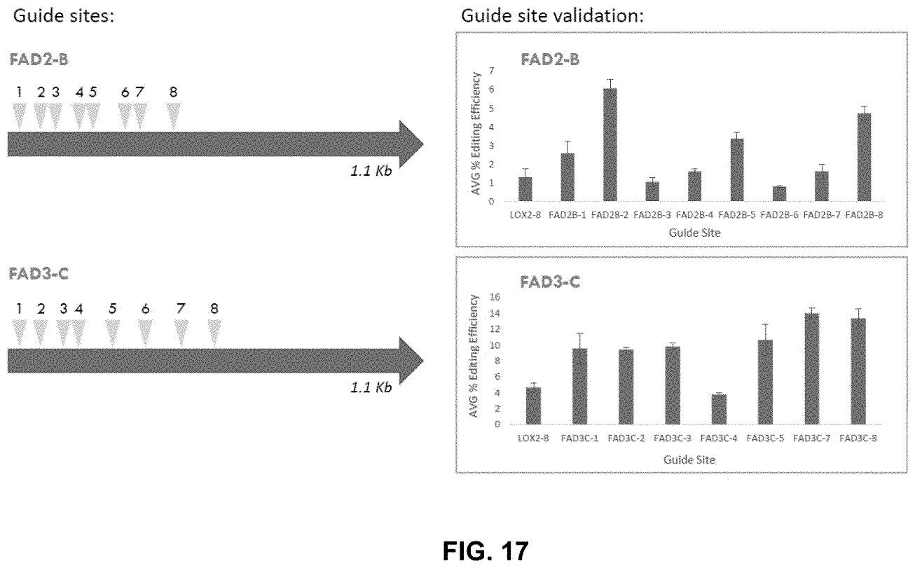

Embryonic axes of mature seeds of yellow pea varieties Amigo and Maxum were transformed with construct A (FIG. 3) using Agrobacterium transformation. Embryonic axes were infected using Agrobacterium tumefaciens strain AGL1 carrying construct A (CP4 selectable marker cassette conferring resistance to glyphosate; nuclease (e.g., a RNA guided nuclease) and gRNA cassettes to drive genome editing at the LOX-2 gene locus). After co-cultivation for 3 days, embryonic axes were grown in selective media to promote shoot formation of transformed explants. Tissue was sub-cultured every two weeks until 2-3 inch transgenic shoots were robust, green and had expanding leaves and tendrils. At which point they were micrografted onto wild-type (WT) Amigo pea etiolated rootstocks and cultured 1-3 weeks at 21-25° C. under 100 μmol m⁻¹ s⁻² until graft union was established, and then transferred to soil. Transgenic events were recorded, the T0 plants were assigned unique Plant Names (e.g., Plant A, Plant B) and subjected to molecular characterization and propagation. Transformed T0 plants were identified, amplicons produced of the genomic regions near the targeted LOX 2 gene, and sequenced. Plant E contained an 11 bp deletion in exon 4 of the LOX 2 gene (SEQ ID NO: 5) and was selected for further development. The Maxum variety of LOX-2 edited pea plant was obtained using the method described above. Briefly, embryonic axes of mature seeds of yellow pea variety Maxum was transformed with LOX-2 variant constructs shown in FIG. 3 using Agrobacterium transformation and an LOX2 edited yellow pea plant was successfully generated (FIG. 16B). Transgenic events were recorded, the T0 plant was assigned unique Plant Name Plant M and subjected to molecular characterization and propagation. Transformed T0 plants were identified, amplicons produced of the genomic regions near the targeted LOX 2 gene, and sequenced. Pea lines used in the study are described in Table 1 below.

TABLE 1
Pea Lines Used in the Study

| ID | Description | Use in Study |
|---|---|---|
| WT | WT Amigo Variety | Negative Control for NGS |
| Plant K | Transformed T0 Plant | Amplicon DNA Sequencing |
| Plant C | T1 Edit null/T-DNA Null | Negative Control for NGS |
| Plant D | T1 HOM edit/HEMI T-DNA | Positive Control for NGS |
| | single copy | |
| Plant E | T1 HOM edit/T-DNA null | Test Sample for NGS |
| WT-1 | WT-1 Amigo Variety | Negative Control for NGS |
| Plant L | Transformed T0 Plant | Amplicon DNA Sequencing |
| WT-2 | WT-1 Maxum Variety | Negative Control for NGS |
| Plant M | Transformed T0 Plant | Amplicon DNA Sequencing |

### Production & Purification of the Reference Proteins

One liter of cell pellet was resuspended in 35 mL lysis buffer (50 mM NaH2PO4 pH 7.8, 300 mM NaCl, 1 mg/ml lysozyme, 2 tablets of proteinase inhibitor, 2 mM MgCl2) and resuspended samples were stored at −80° C. for future purification. Samples were then flash-frozen using liquid nitrogen and allowed to thaw. Additional lysis buffer (approximately 15-20 mL) was added to resuspend the samples and incubated on ice for 30 minutes. The resuspended cell pellet was sonicated with 5 seconds sonication, 10 seconds break, for 1 minute/12 cycles at 40% power. Then, 5 μL of nuclease was added to the cell lysate and incubated for 30 min on ice. The lysate was transferred to Nalgene High-Speed Centrifuge tubes and debris was cleared by centrifugation in an Eppendorf 5804R, F-34-6-38 rotor at 11,000×rpm for 25 minutes at 4° C.

The cleared lysate was incubated with Protino resin (0.2 g of resin/1 L cell culture) at 4° C. for 1 h with low speed rotation. The lysate and resin were loaded into disposable column at 4° C. The column was washed with 25 mL of wash buffer (50 mM NaH2PO, pH 7.8, 300 mM NaCl). The column was then eluted with 8 ml of elution buffer (50 mM NaH2PO4, pH 7.8, 300 mM NaCl, 50 mM imidazole). The eluted protein was concentrated by a centrifugal concentrator in an Eppendorf 5804R, S-4-72 rotor centrifuge at 4,000×rpm. The concentrated protein was dialyzed with dialysis buffer (50 mM NaH2PO4, pH 6.3) for 20 h at 4° C. with shaking. Identity of the purified LOX-2 protein was determined by western blot analysis using a protein specific antibody (Lox2-L3 antibody). Identities of the purified LOX-3, FAD2B, FAD3C and FAD3D proteins were determined by western blot analysis using a corresponding protein specific antibody.

### PAGE Gel and Western Blot Analysis

5 μg of total protein extracted from yellow pea seeds and 500 ng of purified His-LOX-2 were loaded on 8% NuPAGE gel (Invitrogen) and run at 200 V for 2 h. Proteins from the gel were transferred to PVDF membrane according to manufacturer's instruction (Bio-Rad). After transferring, the membrane was incubated in blocking buffer (5% milk in 0.05 PBST) for 1 h at room temperature. Subsequently, primary antibody (Lox2-L3 antibody) with 1:1000 dilution was added to blocking buffer and incubated for 1 h at room temperature. Membrane was washed five times with 0.05% PB ST buffer, then secondary antibody (anti-rabbit) was added to 0.05% PB ST and incubated 1 h at room temperature. After washing membrane five times with 0.05% PBST buffer, membrane was developed to detect signal using Bio-Rad ChemiDoc system. A similar PAGE gel and western blot analysis protocol to the one described above, was performed with corresponding primary antibodies (e.g., Lox2-L3 antibody, FAD2 antibody, FAD3 antibody) to detect/identify purified LOX-3, FAD2B, FAD3C and FAD3D proteins.

Total Protein Preparation from Yellow Pea Seed

Dry seeds were ground to a fine powder and defatted with hexane (1:4 ratio, 1 g of powder/4 ml hexane) by rotating sample overnight. Hexane was removed by evaporation to dryness using vacuum pump. Total crude proteins were extracted from defatted powder in 10 volumes of 50 mM sodium phosphate buffer (pH 6.3) by rotating 5 h at 4° C. The extract was cleared by centrifugation at 12,000 rpm for 20 min and supernatant was used as total crude protein for LOX-2 enzyme assay and western blot for LOX-2 protein detection.

### LOX-2 Enzyme Assay

Lipoxygenase converts the LOX substrate to an intermediate that reacts with the probe generating a fluorescent product. The main products of fatty acid oxidation for LOX-2 are 13-HPODE and 9-HPODE with 7: 1 ratio, the former is known to be responsible for hexanal production. The increase in fluorescent signal can be recorded at 500/546 nm (Ex/Em) in kinetic mode for 30 min and is directly proportional to LOX activity. For this study, linolenic acid was used as a substrate for LOX-2 enzyme assay. Linolenic acid (2.8 μL) was dispersed by gentle vortex in 1 mL water containing 0.28% (v/v) Tween 20, 0.011 M NaOH to give 10 mM stock substrate. Each reaction contains 1 μg total protein, 2 μL stock substrate, 2 μL probe (1:10 in DMSO), 50 mM sodium phosphate buffer (pH 6.3) in total 200 μL reaction. Reagents were added in order from buffer, protein, substrate to probe and immediately start recoding fluorescence at 30 second intervals for 30 min. Reaction without substrate used as a background control. Data analysis has been performed by calculating [symbol] RFU at linear change in signal between time t1 and t2 ( [symbol] t).

### Analysis of Volatiles by Solid-Phase Micro-Extraction (SPME) and Gas Chromatography

One yellow pea seed was cracked, dehulled and ground in a TissueLyser II (QIAGEN). Pea flour (200 mg) was placed in a glass vial with 2 ml water and incubated for 2 h at room temperature with shaking. Sodium chloride (0.2 g/mL sample) was added into each vial to reduce matrix effects and improve the transfer of volatile compounds to the fiber. Finally, 30 μL of internal standard (methyl decanoate, 0.01 ppt in methanol) was added into each 2 mL sample. The SPME fiber (DVB/CAR/PDMS, Sigma) was inserted into the gas chromatograph injection port for 5 min at 250° C. to clean the fiber before each extraction. The sealed samples were held with stirring at 45° C. for 15 min to equilibrate the sample and its headspace; the SPME fiber was inserted into the vial for 30 min at 45° C. to extract volatile compounds. When the extraction was complete, the fiber was immediately transferred to the injection port of the GC equipped with a DB-WAX column (30 m×0.25 mm, 0.25 μm, Agilent), to desorb the volatile compound at 250° C. for 2 min. The carrier gas (helium) flow rate was 1.2 ml/min. The initial oven temperature was held at 35° C. for 1 min and then gradually increased to 220° C. at a rate of 10° C./min and held for 5 minutes at each temperature. The injector and detector temperature were set at 250° C. The injection port was set to a splitless mode. Peaks were identified by comparing their retention times. Standards such as n-Hexanal, n-hexan-1-ol, 1-octen-3-ol, 2-octanol and n-pentan-1-ol were obtained from Sigma and were of analytical grade.

### Editing Efficiency Calculation

Amplicon sequencing results are reported as editing efficiency. Editing efficiency is calculated based on the percentage of edited reads to total aligned reads and an edited read was recorded for any sequence with >2 reads containing a deletion at the predicted cleavage site.

### Guide RNA Design

A guide RNA used to produce the Pea LOX-2 with the 11 bp deletion was designed according to standard methods of the art (Zetsche et al., Cell, Volume 163, Issue 3, Pages 759-771, 2015; Cui et al., Interdisciplinary Sciences: Computational Life Sciences, volume 10, pages 455-465, 2018). Similarly, guide RNAs used to produce the Pea LOX-3, FAD2B, FAD3C, FAD3D each with a 24 bp deletion were designed according to standard methods of the art. Optimized gRNA design to maximize activity and minimize off-target effects of CRISPR-Cas9 and CRISPR-Cas12a have been extensively characterized (Nat Biotechnol 34, 184-191, doi: 10.1038/nbt.3437 (2016)). The CRISPR-Cas12a system described herein can be employed for targeting PAM sites such as TTN, TTV, TTTV, NTTV, TATV, TATG, TATA, YTTN, GTTA, and GTTC, utilizing corresponding gRNAs.

### DNA Sequencing and Analysis

Illumina sequencing was performed on one test sample and three control samples, as described in Table 2.

TABLE 2
DNA Samples Utilized in the Study

| Sample Type | Sample ID | Description |
|---|---|---|
| Negative Control | WT | WT Amigo |
| Negative Control | Plant C | Edit null/T-DNA null |
| Positive Control | Plant D | HOM edit/HEMI T-DNA single copy |
| Test | Plant E | HOM edit/T-DNA null |

Four samples tested in this study included two negative controls (WT & Plant C), one positive control (Plant D), and one test sample (Plant E). The Pisum sativum genome, as described, for example, in Kreplak et al. (Nature Genetics, 51:1411-1422 (2019)), was used as the reference genome for all subsequent analyses. All Illumina sequences were subjected to sequence quality control to confirm their suitability for further analysis. The FastQC (v0.11.7) program was used to perform this quality control. The results of QC analysis of the Illumina sequencing data are shown in Table 3.

TABLE 3
Illumina Sequencing QC Results

| | | | Nucleotide | |
|---|---|---|---|---|
| | Sequence | Sequence | Count | FastQC |
| Sample | Count | Length | (bases) | Pass/Fail |
| WT | 1,188,990,720 | 151 | 179,537,598,720 | Pass |
| Plant C | 1,293,293,798 | 151 | 195,287,363,498 | Pass |
| Plant D | 1,435,861,128 | 151 | 216,815,030,328 | Pass |
| Plant E | 1,228,170,740 | 151 | 185,453,781,740 | Pass |

All sample data passed QC and were suitable for further analyses. The sequence data was further analyzed to determine whether enough data was generated to ensure a complete and comprehensive study. This analysis was achieved by assessing the per-sample coverage of all known single copy loci contained within the pea genome. All conserved single copy gene loci belonging to the order Fabales were gathered from the OrthoDB database (v10.1) and mapped to the pea reference genome. The effective coverage distribution was assessed, and outliers were trimmed if necessary. The final set of 4,570 loci covered all chromosomes of the pea reference genome. Mapping of the Next Generation Sequencing (NGS) data to these single copy loci was used as an internal standard to assess coverage and completeness (Table 4) and to normalize study data for the per-sample variable sequencing depth.

TABLE 4
Estimated x-fold coverage and completeness
of coverage in samples

| | Mean Diploid | |
|---|---|---|
| | x-fold Coverage | Completeness |
| Sample | (Standard Deviation) | of Coverage |
| WT | 43.33 (6.0) | 100.00000% |
| Plant C | 42.80 (6.3) | 100.00000% |
| Plant D | 48.54 (6.9) | 100.00000% |
| Plant E | 41.22 (6.1) | 100.00000% |

Effective Coverage was determined by mapping the appropriate Illumina NGS data to the single copy loci sequences and calculating mean depth of coverage across their length, reported as coverage expected on a diploid basis. Coverage completeness is an estimation of the total percentage of all bases in the pea genome which would be expected to be covered by NGS data given the samples' x-fold coverage shown:
Completeness=1−e^{-x fold coverage} (Clarke and Carbon, Cell 9.1: 91-99 (1976))

These data show that all samples were comprehensively covered, and further study of these data would allow an accurate determination of the presence or absence of transformation plasmid sequences in the test events.

### Example 2. Pea LOX-2 Gene

### Pea LOX-2 Gene Structure

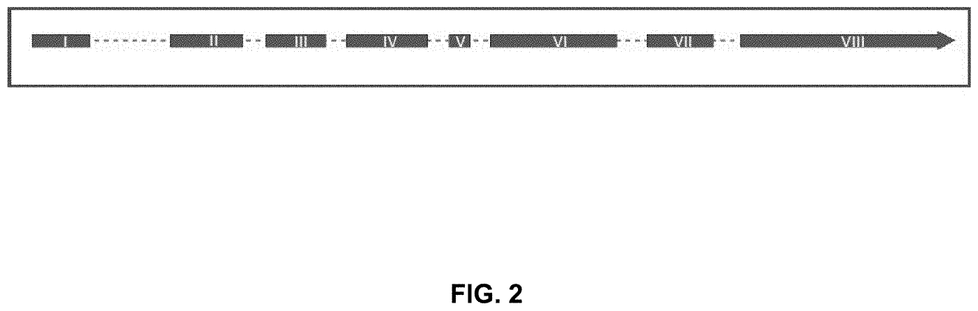

The pea LOX-2 gene was determined by DNA sequence analysis of genomic DNA isolated from pea variety Amigo and is shown in FIG. 2. As described in FIG. 2, the LOX-2 gene consists of 8 exons and 7 introns.

### Guide RNA Design for Pea LOX-2 Gene Mutation

A guide RNA specific to a region (SEQ ID NO: 15) in exon 4 of the Pea LOX-2 gene (SEQ ID NO: 3) was synthesized according to standard procedures.

Pea embryonic axis were transformed with construct A (FIG. 3) containing a gRNA that has a 24 nucleotide targeting region (encoded by SEQ ID NO: 4) specific to exon 4 of the pea LOX 2 gene (SEQ ID NO: 3). Transformed plants were identified by their resistance to glyhphosate and amplicons were produced of the genomic regions near the targeted LOX-2 gene and sequenced. Amplicons with deletion in exon 4 of LOX-2 gene were identified by using forward primer 13062

(SEQ ID NO: 13) and reverse primer 13057 (SEQ ID NO: 14). Plant E contained an 11 bp deletion in exon 4 of the LOX-2 gene (SEQ ID NO: 5) and was selected for further development. Plant E was self-pollinated and the T1 generation was used for determining the presence/absence of gene editing machinery.

Amigo mature seed embryonic axis were transformed with construct 134164 using Agrobacterium transformation. Embryonic axes were infected using Agrobacterium tumefaciens strain AGL1 carrying construct 134164. Transformed T0 plants were identified, and amplicons were produced of the area around the targeted LOX 2 gene and sequenced. Plant B contained an 8 bp deletion in exon 4 of the LOX 2 gene (SEQ ID NO: 6) and was selected for further development. Plant B was self-pollinated and the T1 generation was used for determining the presence/absence of gene editing machinery.

### Identification of Potential Off-Target Sites

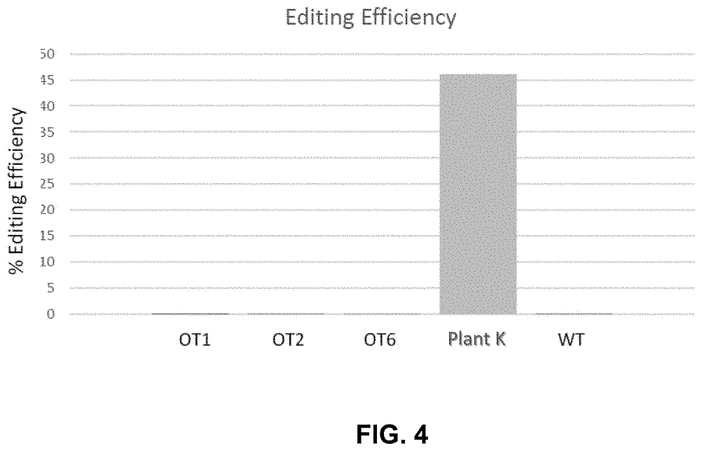

The gRNA sequence was used to query the Amigo genomic DNA sequence to determine any potential off-target sites. Three potential off-target sites with 3 or less mismatches were identified in the amigo genome. The off-target sequences are provided. Each of these regions were amplified and sequenced. The editing efficiency at each site was compared to the editing efficiency at the targeted site in T0 Plant K and the WT plant. The results are described in FIG. 4. As shown in FIG. 4, the DNA locations identified as potential off-target sites for the LOX-2 guide RNA were not edited.

### Pea LOX-2 Gene Summary

The Pea LOX-2 gene was knocked out by an 11 bp deletion in exon 4 in Plant E. Plant E was further characterized by whole genome sequencing and bioinformatic analysis. The NGS data analyzed was of good quality and in sufficient quantity to assure a comprehensive study for all samples. Two separate methods showed that transformation plasmid sequences were not present in the test sample nor were they integrated into the genome of the test sample. In addition, three potential off-target sites were identified and analyzed by DNA sequencing of amplicons representing these regions. There was no evidence that any gene editing occurred at these off-target sites.

Alternatively, the Pea LOX-2 gene was knocked out by an 8 bp deletion in exon 4 in Plant B. Plant B was further characterized by whole genome sequencing and bioinformatic analysis. The NGS data analyzed was of good quality and in sufficient quantity to assure a comprehensive study for all samples. Two separate methods showed that transformation plasmid sequences were not present in the test sample nor were they integrated into the genome of the test sample. In addition, three potential off-target sites were identified and analyzed by DNA sequencing of amplicons representing these regions. There was no evidence that any gene editing occurred at these off-target sites.

### Example 3. LOX-3, FAD2, and/or FAD3-Mutated Pea Plant

Guide RNA Design and Evaluation for Pea LOX-3, FAD2B, FAD3C and/or FAD3D Gene Knockout

A guide RNA (encoded by SEQ ID NO: 23) specific to a region exon 4 pf the Pea LOX-3 gene (SEQ ID NO: 22), a guide RNA (encoded by SEQ ID NO: 30) specific to a region exon 1 of the Pea FAD2B gene (SEQ ID NO: 29), a guide RNA (encoded by SEQ ID NO: 40) specific to a region exon 2 of the Pea FAD3C gene (SEQ ID NO: 39), a guide RNA (encoded by SEQ ID NO: 50) specific to a region exon 3 of the Pea FAD3D gene (SEQ ID NOs: 49), were synthesized according to standard procedures. Pea embryonic axis were transformed with constructs containing the corresponding gRNAs specific to each of the pea LOX-3, FAD2B, FAD3C or FAD3D genes. Table 5 shows the profiles of the plants obtained, comprising mutations in one or more of the LOX-3 gene, the FAD2B gene, the FAD3C gene, and the FAD3D gene. For obtaining pea plants with the edits of the LOX-3, FAD2, and/or FAD3 gene shown in Table 5, pea embryonic axis was transformed with constructs containing the corresponding gRNAs specific to each of the pea LOX-3, FAD2B, FAD3C, and/or FAD3D genes. Transformed Pisum sativum plants were identified by their resistance to glyhphosate and amplicons were produced of the genomic regions near the targeted LOX-3, FAD2, FAD3C, and/or FAD3D genes and sequenced. Amplicons with deletion in targeted LOX-3, FAD2, FAD3C, and/or FAD3D genes were identified by using forward and reverse primers with nucleotide sequences set forth in Table 9. The detection frequency of deletion in the target is set forth as “ddPCR%” in Table 5.

TABLE 5
Pea plants with mutations in the
LOX-3, FAD2, and/or FAD3 genes*

| Inoculation Reagent | Gene Target | ddPCR % |
|---|---|---|
| Reagent A | fad2b | 54.9 |
| Reagent B | fad2b | 38.2 |
| Reagent C | fad2b/fad2c | 40.8, 15.3 |
| Reagent D | fad2b/fad3c/fad3d | 51.5 |
| Reagent E | fad2b/fad3c/fad3d | 46.2, 94.4, 77.6 |
| Reagent F | lox-3 | 54.3 |

*Mutated alleles again confirmed at T1 stage and results remain consistent with original T0 next generation sequencing (NGS) results.

### Pea FAD2B Gene Summary

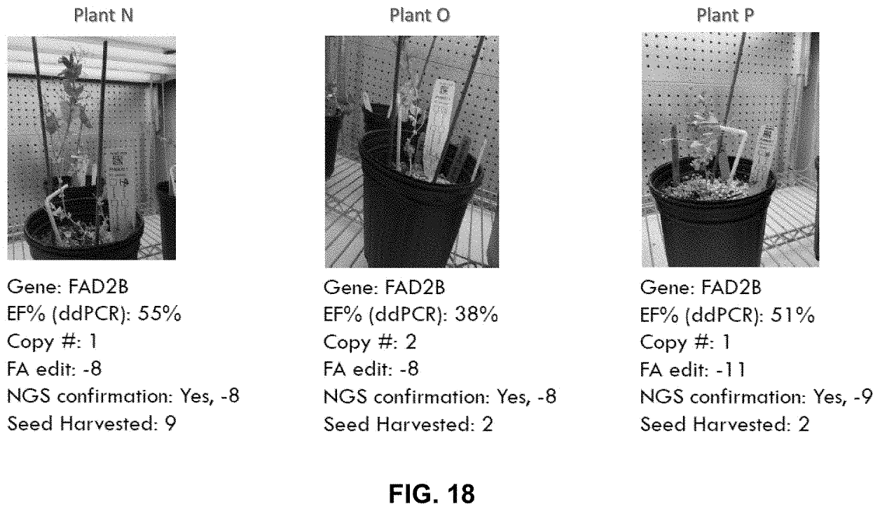

The Pea FAD2B gene was knocked out in Plants N, O, and P, respectively and the plants were further characterized by whole genome sequencing and bioinformatic analysis (FIG. 18). The NGS data analyzed was of good quality and in sufficient quantity to assure a comprehensive study for all samples. Two separate methods showed that transformation plasmid sequences were not present in the test sample nor were they integrated into the genome of the test sample. There was no evidence that any gene editing occurred at these off-target sites.

### Example 4. Improved Flavor Pea Plant Characterization

### Mutated Pea LOX-2 Gene Does Not Produce Detectable Levels of Full-Length LOX-2 Protein

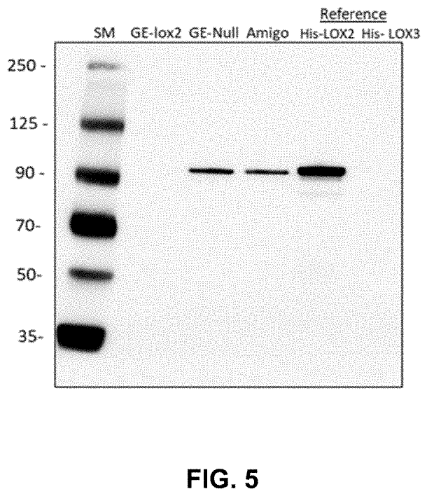

The edited LOX-2 gene is predicted to produce a truncated protein of 358 amino acids representing the amino terminus of the protein and lacking the enzyme's active site located in exon 7 (Wang et al., Proc Natl Acad Sci USA 91: 5828-5832 (1994)). A Lox2-L3 antibody was produced to amino acids 409-429 of the full-length LOX-2 protein. When the Lox2-L3 antibody was used in a western blot analysis of seed protein extracts from the edited LOX-2 gene pea plant, the full-length protein (Mw approx. 97.1 kDa) was not detectable. However, the antibody recognized the LOX-2 protein in non-edited seeds and the LOX-2 reference protein (FIG. 5).

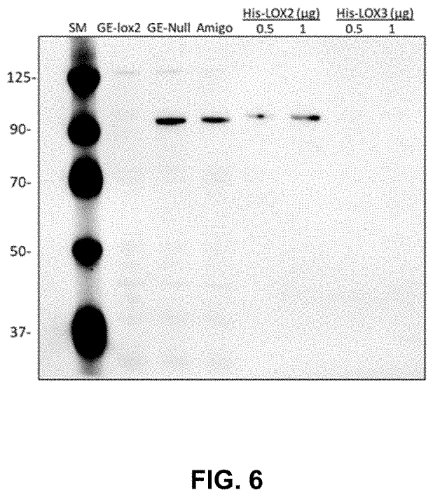

A Lox2-L1 antibody was produced to amino acids 11-25 of the full-length LOX-2 protein. When the Lox2-L1 antibody was used in a western blot analysis of seed protein extracts from the edited LOX-2 gene pea plant, the full-length protein was also undetectable. However, the antibody recognized the LOX-2 protein in non-edited seeds and the LOX-2 reference protein (FIG. 6).

In addition, the Lox2-L1 antibody did not recognize a truncated version (Mw approx. 39.7 kDa) of the LOX-2 protein in the edited LOX-2 gene pea plant (FIG. 6, Lane GE-1ox2). Without being limited by theory, this may result from the rapid degradation of the protein because of improper folding of the truncated LOX-2 protein (Preiss et al., Eur J Biochem 268: 4562-4569 (2001)).

### Improved Flavor Pea Plant Seeds Have Reduced LOX-2 Enzyme Activity

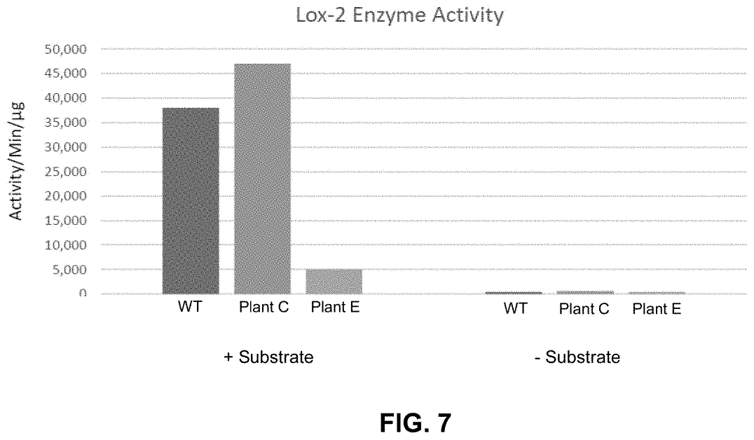

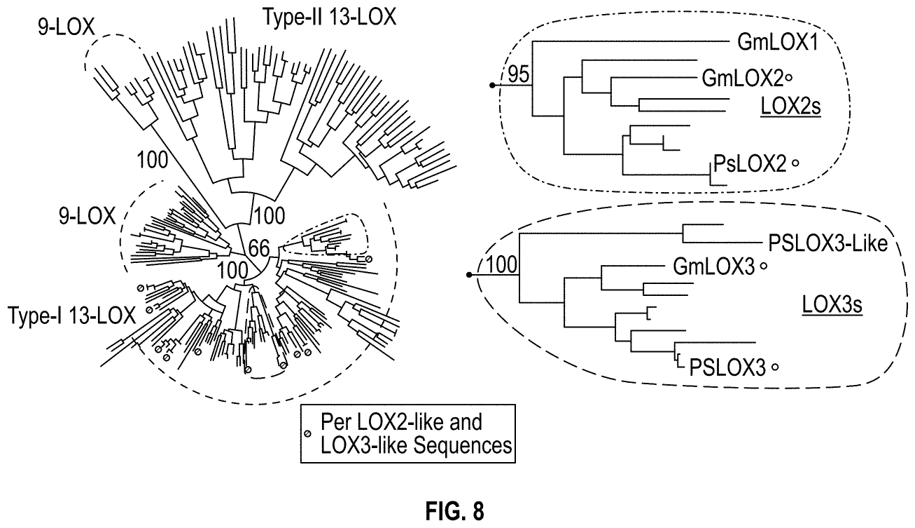

Total seed protein from Plant C , Plant E and WT were used to assess LOX-2 enzyme activity. LOX-2 activity was monitored by measuring increased fluorescence signal, which is generated by the reaction of probe with oxidized fatty acid when substrate and LOX-2 protein are present in the reaction. LOX-2 activity was significantly reduced in seed from the edited LOX-2 gene plant (Plant E) compared to that of non-edited plant (Plant C) and wild-type plant, respectively (FIG. 7).

### Improved Flavor Pea Plant Seeds Have Reduced Amounts of Hexanal and Hexanol

Oxylipin metabolism was analyzed in pea homogenates from seed flour of plants that had edited LOX-2 genes (Plant B Plant B (having a 8 bp deletion) and Plant E (having a 11 bp deletion) and compared to seed flour homogenates of non-edited plants (Plant A and Plant C; null segregants of the edited plants Plant B and Plant E, respectively). The values of a panel of volatile products were generated using gas chromatography mass spectrometry (GC-MS). SPME-GC analysis showed two C6 volatiles, n-hexanal and hexanol, to be major volatiles in non-edited plant seed flour. The relative peak area was used to compare the intensity of volatile production in the edited LOX-2 gene plant and the non-edited plant. In Table 6, the amount of each compound in the negative control plants (Plant A and Plant C) was set as 100% and compared to the amount of each compound in the Pea LOX-2 edited plants (Plant B and Plant E). In a separate experiment, in Table 7, the level of each compound in LOX-2 knockout plants (Plant B and Plant E), and their null segregants (Plant B and Plant E, respectively) were expressed by % change relative to that in a WT plant. As shown in Tables 6 and 7, levels of hexanal, 1-hexanol, and other volatiles were significantly reduced in samples from LOX2-knockout plants Plant B and Plant E as compared to their respective null segregant counterparts or a WT plant.

Yield and protein content were also assessed in these plants. Harvested seeds were scanned with a NIR analyzer and protein values were generated from our NIR prediction model. The results from the field trial (yield and total protein) demonstrated that LOX-2 knockout plants do not have a significant negative impact on the yield potential or total protein amount of the seed relative to a negative control (WT) plant.

TABLE 6
Intensity of Volatile Compounds in
LOX-2 Knockout and Control Plants

Relative Peak Area (%)

| Compound | Plant A | Plant B | Plant C | Plant E |
|---|---|---|---|---|
| Hexanal | 100 | 9.0 ± 0.85 | 100 | 12.6 ± 4.3 |
| 1-Hexanol | 100 | BDL | 100 | 13.9 ± 2.2 |
| l-octen-3-ol | 100 | BDL | 100 | BDL |

BDL = below detection limit

TABLE 7
Percent Change (Relative to WT) in the Volatile
Compounds in LOX-2 Knockout and Control Plants

| | Plant C | Plant E | Plant A | Plant B |
|---|---|---|---|---|
| Hexanal | 78.1 ± 15.5 * | −77.9 ± 1.6 ** | −33.9 ± 7.8 | −75.5 ± 1.7 ** |
| 1-Hexanol | 60.3 ± 9.9 * | −82.3 ± 0.3 ** | −32.3 ± 3.1 | −81.3 ± 1 ** |
| Pentanal | 91.5 ± 5 ** | 0.0 ± 0.0 | −26.1 ± 4.3 * | 0.0 ± 0.0 |
| 1-Pentanol | 34.9 ± 3.4 * | −50.9 ± 3.4 ** | −22.3 ± 3.3 * | −43.9 ± 2.2 ** |
| 1-Penten-3-ol | 16.3 ± 4.5 * | −63.1 ± 2.8 ** | −26.7 ± 2.7 * | −59.8 ± 2.6 ** |
| Heptanal | 80.1 ± 6.6 ** | −29.7 ± 7.3 * | −22.2 ± 6.5 | −26.3 ± 4.2 * |
| 1-Heptanol | 61.9 ± 8.7 * | −37.4 ± 3.1 * | −13.6 ± 2.0 | −29.2 ± 5.4 * |
| Octanal | 137.5 ± 20.5 * | −34.4 ± 6.2 * | −1.6 ± 9.5 | −37.5 ± 3.8 ** |
| 2-Octenal, (E) | 70.7 ± 5.5 ** | −39.8 ± 5.1 * | −12.7 ± 5.3 | −31.6 ± 4.1 * |
| 1-Octen-3-ol | 103 ± 0.9 ** | −8.1 ± 0.9 | −1.1 ± 0.2 | 0.5 ± 3.4 |
| 1-Octanol | 99.8 ± 11.1 * | 109.3 ± 3.8 ** | −25.1 ± 1.1 | 34.2 ± 6.7 * |
| Furan, 2-pentyl | −7.9 ± 1.8 | 64.9 ± 2.8 ** | −5 ± 3.3 | 56.5 ± 3 ** |
| Nonanal | 102.2 ± 9.6 ** | 15.3 ± 2.2 | −8.4 ± 2.5 | −8.5 ± 3.6 |

Significant difference relative to WT:
* p-value < 0.05;
*** p-value < 0.001;
*** p-value < 0.00

Improved Flavor Pea Plant Homogenates with Edited LOX-3 and FAD3C have Reduced Amounts of Hexanal and Hexanol

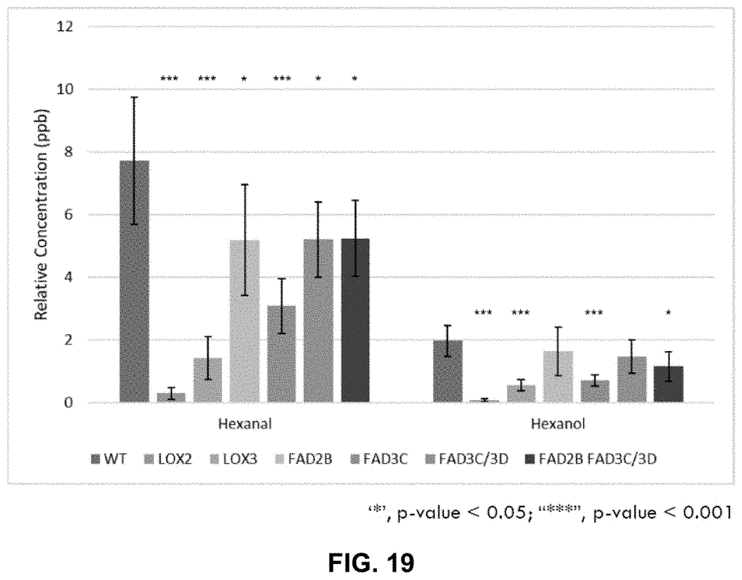

Pea analysis for off flavor compounds was performed in pea plants whose lipoxygenases and desaturases were edited. Pea homogenates from seed flour of plants that had edited LOX-3 and FAD3C genes was compared to homogenates of non-edited plants. SPME-GC analysis showed two C6 volatiles, n-hexanal and hexanol, to be major volatiles in non-edited plants. The relative peak area was used to compare the intensity of volatile production in the edited with edited LOX-3 and FAD3C gene plant and the non-edited plant. There was also a significant reduction of off-flavor compounds such as hexanal and hexanol in LOX-3 and FAD3C mutation yellow pea lines as shown in FIG. 19.

### Improved Flavor Pea Plant Seed Characterization Summary

Pea plants with an edited LOX-2 gene did not produce any detectable full-length protein as expected. Surprisingly and unexpectedly, even the truncated LOX-2 protein was not detected in the edited pea plants. In addition, the LOX-2 enzyme activity was greatly reduced in the edited plant and there was a concomitant reduction in hexanal and hexanol.

### Changes in Flavor Compounds in Knockout Lines Compared to Wild Type

Pea analysis for off flavor compounds was performed in knock out lines of pea plants whose lipoxygenases or desaturases were knocked out. Pea homogenates from seed flour of plants that had a knockout of LOX-2, LOX-3, FAD2B, FAD3C, a combination of FAD3C and FAD3D, or a combination of FAD2B, FAD3C and FAD3D genes, were compared to homogenates of wild-type plants.

Plant E contained a mutated LOX-2 gene comprising a sequence of SEQ ID NO: 11, with an 11 bp deletion in exon 4 of the LOX-2 gene.

Plant F contained a mutated LOX-3 gene comprising a sequence of SEQ ID NO: 28, with a 28 bp deletion in exon 4 of the LOX-3 gene.

Plant G contained a mutated FAD2B gene comprising a sequence of SEQ ID NO: 37 with an 8 bp deletion in exon 1 of the FAD2B gene.

Plant H contained a mutated FAD3C gene comprising a sequence of SEQ ID NO: 47 with an 8 bp deletion in exon 2 of the FAD3C gene.

Plant I contained a mutated FAD 3C gene comprising (i) a sequence of SEQ ID NO: 47 with an 8 bp deletion in exon 2 and (ii) a sequence of SEQ ID NO: 48 with a 49 bp deletion partially in exon 2 of the FAD3C gene, and a mutated FAD3D gene comprising (i) a sequence of SEQ ID NO: 57 with a 5 bp deletion in exon 3 and (ii) a sequence of SEQ ID NO: 58 with a 107 bp deletion partially in exon 3 of the FAD3D gene.

Plant J contained a mutated FAD 2B gene comprising a sequence of SEQ ID NO: 38 with a 2 bp deletion in exon 1 of the FAD2B gene, a mutated FAD3C gene comprising (i) a sequence of SEQ ID NO: 47 with an 8 bp deletion in exon 2 and (ii) a sequence of SEQ ID NO: 48 with a 49 bp deletion partially in exon 2 of the FAD3C gene, and a mutated FAD3D gene comprising a sequence of SEQ ID NO: 58 with a 107 bp deletion partially in exon 3 of the FAD3D gene.

SPME-GC analysis showed two C6 volatiles, n-hexanal and hexanol, to be major volatiles in wild-type plants. The relative peak area was used to compare the intensity of volatile production in the knockout lines of LOX-2, LOX-3, FAD2B, FAD3C, FAD3C and FAD3D, or FAD2B and FAD3C and FAD3D and the wildtype plant. There was a significant reduction of off-flavor compounds such as, hexanal and hexanol in the knockout lines compared to the wild type as shown in Table 8 below.

TABLE 8
Reduction in the Volatile Compounds in
Knockout lines Compared to Wildtype

| | | % Decrease in | % Decrease in |
|---|---|---|---|
| Plant ID | Genotype | Hexanal | 1-Hexanol |
| Plant E | lox-2 | 96% ± 2.4%*** | 96% ± 2.9%*** |
| Plant F | lox-3 | 82% ± 8.9%*** | 72% ± 9.2%*** |
| Plant G | fad2b | 33% ± 23.0%* | 17% ± 39.1% |
| Plant H | fad3c | 60% ± 11.3%*** | 64% ± 8.7%*** |
| Plant I | fad3c/fad3d | 33% ± 15.4%* | 25% ± 26.5% |
| Plant J | fad2b/fad3c/fad3d | 32% ± 15.7%* | 41% ± 23.8%* |

*p-value < 0.05;
***p-value < 0.001

### Changes in Oil Profile in Knockout Lines Compared to Wild Type

Pea analysis for oil profiles was performed in knock out lines of pea plants whose lipoxygenases or desaturases were knocked out. The oil profiles of homogenates from pea seed flour of plants that had a knockout of LOX-2, FAD2B, FAD3C, a combination of FAD3C and FAD3D, or a combination of FAD2B, FAD3C, and FAD3D genes, were compared to homogenates of wild-type plants. SPME-GC analysis showed palmitic acid (16:0), steric acid (18:0), oleic acid (18:1), linoleic acid (18:2), and linolenic acid (18:3) to be major oil components in wild-type and knockout plants. The relative peak area was used to compare the intensity of the corresponding oil production in the knockout lines and wild-type plant. As shown in Table 9, there was a significant increase in oleic acid levels in the FAD3C knockout line compared to the wild type. Further, there was a significant reduction in linoleic acid levels in the FAD2B knockout line, as well as a significant reduction in linolenic acid levels in the FAD3C knockout line, FAD3C/FAD3D knockout line, and FAD2B/FAD3C/FAD3D knockout line compared to the wild type.

TABLE 9
Changes in Oil Profile in the Knockout Lines Compared to Wildtype

| | | % Palmitic | % Stearic | % Oleic | % Linoleic | % Linolenic |
|---|---|---|---|---|---|---|
| Plant ID | Genotype | (16:0) | (18:0) | (18:1) | (18:2) | (18:3) |
| Amigo | WT control | 13 ± 0.42 | 4 ± 0.57 | 29 ± 3.43 | 46 ± 3.40 | 9 ± 0.67 |
| Plant E | lox-2 | 12 ± 0.28* | 4 ± 0.12 | 27 ± 0.32 | 46 ± 0.31 | 11 ± 0.65* |
| Plant G | fad2b | 12 ± 0.30 | 5 ± 0.22 | 34 ± 1.11 | 38 ± 2.25* | 11 ± 1.10* |
| Plant H | fad3c | 12 ± 0.67 | 5 ± 0.35 | 38 ± 0.84* | 41 ± 1.14 | 4 ± 0.20*** |
| Plant I | fad3c/fad3d | 12 ± 0.13* | 5 ± 0.12 | 35 ± 0.40 | 44 ± 0.20 | 4 ± 0.25*** |
| Plant J | fad2b/fad3c/fad3d | 13 ± 0.12 | 4 ± 0.15 | 31 ± 1.24 | 48 ± 1.70 | 4 ± 0.23*** |

*p-value < 0.05;
***p-value < 0.001

### Example 5. Sensory Results of Plant Protein Isolates

Sensory tests are conducted as a blind taste test with pea slurries using 4 internal trained panelists. Briefly, a pea protein isolate is prepared using the methods know in the art (e.g., acid precipitation method as described in United States Patent Publication No.: US20190191735; incorporated by reference herein) and is used to prepare a patty like product. Textural characteristics of the patty like products from edited pea plants (e.g., LOX-2 edited, LOX-2 and LOX-3 edited, LOX-3 and FAD3C edited) are evaluated by a panel of trained sensory experts. Patties are formed and evaluated in uncooked and cooked state, and compared to patties obtained from WT pea plants. The samples are evaluated using a scorecard for a variety of attributes (e.g., surface color, browning, aroma, smell, surface texture, taste, oil content, hardness/firmness, chewiness, bite force, mouthfeel, degradation, fattiness, adhesiveness, elasticity, rubberiness, surface thickness, moldability, binding/integrity, grittiness, graininess, lumpiness, greasiness, moistness, sliminess) and quality factors (e.g., aroma, flavor, appearance, and texture, e.g.,). Reported are the consensus values of the perceived change relative to the wild type control. The five-point degree of difference (DOD) scale indicates the overall the perceived difference between sensory profiles of the test and control samples with while higher values representing bigger differences between samples (1=Match to Control, 2=Slightly Different, 3=Moderately Different, 4=Extremely Different, and 5=Reject). DOD differences less than 3 are considered natural variation and not significant. Additionally, 15 flavor attributes (overall aroma, overall flavor impact, beany yellow pea, pyrazine, cereal grain, green grassy green pea, nutty, cardboard, malty, salt, bitter, umami, astringent, chalky) are scored on a seven-point scale (−3 to 3) with positive values indicating favorable changes and negative values indicating unfavorable changes. Attribute differences greater than −1 and less than 1 are considered natural variation and not significant.

The resulting patty like product from edited pea plants (e.g., LOX-2 edited, LOX-2 and LOX-3 edited, LOX-3 and FAD3C edited) can have superior sensory characteristics (e.g., less odor, better flavor, less beany, less bitter, less astringent). In sum, pea protein isolates from edited pea plants (e.g., LOX-2 edited, LOX-2 and LOX-3 edited, LOX-3 and FAD3C edited) can demonstrate superior qualities with respect to sensory properties, in comparison to pea protein isolates from WT pea plants.

TABLE 10
Sequence Descriptions

| Sequence Identifier | Description |
|---|---|
| SEQ ID NO: 1 | Amino acids 11-25 of intact LOX-2 protein |
| SEQ ID NO: 2 | Amino acids 408-429 of intact LOX-2 protein |
| SEQ ID NO: 3 | Exon 4 of Pea LOX-2 gene |
| SEQ ID NO: 4 | Nucleic acid sequence encoding gRNA targeting region specific to exon 4 |
| | of Pea LOX-2 gene |
| SEQ ID NO: 5 | Exon 4 of Pea LOX-2 gene with 11 bp deletion, in Plant E |
| SEQ ID NO: 6 | Exon 4 of Pea LOX-2 gene with 8 bp deletion, in Plant B |
| SEQ ID NO: 7 | Derived amino acid sequence of the full-length Pea LOX-2 protein |
| SEQ ID NO: 8 | Derived amino acid sequence of the truncated Pea LOX-2 protein |
| SEQ ID NO: 9 | Derived amino acid sequence of the truncated Pea LOX-2 protein, in Plant B |
| SEQ ID NO: 10 | Nucleic acid sequence of the full-length Pea LOX-2 gene |
| SEQ ID NO: 11 | Nucleic acid sequence of Pea LOX-2 gene with 11 bp deletion, in Plant E |
| SEQ ID NO: 12 | Nucleic acid sequence of Pea LOX-2 gene with 8 bp deletion, in Plant B |
| SEQ ID NO: 13 | Forward primer 13062 (for LOX-2 gene) |
| SEQ ID NO: 14 | Reverse primer 13057 (for LOX-2 gene) |
| SEQ ID NO: 15 | gRNA binding sequence in exon 4 of Pea LOX-2 gene |
| SEQ ID NO: 16 | Amino acid sequence of Nuclease |
| SEQ ID NO: 17 | DNA fragment from WT pea plant |
| SEQ ID NO: 18 | DNA fragment from mutant pea plant with 8 bp deletion |
| SEQ ID NO: 19 | DNA fragment from mutant pea plant with 8 bp deletion |
| SEQ ID NO: 20 | DNA fragment from mutant pea plant with 12 bp deletion |
| SEQ ID NO: 21 | DNA fragment from mutant pea plant with 10 bp deletion |
| SEQ ID NO: 22 | exon 4 of Pea LOX-3 gene |
| SEQ ID NO: 23 | Nucleic acid sequence encoding gRNA targeting region specific to exon 4 |
| | of Pea LOX-3 gene |
| SEQ ID NO: 24 | Exon 4 of Pea LOX-3 gene from Plant F with 28 bp deletion |
| SEQ ID NO: 25 | Derived amino acid sequence of the full-length Pea LOX-3 protein |
| SEQ ID NO: 26 | Derived amino acid sequence of the truncated Pea LOX-3 protein |
| SEQ ID NO: 27 | Nucleic acid sequence of full-length Pea LOX-3 gene (introns and exons) |
| SEQ ID NO: 28 | Nucleic acid sequence of truncated Pea LOX-3 gene from Plant F with 28 |
| | bp deletion (introns and exons) |
| SEQ ID NO: 29 | Exon 1 of Pea FAD2B gene |
| SEQ ID NO: 30 | Nucleic acid sequence encoding gRNA targeting region specific to exon 1 |
| | of Pea FAD2B gene |
| SEQ ID NO: 31 | Exon 1 of Pea FAD2B gene from Plant G plant with 8 bp deletion |
| SEQ ID NO: 32 | Exon 1 of Pea FAD2B gene from Plant J with 2 bp deletion |
| SEQ ID NO: 33 | Derived amino acid sequence of the full-length Pea FAD2B protein |
| SEQ ID NO: 34 | Derived amino acid sequence of the truncated Pea FAD2B protein from −8 |
| | bp allele |
| SEQ ID NO: 35 | Derived amino acid sequence of the truncated Pea FAD2B protein from −2 |
| | bp allele |
| SEQ ID NO: 36 | Nucleic acid sequence of full-length Pea FAD2B gene (1 exon, no introns) |
| SEQ ID NO: 37 | Nucleic acid sequence of truncated Pea FAD2B gene from Plant G with 8 |
| | bp deletion (1 exon, no introns) |
| SEQ ID NO: 38 | Nucleic acid sequence of truncated Pea FAD2B gene from Plant J with 2 |
| | bp deletion (1 exon, no introns) |
| SEQ ID NO: 39 | Exon 2 of Pea FAD3C gene |
| SEQ ID NO: 40 | Nucleic acid sequence encoding gRNA targeting region specific to exon 2 |
| | of Pea FAD3C gene |
| SEQ ID NO: 41 | Exon 2 of Pea FAD3C gene from Plant H, Plant I, or Plant J with 8 bp |
| | deletion |
| SEQ ID NO: 42 | Exon 2 of Pea FAD3C gene from Plant I or Plant J plant with 49 bp deletion |
| SEQ ID NO: 43 | Derived amino acid sequence of the full-length Pea FAD3C protein |
| SEQ ID NO: 44 | Derived amino acid sequence of the truncated Pea FAD3C protein from −8 |
| | bp allele |
| SEQ ID NO: 45 | Derived amino acid sequence of the truncated Pea FAD3C protein from −49 |
| | bp allele |
| SEQ ID NO: 46 | Nucleic acid sequence of full-length Pea FAD3C gene (introns and exons) |
| SEQ ID NO: 47 | Nucleic acid sequence of truncated Pea FAD3C gene from Plant H, Plant I, |
| | or Plant J with 8 bp deletion (introns and exons) |
| SEQ ID NO: 48 | Nucleic acid sequence of truncated Pea FAD3C gene from Plant I or Plant J |
| | with 49 bp deletion (introns and exons) |
| SEQ ID NO: 49 | Exon 3 of Pea FAD3D gene |
| SEQ ID NO: 50 | Nucleic acid sequence encoding gRNA targeting region specific to exon 3 |
| | of Pea FAD3D gene |
| SEQ ID NO: 51 | Exon 3 of Pea FAD3D gene from Plant I with 5 bp deletion |
| SEQ ID NO: 52 | Exon 3 of Pea FAD3D gene from Plant I or Plant J with 107 bp deletion |
| SEQ ID NO: 53 | Derived amino acid sequence of the full-length Pea FAD3D protein |
| SEQ ID NO: 54 | Derived amino acid sequence of the truncated Pea FAD3D protein from −5 |
| | bp allele |
| SEQ ID NO: 55 | Derived amino acid sequence of the truncated Pea FAD3D protein from −107 |
| | bp allele |
| SEQ ID NO: 56 | Nucleic acid sequence of full-length Pea FAD3D gene (introns and exons) |
| SEQ ID NO: 57 | Nucleic acid sequence of truncated Pea FAD3D gene from Plant I with 5 bp |
| | deletion (introns and exons) |
| SEQ ID NO: 58 | Nucleic acid sequence of truncated Pea FAD3D gene from Plant I or Plant J |
| | with 107 bp deletion (introns and exons) |
| SEQ ID NO: 59 | Forward primer 13091 (for LOX-3 gene) |
| SEQ ID NO: 60 | Reverse primer 042 (for LOX-3 gene) |
| SEQ ID NO: 61 | Forward primer 005 (for FAD2B gene) |
| SEQ ID NO: 62 | Reverse primer 006 (for FAD2B gene) |
| SEQ ID NO: 63 | Forward primer 012 (for FAD3C gene) |
| SEQ ID NO: 64 | Reverse primer 020 (for FAD3C gene) |
| SEQ ID NO: 65 | Forward primer 021 (for FAD3D gene) |
| SEQ ID NO: 66 | Reverse primer 030 (for FAD3D gene) |

1. A plant or plant part comprising:
decreased liopoxygenase (LOX) activity, wherein the LOX activity in the said plant or plant part is decreased when compared to the LOX activity in a control plant or plant part expressing a wild-type LOX gene, and wherein said plant comprises one or more insertions, substitutions, or deletions in one or more genes selected from the group consisting of LOX-2 and LOX-3; and/or
decreased fatty acid desaturase (FAD) activity, wherein the FAD activity in the plant or plant part is decreased when compared to the FAD activity in a control plant or plant part, and wherein said plant comprises one or more insertions, substitutions, or deletions in one or more genes selected from the group consisting of FAD2 and FAD3.

2. (canceled)

3. The plant or plant part according to claim 1, wherein the amount of hexanal, hexanol, and/or linolenic acid is reduced relative to a control plant not comprising the one or more insertions, substitutions, or deletions in said one or more genes.

4.-9. (canceled)

10. The plant or plant part of claim 1, wherein the amount of oleic acid is increased relative to a control plant not comprising the one or more insertions, substitutions, or deletions in said one or more genes.

11.-15. (canceled)

16. The plant or plant part of claim 15, wherein said one or more insertions, substitutions, or deletions are located at least partially in the nucleotide region corresponding to
exon 4 of the LOX-2 gene,
exon 4 of the LOX-3 gene,
exon 1 of the FAD2B gene,
exon 2 of the FAD3C gene, and/or
exon 3 of the FAD3D gene.

17.-24. (canceled)

25. The plant or plant part according to claim 1, wherein said plant or plant part comprises
(i) SEQ ID NO: 5, or a deletion located in nucleotides 1521 through 1531 of SEQ ID NO: 10;
(ii) SEQ ID NO: 6, or a deletion located in nucleotides 1523 through 1530 of SEQ ID NO: 10;
(iii) SEQ ID NO: 24, or a deletion located in nucleotides 1129 through 1156 of SEQ ID NO: 27;
(iv) SEQ ID NO: 31, or a deletion located in nucleotides 59 through 66 of SEQ ID NO: 36;
(v) SEQ ID NO: 32, or a deletion located in nucleotides 60 through 61 of SEQ ID NO: 36;
(vi) SEQ ID NO: 41, or a deletion located in nucleotides 457 through 464 of SEQ ID NO: 46;
(vii) SEQ ID NO: 42, or a deletion located in nucleotides 416 through 464 of SEQ ID NO: 46;
(ix) SEQ ID NO: 51, or a deletion located in nucleotides 775 through 779 of SEQ ID NO: 56; and/or
(x) SEQ ID NO: 52, or a deletion located in nucleotides 745 through 851 of SEQ ID NO: 56.

26.-29. (canceled)

30. The plant or plant part according to claim 1, wherein said one or more insertions, substitutions, or deletions are located in a gene that encodes for a protein comprising:
a) an amino acid sequence having at least 90% sequence identity to the amino acid sequence set forth in SEQ ID NO: 7 and retains LOX-2 activity, or an amino acid sequence set forth in SEQ ID NO: 7;
b) an amino acid sequence having at least 90% sequence identity to the amino acid sequence set forth in SEQ ID NO: 25 and retains LOX-3 activity, or an amino acid sequence set forth in SEQ ID NO: 25;
c) an amino acid sequence having at least 90% sequence identity to the amino acid sequence set forth in SEQ ID NO: 33 and retains FAD2B activity, or an amino acid sequence set forth in SEQ ID NO: 33;
d) an amino acid sequence having at least 90% sequence identity to the amino acid sequence set forth in SEQ ID NO: 43 and retains FAD3C activity, or an amino acid sequence set forth in SEQ ID NO: 43; or
e) an amino acid sequence having at least 90% sequence identity to the amino acid sequence set forth in SEQ ID NO: 53 and retains FAD3D activity, or an amino acid sequence set forth in SEQ ID NO: 53.

31.-35. (canceled)

36. The plant or plant part according to claim 1, wherein the plant is selected from pea (Pisum sativum), bean (Phaseolus spp.), soybean (Glycine max), chickpea (Cicer arietinum), peanut (Arachis hypogaea), lentils (Lens culinaris, Lens esculenta), fava bean (Vicia faba), mung bean (Vigna radiata), lupins (Lupinus spp.), mesquite (Prosopis spp.), carob (Ceratonia siliqua), tamarind (Tamarindus indica), alfalfa (Medicago sativa), clover (Trifohum spp.), canola (Brassica napus), cotton (Gossypium sp.), camelina (Camelina sativa), sunflower (Hehanthus sp.), wheat (Triticum sp.), barley (Hordeum vulgare), maize (Zea mays), oats (Avena sativa), and hemp (Cannabis sativa).

37. A protein and/or oil composition isolated from the plant or plant part of claim 1, wherein said protein composition has decreased amount of hexanal, hexanol, and/or linolenic acid when compared to protein composition isolated from a control plant.

38. A method of decreasing the amount of hexanal, hexanol, and/or linolenic acid in a plant or plant part as compared to a control plant or plant part, said method comprising decreasing the activity of one or more genes in the plant selected from the group consisting of LOX-2, LOX-3, FAD2B, FAD3C, and FAD3D.

39. The method of claim 38, wherein decreasing the activity of one or more genes comprises introducing into said plant or plant part one or more insertions, substitutions, or deletions in in one or more genes selected from the group consisting of LOX-2, LOX-3, FAD2B, FAD3C, and FAD3D.

40.-46. (canceled)

47. The method according to claim 39, wherein
(i) said method comprises introducing a deletion comprising nucleotides 1521 through 1531 of SEQ ID NO: 10 into said plant or plant part, or said plant or plant part comprises SEQ ID NO: 5 after the deletion is introduced;
(ii) said method comprises introducing a deletion comprising nucleotides 1523 through 1530 of SEQ ID NO: 10 into said plant or plant part, or said plant or plant part comprises SEQ ID NO: 6 after the deletion is introduced;
(iii) said method comprises introducing a deletion comprising nucleotides 1129 through 1156 of SEQ ID NO: 27 into said plant or plant part, or said plant or plant part comprises SEQ ID NO: 24 after the deletion is introduced;
(iv) said method comprises introducing a deletion comprising nucleotides 59 through 66 of SEQ ID NO: 36 into said plant or plant part, or said plant or plant part comprises SEQ ID NO: 31 after the deletion is introduced;
(v) said method comprises introducing a deletion comprising nucleotides 60 through 61 of SEQ ID NO: 36 into said plant or plant part, or said plant or plant part comprises SEQ ID NO: 32 after the deletion is introduced;
(vi) said method comprises introducing a deletion comprising nucleotides 457 through 464 of SEQ ID NO: 46 into said plant or plant part, or said plant or plant part comprises SEQ ID NO: 41 after the deletion is introduced;
(viii) said method comprises introducing a deletion comprising nucleotides 416 through 464 of SEQ ID NO: 46 into said plant or plant part, or said plant or plant part comprises SEQ ID NO: 42 after the deletion is introduced;
(ix) said method comprises introducing a deletion comprising nucleotides 775 through 779 of SEQ ID NO: 56 into said plant or plant part, or said plant or plant part comprises SEQ ID NO: 51 after the deletion is introduced; and/or
(x) said method comprises introducing a deletion comprising nucleotides 745 through 851 of SEQ ID NO: 56 into said plant or plant part, or said plant or plant part comprises SEQ ID NO: 52 after the deletion is introduced.

48. (canceled)

49. The method of claim 38, comprising introducing into said plant a nucleic acid construct encoding at least one site-directed nuclease that is specific for a target site in a genome of the plant, wherein, upon expression, the at least one nuclease cleaves the plant genome at the target site, wherein one or more insertions, substitutions, or deletions are introduced at the target site and the activity of one or more genes selected from the group consisting of LOX-2, LOX-3, FAD2B, FAD3C, and FAD3D is decreased.

50.-54. (canceled)

55. The method of claim 49, further comprising introducing into said plant or plant part at least one guide RNA (gRNA) operatively arranged with the nuclease, wherein the gRNA binds the target site and guides the nuclease to the target site, wherein the target site is located in the one or more genes selected from the group consisting of LOX-2, LOX-3, FAD2B, FAD3C, and FAD3D.

56.-70. (canceled)

71. A protein and/or oil composition isolated from a plant or plant part obtained by the method of claim 38, wherein said protein and/or oil composition comprises: one or more nucleic acid molecules each comprising a nucleic acid sequence of a mutated LOX-2, LOX-3, FAD2B, FAD3C, or FAD3D gene or fragment thereof; and/or decreased hexanal, hexanol, and/or linolenic acid amounts as compared to a protein and/or oil composition isolated from a control plant.

72. The protein and/or oil composition of claim 71, wherein said protein and/or oil composition is isolated from a pea plant.

73. Oil produced from the plant or plant part of claim 1, wherein the oil comprises:
high oleic acid content;
low linoleic acid content;
low linolenic acid content;
high oleic acid and low linoleic acid content;
high oleic acid and low linolenic acid content;
low linoleic acid and low linolenic acid content; or
high oleic acid, low linoleic acid, and low linolenic acid content, relative to oil produced from a control plant or plant part.

74.-77. (canceled)

78. Plant oil comprising a linolenic acid content of about 4% to about 10%.

79. The plant oil of claim 78, comprising an oleic acid content of about 30% to about 40%, and a linoleic plus linolenic acid content of about 45% to 55%.

80. The oil of claim 78, comprising one or more nucleic acid molecules each comprising a nucleic acid sequence of a mutated LOX-2, LOX-3, FAD2 or FAD3 gene or fragment thereof.

81. The oil of claim 80, wherein said one or more nucleic acid molecules each comprise a nucleic acid sequence of:
a mutated LOX-2 gene or a fragment thereof comprising a deletion of nucleotides 1521 through 1531 of SEQ ID NO: 10;
(ii) a mutated LOX-2 gene or a fragment thereof comprising a deletion of nucleotides 1523 through 1530 of SEQ ID NO: 10;
(iii) a mutated LOX-3 gene or a fragment thereof comprising a deletion of nucleotides 1129 through 1156 of SEQ ID NO: 27;
(iv) a mutated FAD2B gene or a fragment thereof comprising a deletion of nucleotides 59 through 66 of SEQ ID NO: 36;
(v) a mutated FAD2B gene or a fragment thereof comprising a deletion of nucleotides 60 through 61 of SEQ ID NO: 36;
(vi) a mutated FAD3C gene or a fragment thereof comprising a deletion of nucleotides 457 through 464 of SEQ ID NO: 46;
(vii) a mutated FAD3C gene or a fragment thereof comprising a deletion of nucleotides 416 through 464 of SEQ ID NO: 46;
(viii) a mutated FAD3D gene or a fragment thereof comprising a deletion of nucleotides 775 through 779 of SEQ ID NO: 56; or
(ix) a mutated FAD3D gene or a fragment thereof comprising a deletion of nucleotides 745 through 851 of SEQ ID NO: 56.

82. (canceled)
